(19) United States
(12) Patent Application Publication
(10) Pub. No.: US 2021/0395374 A1
(43) Pub. Date: Dec. 23, 2021

(54) Bispecific CD123 x CD3 Diabodies for the Treatment of Hematologic Malignancies

(71) Applicants: MacroGenics, Inc., Rockville, MD (US); NanoString Technologies, Inc., Seattle, WA (US); Nottingham Trent University, Nottingham (GB)

(72) Inventors: Jan Kenneth Davidson, Rockville, MD (US); Sara Church, Everett, WA (US); Sergio Rutella, Nottingham (GB)

(73) Assignees: MacroGenics, Inc., Rockville, MD (US); NanoString Technologies, Inc., Seattle, WA (US); Nottingham Trent University, Nottingham (GB)

(21) Appl. No.: 17/290,061

(22) PCT Filed: Oct. 29, 2019
(86) PCT No.: PCT/US2019/058616

§ 371 Date: Apr. 29, 2021

Related U.S. Application Data
(60) Provisional application No. 62/752,659, filed on Oct. 30, 2018.
(60) Provisional application No. 62/769,078, filed on Nov. 19, 2018.
(60) Provisional application No. 62/878,368, filed on Jul. 25, 2019.

(51) Int. Cl. C07K 16/28; A61P 35/02

(52) U.S. Cl. CPC C07K 16/2866; C07K 16/2809; A61K 39/00; C07K 2317/31; A61P 35/02

(57) ABSTRACT

The present invention is directed to a method of treating a hematologic malignancy such as acute myeloid leukemia (AML) or myelodysplastic syndrome (MDS), including hematologic malignancies that are refractive to chemotherapeutic and/or hypomethylating agents. The method concerns administering a CD123×CDS bispecific binding molecule to a patient in an amount effective to stimulate the killing of cells of said hematologic malignancy in said patient. The present invention is additionally directed to the embodiment of such method in which a cellular sample from the patient evidences an expression of one or more target genes that is increased relative to a baseline level of expression of such genes, for example, a baseline level of expression of such genes in a reference population of individuals who are suffering from the hematologic malignancy, or with respect to the level of expression of a reference gene.

### CROSS-REFERENCE TO RELATED APPLICATIONS

This application claims priority to U.S. Patent Applications Ser. No. 62/878,368 (filed on Jul. 25, 2019; pending), 62/769,078 (filed on Nov. 19, 2018; pending) and 62/752,659 (filed on Oct. 30, 2018; pending), each of which applications is herein incorporated by reference in its entirety.

### REFERENCE TO SEQUENCE LISTING

This application includes one or more Sequence Listings pursuant to 37 C.F.R. 1.821 et seq., which are disclosed in computer-readable media (file name: 1301_0161PCT_ST25.txt, created on Sep. 26, 2019, and having a size of 31,244 bytes), which file is herein incorporated by reference in its entirety.

### FIELD OF THE INVENTION

The present invention is directed to a method of treating a hematologic malignancy such as acute myeloid leukemia (AML) or myelodysplastic syndrome (MDS), including hematologic malignancies that are refractive to chemotherapeutic and/or hypomethylating agents. The method concerns administering a CD123×CD3 bispecific binding molecule to a patient in an amount effective to stimulate the killing of cells of said hematologic malignancy in said patient. The present invention is additionally directed to the embodiment of such method in which a cellular sample from the patient evidences an expression of one or more target genes that is increased relative to a baseline level of expression of such genes, for example, a baseline level of expression of such genes in a reference population of individuals who are suffering from the hematologic malignancy, or with respect to the level of expression of a reference gene.

### BACKGROUND OF THE INVENTION

I. CD123

CD123 (interleukin 3 receptor alpha, IL-3Ra) is a 40 kDa molecule and is part of the interleukin 3 receptor complex (Stomski, F. C. et al. (1996) “Human Interleukin-3 (IL-3) Induces Disulfide-Linked IL-3 Receptor Alpha-And Beta-Chain Heterodimerization, Which Is Required For Receptor Activation But Not High-Affinity Binding,” Mol. Cell. Biol. 16(6):3035-3046). Interleukin 3 (IL-3) drives early differentiation of multipotent stem cells into cells of the erythroid, myeloid and lymphoid progenitors. CD123 is expressed on CD34+ committed progenitors (Taussig, D. C. et al. (2005) “Hematopoietic Stem Cells Express Multiple Myeloid Markers: Implications For The Origin And Targeted Therapy Of Acute Myeloid Leukemia,” Blood 106:4086-4092), but not by CD34+/CD38− normal hematopoietic stem cells. CD123 is expressed by basophils, mast cells, plasmacytoid dendritic cells, some expression by monocytes, macrophages and eosinophils, and low or no expression by neutrophils and megakaryocytes. Some non-hematopoietic tissues (placenta, Leydig cells of the testis, certain brain cell elements and some endothelial cells) express CD123; however, expression is mostly cytoplasmic.

CD123 is reported to be expressed by leukemic blasts and leukemia stem cells (LSC) (Jordan, C. T. et al. (2000) “The Interleukin-3 Receptor Alpha Chain Is A Unique Marker For Human Acute Myelogenous Leukemia Stem Cells,” Leukemia 14:1777-1784; Jin, W. et al. (2009) “Regulation Of Th17 Cell Differentiation And EAE Induction By MAP3K NIK,” Blood 113:6603-6610). In human normal precursor populations, CD123 is expressed by a subset of hematopoietic progenitor cells (HPC) but not by normal hematopoietic stem cells (HSC). CD123 is also expressed by plasmacytoid dendritic cells (pDC) and basophils, and, to a lesser extent, monocytes and eosinophils (Lopez, A. F. et al. (1989) “Reciprocal Inhibition Of Binding Between Interleukin 3 And Granulocyte-Macrophage Colony-Stimulating Factor To Human Eosinophils,” Proc. Natl. Acad. Sci. (U.S.A.) 86:7022-7026; Sun, Q. et al. (1996) “Monoclonal Antibody 7G3 Recognizes The N-Terminal Domain Of The Human Interleukin-3 (IL-3) Receptor Alpha Chain And Functions As A Specific IL-3 Receptor Antagonist,” Blood 87:83-92; Muñoz, L. et al. (2001) “Interleukin-3 Receptor Alpha Chain (CD123) Is Widely Expressed In Hematologic Malignancies,” Haematologica 86(12):1261-1269; Masten, B. J. et al. (2006) “Characterization Of Myeloid And Plasmacytoid Dendritic Cells In Human Lung,” J. Immunol. 177:7784-7793; Korpelainen, E. I. et al. (1995) “Interferon-Gamma Upregulates Interleukin-3 (IL-3) Receptor Expression In Human Endothelial Cells And Synergizes With IL-3 In Stimulating Major Histocompatibility Complex Class II Expression And Cytokine Production,” Blood 86:176-182).

CD123 has been reported to be overexpressed on malignant cells in a wide range of hematologic malignancies including acute myeloid leukemia (AML) and myelodysplastic syndrome (MDS) (Muñoz, L. et al. (2001) “Interleukin-3 Receptor Alpha Chain (CD123) Is Widely Expressed In Hematologic Malignancies,” Haematologica 86(12):1261-1269). Overexpression of CD123 is associated with poorer prognosis in AML (Tettamanti, M. S. et al. (2013) “Targeting Of Acute Myeloid Leukaemia By Cytokine-Induced Killer Cells Redirected With A Novel CD123-Specific Chimeric Antigen Receptor,” Br. J. Haematol. 161:389-401).

II. CD3

CD3 is a T cell co-receptor composed of four distinct chains (Wucherpfennig, K. W. et al. (2010) “Structural Biology Of The T-Cell Receptor: Insights Into Receptor Assembly, Ligand Recognition, And Initiation Of Signaling,” Cold Spring Harb. Perspect. Biol. 2(4):a005140; pages 1-14). In mammals, the complex contains a CD3γ chain, a CD3δ chain, and two CD3ε chains. These chains associate with a molecule known as the T cell receptor (TCR) in order to generate an activation signal in T lymphocytes. In the absence of CD3, TCRs do not assemble properly and are degraded (Thomas, S. et al. (2010) “Molecular Immunology Lessons From Therapeutic T-Cell Receptor Gene Transfer,” Immunology 129(2):170-177). CD3 is found bound to the membranes of all mature T cells, and in virtually no other cell type (see, Janeway, C. A. et al. (2005) In: IMMUNOBIOLOGY: THE IMMUNE SYSTEM IN HEALTH AND DISEASE,” 6th Ed., Garland Science Publishing, NY, pp. 214-216; Sun, Z. J. et al. (2001) “Mechanisms Contributing To T Cell Receptor Signaling And Assembly Revealed By The Solution Structure Of An Ectodomain Fragment Of The CD3ε:γ Heterodimer,” Cell 105(7):913-923; Kuhns, M. S. et al. (2006) “Deconstructing The Form And Function Of The TCR/CD3 Complex,” Immunity. 2006 February; 24(2):133-139).

III. AML and MDS

Acute myeloid leukemia (AML) and myelodysplastic syndrome (MDS) are thought to arise in, and be perpetuated by, a small population of leukemic stem cells (LSCs), which are generally dormant (i.e., not rapidly dividing cells) and therefore resist cell death (apoptosis) and conventional chemotherapeutic agents. LSCs are characterized by high levels of CD123 expression, which is not present in the corresponding normal hematopoietic stem cell population in normal human bone marrow (Jin, W. et al. (2009) “Regulation Of Th17 Cell Differentiation And EAE Induction By MAP3K NIK,” Blood 113:6603-6610; Jordan, C. T. et al. (2000) “The Interleukin-3 Receptor Alpha Chain Is A Unique Marker For Human Acute Myelogenous Leukemia Stem Cells,” Leukemia 14:1777-1784). CD123 is expressed in 45%-95% of AML, 85% of Hairy cell leukemia (HCL), and 40% of acute B lymphoblastic leukemia (B-ALL). CD123 expression is also associated with multiple other malignancies/pre-malignancies: chronic myeloid leukemia (CML) progenitor cells (including blast crisis CML); Hodgkin's Reed Sternberg (RS) cells; transformed non-Hodgkin's lymphoma (NHL); some chronic lymphocytic leukemia (CLL) (CD11c+); a subset of acute T lymphoblastic leukemia (T-ALL) (16%, most immature, mostly adult), plasmacytoid dendritic cell (pDC) DC2 malignancies and CD34+/CD38− myelodysplastic syndrome (MDS) marrow cell malignancies.

AML is a clonal disease characterized by the proliferation and accumulation of transformed myeloid progenitor cells in the bone marrow, which ultimately leads to hematopoietic failure. The incidence of AML increases with age, and older patients typically have worse treatment outcomes than younger patients (Robak, T. et al. (2009) “Current And Emerging Therapies For Acute Myeloid Leukemia,” Clin. Ther. 2:2349-2370). Unfortunately, at present, most adults with AML die from their disease.

Treatment for AML initially focuses in the induction of remission (induction therapy). Once remission is achieved, treatment shifts to focus on securing such remission (post-remission or consolidation therapy) and, in some instances, maintenance therapy. The standard remission induction paradigm for AML is chemotherapy with an anthracycline/cytarabine combination, followed by either consolidation chemotherapy (usually with higher doses of the same drugs as were used during the induction period) or human stem cell transplantation, depending on the patient's ability to tolerate intensive treatment and the likelihood of cure with chemotherapy alone (see, e.g., Roboz, G. J. (2012) “Current Treatment Of Acute Myeloid Leukemia,” Curr. Opin. Oncol. 24:711-719).

Agents frequently used in induction therapy include cytarabine and the anthracyclines. Cytarabine (also known as AraC) kills cancer cells (and other rapidly dividing normal cells) by interfering with DNA synthesis. Side effects associated with AraC treatment include decreased resistance to infection, a result of decreased white blood cell production; bleeding, as a result of decreased platelet production; and anemia, due to a potential reduction in red blood cells. Other side effects include nausea and vomiting. Anthracyclines (e.g., daunorubicin, doxorubicin, and idarubicin) have several modes of action including inhibition of DNA and RNA synthesis, disruption of higher order structures of DNA, and production of cell damaging free oxygen radicals. The most consequential adverse effect of anthracyclines is cardiotoxicity, which considerably limits administered life-time dose and to some extent their usefulness.

Stem cell transplantation has been established as the most effective form of anti-leukemic therapy in patients with AML in first or subsequent remission (Roboz, G. J. (2012) “Current Treatment Of Acute Myeloid Leukemia,” Curr. Opin. Oncol. 24:711-719). However, unfortunately, despite substantial progress in the treatment of newly diagnosed AML, 20% to 40% of patients do not achieve remission with the standard induction chemotherapy, and 50% to 70% of patients entering a first complete remission are expected to relapse within 3 years. The optimum strategy at the time of relapse, or for patients with the resistant disease, remains uncertain (see, Tasian, S. K. (2018 “Acute Myeloid Leukemia Chimeric Antigen Receptor T-Cell Immunotherapy: How Far Up The Road Have We Traveled?,” Ther. Adv. Hematol. 9(6):135-148; Przespolewski, A. et al. (2018) “Advances In Immunotherapy For Acute Myeloid Leukemia” Future Oncol. 14(10):963-978; Shimabukuro-Vornhagen, A. et al. (2018) “Cytokine Release Syndrome,” J. Immunother. Cancer. 6(1):56 pp. 1-14; Milone, M. C. et al. (2018) “The Pharmacology of T Cell Therapies,” Mol. Ther. Methods Clin. Dev. 8:210-221; Dhodapkar, M. V. et al. (2017) “Hematologic Malignancies: Plasma Cell Disorders,” Am. Soc. Clin. Oncol. Educ. Book. 37:561-568; Kroschinsky, F. et al. (2017) “New Drugs, New Toxicities: Severe Side Effects Of Modern Targeted And Immunotherapy Of Cancer And Their Management,” Crit. Care 14; 21(1):89). Thus, novel therapeutic strategies are needed.

IV. Bispecific Molecules

The provision of non-monospecific molecules (e.g., bispecific antibodies, bispecific diabodies, BiTE® antibodies, etc.) provides a significant advantage over monospecific molecules such as natural antibodies: the capacity to co-ligate and co-localize cells that express different epitopes. Bispecific molecules thus have wide-ranging applications including therapy and immunodiagnosis. Bispecificity allows for great flexibility in the design and engineering of the diabody in various applications, providing enhanced avidity to multimeric antigens, the cross-linking of differing antigens, and directed targeting to specific cell types relying on the presence of both target antigens. Of particular importance is the co-ligating of differing cells, for example, the cross-linking of effector cells, such as cytotoxic T cells, to tumor cells (Staerz et al. (1985) “Hybrid Antibodies Can Target Sites For Attack By T Cells,” Nature 314:628-631, and Holliger et al. (1996) “Specific Killing Of Lymphoma Cells By Cytotoxic T-Cells Mediated By A Bispecific Diabody,” Protein Eng. 9:299-305).

In order to provide molecules having greater capability than natural antibodies, a wide variety of recombinant bispecific antibody formats have been developed (see, e.g., PCT Publication Nos. WO 2008/003116, WO 2009/132876, WO 2008/003103, WO 2007/146968, WO 2009/018386, WO 2012/009544, WO 2013/070565), most of which use linker peptides either to fuse a further binding protein (e.g., an scFv, VL, VH, etc.) to, or within, the antibody core (IgA, IgD, IgE, IgG or IgM), or to fuse multiple antibody binding portions (e.g., two Fab fragments or scFvs) to one another. Alternative formats use linker peptides to fuse a binding protein (e.g., an scFv, VL, VH, etc.) to a dimerization domain, such as the CH2-CH3 Domain, or to alternative polypeptides (WO 2005/070966, WO 2006/107786 WO 2006/107617, WO 2007/046893) and other formats in which the CL and CH1 Domains are switched from their respective natural positions and/or the VL and VH Domains have been diversified (WO 2008/027236; WO 2010/108127) to allow them to bind to more than one antigen.

The art has additionally noted the capability to produce diabodies that are capable of binding two or more different epitope species (see, e.g., Holliger et al. (1993) “‘Diabodies’: Small Bivalent And Bispecific Antibody Fragments,” Proc. Natl. Acad. Sci. (U.S.A.) 90:6444-6448. Stable, covalently bonded heterodimeric non-monospecific diabodies have been described (see, e.g., WO 2006/113665; WO/2008/157379; WO 2010/080538; WO 2012/018687; WO/2012/162068; Johnson, S. et al. (2010) “Effector Cell Recruitment With Novel Fv-Based Dual-Affinity Re-Targeting Protein Leads To Potent Tumor Cytolysis And In Vivo B-Cell Depletion,” J. Molec. Biol. 399(3):436-449; Veri, M. C. et al. (2010) “Therapeutic Control Of B Cell Activation Via Recruitment Of Fcgamma Receptor IIb (CD32B) Inhibitory Function With A Novel Bispecific Antibody Scaffold,” Arthritis Rheum. 62(7): 1933-1943; Moore, P. A. et al. (2011) “Application Of Dual Affinity Retargeting Molecules To Achieve Optimal Redirected T-Cell Killing Of B-Cell Lymphoma,” Blood 117(17):4542-4551). Such diabodies incorporate one or more cysteine residues into each of the employed polypeptide species. For example, the addition of a cysteine residue to the C-terminus of such constructs has been shown to allow disulfide bonding between the polypeptide chains, stabilizing the resulting heterodimer without interfering with the binding characteristics of the bivalent molecule. In addition, trivalent molecules comprising a diabody-like domain have been described (see, e.g., WO 2015/184203; and WO 2015/184207). Diabody epitope binding domains may also be directed to a surface determinant of any immune effector cell such as CD3, CD16, CD32, or CD64, which are expressed on T lymphocytes, natural killer (NK) cells or other mononuclear cells. In many studies, diabody binding to effector cell determinants, e.g., Fcγ receptors (FcγR), was also found to activate the effector cell (Holliger et al. (1996) “Specific Killing Of Lymphoma Cells By Cytotoxic T-Cells Mediated By A Bispecific Diabody,” Protein Eng. 9:299-305; Holliger et al. (1999) “Carcinoembryonic Antigen (CEA)-Specific T-cell Activation In Colon Carcinoma Induced By Anti-CD3×Anti-CEA Bispecific Diabodies And B7×Anti-CEA Bispecific Fusion Proteins,” Cancer Res. 59:2909-2916; WO 2006/113665; WO 2008/157379; WO 2010/080538; WO 2012/018687; WO 2012/162068). Normally, effector cell activation is triggered by the binding of an antigen-bound antibody to an effector cell via Fc-FcγR interaction; thus, in this regard, diabody molecules may exhibit Ig-like functionality independent of whether they comprise an Fc Domain (e.g., as assayed in any effector function assay known in the art or exemplified herein (e.g., ADCC assay)). By cross-linking tumor and effector cells, the diabody not only brings the effector cell within the proximity of the tumor cell, but leads to effective tumor killing (see e.g., Cao et al. (2003) “Bispecific Antibody Conjugates In Therapeutics,” Adv. Drug. Deliv. Rev. 55:171-197).

Several bispecific molecules targeting CD123 and CD3 capable of mediating T cell redirected cell killing of CD123-expressing malignant cells are in development (see, e.g., Vey, N., et al. (2017) “Interim Results From A Phase 1 First-In-Human Study Of Flotetuzumab, a CD123×CD3 Bispecific DART Molecule In AML/MDS,” Annals of Oncology, 28(S5)5, mdx373.001; Godwin, C. D., et al. (2017) “Bispecific Anti-CD123×Anti-CD3 Adaptir™ Molecules APVO436 and APVO437 Have Broad Activity Against Primary Human AML Cells In Vitro” Blood. 130(S1): 2639; Forslund, A., et al. (2016) “Ex Vivo Activity Profile of the CD123×CD3 Duobody® Antibody JNJ-63709178 Against Primary Acute Myeloid Leukemia Bone Marrow Samples” Blood 128(22):2875.). However, efforts to employ bispecific binding molecules that are capable of targeting a T cell to the location of a hematologic malignancy have not been fully successful. Hence, an unmet need remains to develop new strategies for the treatment of hematologic malignancies with CD123×CD3 bispecific binding molecules. The present invention directly addresses this need and others, as described below.

### SUMMARY OF THE INVENTION

The present invention is directed to a method of treating a hematologic malignancy such as acute myeloid leukemia (AML) or myelodysplastic syndrome (MDS), including hematologic malignancies that are refractive to chemotherapeutic and/or hypomethylating agents. The method concerns administering a CD123×CD3 bispecific binding molecule to a patient in an amount effective to stimulate the killing of cells of the hematologic malignancy in the patient. The present invention is additionally directed to the embodiment of such method in which a cellular sample from the patient evidences an expression of one or more target genes that is increased relative to a baseline level of expression of such genes, for example, a baseline level of expression of such genes in a reference population of individuals who are suffering from the hematologic malignancy, or with respect to the level of expression of a reference gene.

In detail, the invention provides a method of treating a chemo-refractory hematologic malignancy in a patient, wherein the method comprises administering to the patient a treatment dosage of a CD123×CD3 bispecific molecule, the dosage being effective to stimulate the killing of cells of the hematologic malignancy in the patient and thereby treat such malignancy.

The invention further provides the embodiment of such methods that additionally comprises evaluating the expression of one or more target and/or reference genes in a cellular sample from the patient, prior to and/or subsequent to the administration of the CD123×CD3 bispecific molecule. The invention further provides, the embodiment of such methods wherein the method comprises evaluating the expression of such one or more target and/or such one or more reference genes prior to the administration of the CD123×CD3 bispecific molecule. The invention also provides the embodiment of such methods wherein the method comprises evaluating the expression of such one or more target and/or such one or more reference genes subsequent to the administration of the CD123×CD3 bispecific molecule.

The invention further provides a method of determining whether a patient would be a suitable responder to the use of a CD123×CD3 bispecific molecule to treat a hematologic malignancy, wherein the method comprises:
(a) evaluating the expression of one or more target genes in a cellular sample from the patient prior to the administration of the CD123×CD3 bispecific molecule, relative to the expression of one or more target and/or reference genes; and
(b) identifying the patient as a suitable responder for treatment with a CD123×CD3 bispecific molecule if the expression of the one or more target genes is found to be increased relative to the expression of the one or more target and/or reference genes.

The invention further provides the embodiment of such methods wherein the method evaluates: (i) the expression of one or more target gene; and (ii) one or more reference gene whose expression is not characteristically associated with the hematologic malignancy.

The invention further provides the embodiment of such methods that comprises evaluating the expression of the one or more target genes relative to the baseline expression of the one or more reference genes of the patient.

The invention further provides the embodiment of such methods that comprises evaluating the expression of the one or more target genes of a patient relative to the expression of the one or more target genes of an individual who is suffering from the hematologic malignancy or of a population of such individuals. The invention further provides the embodiment of such methods wherein the expression of the one or more target genes of such patient is greater than the first quartile (i.e., greater than the bottom 25%), greater than the second quartile (i.e., greater than the bottom 50%), or greater than the third quartile (i.e., greater than the bottom 75%) of the expression levels of such target gene(s) of such individual or of such population of individuals who are suffering from the hematologic malignancy.

The invention further provides the embodiment of such methods that comprises evaluating the expression of the one or more target genes of a patient relative to the expression of the one or more target genes of an individual who had previously been unsuccessfully treated for a hematologic malignancy using the methods and compositions of the present invention (e.g., an individual who did not successfully respond to a treatment for a hematologic malignancy using a CD123×CD3 bispecific molecule), or a population of such individuals. The invention further provides the embodiment of such methods wherein the expression of the one or more target genes of such patient is greater than the first quartile (i.e., greater than the bottom 25%), greater than the second quartile (i.e., greater than the bottom 50%), or greater than the third quartile (i.e., greater than the bottom 75%) of the expression levels of such target gene(s) of such individual or of such population of unsuccessfully treated individuals. The invention further provides the embodiment of such methods wherein the expression of the one or more target genes of such patient has a log₂-fold change of at least about 0.4, at least about 0.5, at least about 0.6, or higher relative to the expression levels of such target gene(s) of such individual or such population of unsuccessfully treated individuals.

The invention further provides the embodiment of such methods that comprises evaluating the expression of the one or more target genes of a patient relative to the expression of the one or more target genes of an individual who had previously been successfully treated for a hematologic malignancy using the methods and compositions of the present invention (e.g., an individual who successfully responded to a treatment for a hematologic malignancy using a CD123×CD3 bispecific molecule) or a population of such individuals. The invention further provides the embodiment of such methods wherein the expression of the one or more target genes of such patient is within the first quartile (i.e., within the bottom 25%) of the expression levels of such target gene(s), within the second quartile (i.e., between the bottom 25% and 50%), or within the third quartile (i.e., between the bottom 50% and 75%) of the expression levels of such target gene(s) of such individual or such population of successfully treated individuals.

The invention further provides the embodiment of such methods wherein the relative expression level of the one or more target genes in the population is established by averaging the gene expression level in cellular samples obtained from the population of individuals.

The invention further provides the embodiment of such methods wherein such patient exhibits an expression level of at least one of such target genes:
(a) that is greater than the first quartile of the expression levels of such target gene in a population of individuals who are suffering from the hematologic malignancy; or
(b) that is greater than the first quartile of the expression levels of such target gene in a population of individuals who did not successfully respond to a treatment for the hematologic malignancy that used a CD123×CD3 bispecific molecule; or
(c) that has a log₂-fold change of at least about 0.4 relative to the expression levels of such target gene in a population of individuals who did not successfully respond to a treatment for the hematologic malignancy that used a CD123×CD3 bispecific molecule; or
(d) that is within at least the first quartile of the expression levels of such target gene in a population of individuals who successfully responded to a treatment for a hematologic malignancy that used a CD123×CD3 bispecific molecule.

The invention further provides the embodiment of such methods wherein such patient exhibits an expression level of at least one of such target genes:
(a) that is greater than the second quartile of the expression levels of such target gene in a population of individuals who are suffering from the hematologic malignancy; or
(b) that is greater than the second quartile of the expression levels of such target gene in a population of individuals who did not successfully respond to a treatment for the hematologic malignancy that used a CD123×CD3 bispecific molecule; or
(c) that has a log₂-fold change of at least about 0.4 relative to the expression levels of such target gene in a population of individuals who did not successfully respond to a treatment for the hematologic malignancy that used a CD123×CD3 bispecific molecule; or
(d) that is within at least the second quartile of the expression levels of such target gene in a population of individuals who successfully responded to a treatment for the hematologic malignancy that used a CD123×CD3 bispecific molecule.

The invention further provides the embodiment of such methods wherein such patient exhibits an expression level of at least one of such target genes:
(a) that is greater than the third quartile of the expression levels of such target gene in a population of individuals who are suffering from said hematologic malignancy; or
(b) that is greater than the third quartile of the expression levels of such target gene in a population of individuals who did not successfully respond to a treatment for said hematologic malignancy that used a CD123×CD3 bispecific molecule; or
(c) that has a log₂-fold change of at least about 0.6 relative to the expression levels of such target gene in a population of individuals who did not successfully respond to a treatment for said hematologic malignancy that used a CD123×CD3 bispecific molecule.

The invention further provides a method of treating a hematologic malignancy, wherein the method comprises:
(a) employing the method of any one of the above embodiments to determine whether a patient would be a suitable responder to the use of a CD123×CD3 bispecific molecule to treat the hematologic malignancy;
(b) administering a treatment dosage of the CD123×CD3 bispecific molecule to the patient if the patient is determined to be a suitable responder to such treatment;
   wherein the administration of the CD123×CD3 bispecific molecule stimulates the killing of cells of the hematologic malignancy in the patient.

The invention further provides the embodiment of such methods that additionally comprises evaluating the expression of such one or more target genes in a cellular sample obtained from the patient one or more times after the initiation of the treatment.

The invention further provides the embodiment of such methods wherein the cellular sample is a bone marrow or a blood sample. Particularly, the embodiment of such methods wherein the cellular sample is a bone marrow sample.

The invention further provides the embodiment of such methods that further comprises detecting the expression level of one or more target genes in a sample of the patient's bone marrow. The invention further provides the embodiment of such methods that further comprises detecting the expression level of one or more reference genes.

The invention further provides the embodiment of such methods that comprise detecting the expression level of such one or more target genes and/or such one or more reference genes in a sample of the patient's bone marrow, particularly prior to administration of a CD123×CD3 bispecific molecule.

The invention further provides the embodiment of such methods wherein the evaluation of expression or the determination of whether the patient would be a suitable responder to the use of a CD123×CD3 bispecific molecule to treat a hematologic malignancy is performed by:
(a) determining the gene expression levels for each target gene in one or more cellular sample(s) using a gene expression platform; and
(b) comparing the target gene expression levels to the expression levels of one or more reference genes.

The invention further provides the embodiment of such methods wherein the evaluation of expression or the determination of whether the patient would be a suitable responder to the use of a CD123×CD3 bispecific molecule to treat a hematologic malignancy is performed by:
(a) measuring the raw RNA levels for each target gene in one or more cellular sample(s) in a gene expression platform;
   wherein the gene expression platform comprises a reference gene set of housekeeping genes; and
(b) assigning a relative expression value, for each of the measured raw RNA levels for the target genes using the measured RNA levels of the internal reference genes.

The invention further provides the embodiment of such methods wherein the one or more target genes comprise:
(a) one or more of: CXCL9, CXCL10, CXCL11, and STAT1; and or
(b) one or more of: CCL5, CD27, CD274, CD276, CD8A, CMKLR1, CXCL9, CXCR6, HLA-DQA1, HLA-DRB1, HLA-E, IDO1, LAG3, NKG7, PDCD1LG2, PSMB10, STAT1, and TIGIT; and/or
(c) one or more of: AREG, CSF3, CXCL1, CXCL2, CXCL3, CCL20, FOSL1, IER3 (NM_003897.4), IL6 and PTGS2; and/or
(d) one or more of: CCL2, CCL3/L1, CCL4, CCL7 and CCL8; and/or
(e) one or more of: MAGEA3/A6, MAGEA1, MAGEA12, MAGEA4, MAGEB2, MAGEC1 and MAGEC2; and/or
(f) one or more of: APOL6, DTX3L, GBP1, IFI16, IFI27, IFI35, IFI6, IFIH1, IFIT1, IFIT2, IFIT3, IFITM1, IFITM2, IRF1, IRF9, ISG15, MX1, OAS1, OAS2, PARP9, PSMB9, STAT2, TMEM140 and TRIM21; and/or
(g) one or more of: PSMB8, PSMB9 and PSMB10; and/or
(h) IL-10; and or
(i) CD274; and/or
(j) PDCD1LG2.

The invention further provides the embodiment of such methods wherein the one or more target genes further comprises IFNG (NM_000619.2).

The invention further provides the embodiment of such methods wherein the one or more reference genes comprise one or more of: ABCF1, G6PD, NRDE2, OAZ1, POLR2A, SDHA, STK111P, TBC1D10B, TBP, and UBB.

The invention further provides the embodiment of such methods wherein a gene signature score is determined for the one or more target genes. In specific embodiments of the invention such gene signature score is determined from the raw RNA levels of each target gene by a process comprising:
(a) measuring the raw RNA levels for each target gene in one more cellular sample using a gene expression platform comprising a reference gene set of housekeeping genes,
(b) normalizing each of the measured raw RNA levels to the geometric mean of such housekeeping genes, and optionally further normalizing each RNA value to a standard,
(c) log transforming each normalized RNA value,
(d) multiplying each log transformed RNA value by a corresponding weight factor to generate a weighted RNA value, and
(e) adding the weighted RNA values, and optionally adding an adjustment factor constant, to generate a single gene signature score.

Preferably, the gene signature is determined using the target genes provided in Tables 6 and 12A-12G. In certain embodiments of the invention, the weight factors are those provided in Tables 6 and 12A-12G. In certain embodiments of the invention, an adjustment factor is added to each score. In particular embodiments, the adjustment factors are those provided in Tables 6 and 12B-12G.

The invention particularly, provides the embodiment of such methods wherein a gene signature score is determined for one or more of:
(a) the IFN Gamma Signaling Signature;
(b) the Tumor Inflammation Signature;
(c) the Myeloid Inflammation Signature;
(d) the Inflammatory Chemokine Signature;
(e) the MAGEs Signature;
(f) the IFN Downstream Signaling Signature;
(g) the Immunoproteasome Signature;
(h) the IL-10 Signature;
(i) the PD-L1 Signature; and/or
(j) the PD-L2 Signature.

The invention further provides the embodiment of such methods wherein a patient gene signature score that:
(a) is greater than the first quartile of scores for the gene signature calculated from the expression levels of one or more of the target genes in a population of individuals who are suffering from the hematologic malignancy; or
(b) is greater than the first quartile of scores for the gene signature calculated from the expression levels of one or more of the target genes in a population of individuals who did not successfully respond to a treatment for the hematologic malignancy that used a CD123×CD3 bispecific molecule; or
(c) has a log₂-fold change of at least about 0.4 relative to scores for the gene signature calculated from the expression levels of one or more of the target genes in a population of individuals who did not successfully respond to a treatment for the hematologic malignancy that used a CD123×CD3 bispecific molecule; or
(d) is within at least the first quartile of the scores for the gene signature calculated from the expression levels of one or more of the target genes in a population of individuals who successfully responded to a treatment for the hematologic malignancy that used a CD123×CD3 bispecific molecule, is indicative of a more favorable patient response to treatment with the CD123×CD3 bispecific molecule.

The invention further provides the embodiment of such methods wherein a patient gene signature score that:
(a) is greater than the second quartile for the gene signature calculated from the expression levels of one or more of the target genes in a population of individuals who are suffering from the hematologic malignancy; or
(b) is greater than the second quartile for the gene signature calculated from the expression levels of one or more of the target genes in a population of individuals who did not successfully respond to a treatment for the hematologic malignancy that used a CD123×CD3 bispecific molecule; or
(c) has a log₂-fold change of at least about 0.5 relative to scores for the gene signature calculated from the expression levels of one or more of the target genes in a population of individuals who did not successfully respond to a treatment for the hematologic malignancy that used a CD123×CD3 bispecific molecule; or
(d) is within at least the second quartile of the scores for the gene signature calculated from the expression levels of one or more of the target genes in a population of individuals who successfully responded to a treatment for the hematologic malignancy that used a CD123×CD3 bispecific molecule,
   is indicative of a more favorable patient response to treatment with the CD123×CD3 bispecific molecule.

The invention further provides the embodiment of such methods wherein a patient gene signature score that:
(a) is greater than the third quartile of scores for the gene signature calculated from the expression levels of one or more of the target genes in a population of individuals who are suffering from the hematologic malignancy; or
(b) is greater than the third quartile of scores for the gene signature calculated from the expression levels of one or more of the target genes in a population of individuals who did not successfully respond to a treatment for the hematologic malignancy that used a CD123×CD3 bispecific molecule; or
(c) has a log₂-fold change of at least about 0.6 relative to scores for the gene signature calculated from the expression levels of one or more of the target genes in a population of individuals who did not successfully respond to a treatment for the hematologic malignancy that used a CD123×CD3 bispecific molecule,
   is indicative of a more favorable patient response to treatment with the CD123×CD3 bispecific molecule.

The invention further provides the embodiment of such methods wherein:
(a) the gene signature is the IFN Gamma Signaling Signature, and a patient gene signature score of at least about 2.5 is indicative of a more favorable patient response to treatment with the CD123×CD3 bispecific molecule, and/or
(b) the gene signature is the Tumor Inflammation Signature, and a patient gene signature score of at least about 5.5 is indicative of a more favorable patient response to treatment with the CD123×CD3 bispecific molecule; and/or
(c) the gene signature is the IFN Downstream Signaling Signature, and a patient gene signature score of at least about 4.5 is indicative of a more favorable patient response to treatment with the CD123×CD3 bispecific molecule.

The invention further provides the embodiment of such methods wherein the gene signature is the IFN Gamma Signaling Signature, the Tumor Inflammation Signature, or the IFN Downstream Signaling Signature, and a patient gene signature score that:
(a) is greater than the first quartile of the scores for the gene signature calculated from the expression levels of one or more of the target genes in a population of individuals who are suffering from a hematologic malignancy; or
(b) is greater than the first quartile of the scores for the gene signature calculated from the expression levels of one or more of the target genes in a population of individuals who did not successfully respond to a treatment for the hematologic malignancy that used a CD123×CD3 bispecific molecule; or
(c) has a log₂-fold change of at least about 0.4 relative to the scores for the gene signature calculated from the expression levels of one or more of the target genes in a population of individuals who did not successfully respond to a treatment for the hematologic malignancy that used a CD123×CD3 bispecific molecule; or
(d) is within at least the first quartile of the scores for the gene signature calculated from the expression levels of one or more of the target genes in a population of individuals who successfully responded to a treatment for the hematologic malignancy that used a CD123×CD3 bispecific molecule,
   is indicative of a more favorable patient response to treatment with the CD123×CD3 bispecific molecule.

The invention further provides the embodiment of such methods wherein the gene signature is the IFN Gamma Signaling Signature, the Tumor Inflammation Signature, or the IFN Downstream Signaling Signature, and a patient gene signature score that:
(a) is greater than the second quartile of scores of the gene signature calculated from the expression levels of one or more of the target genes in a population of individuals who are suffering from the hematologic malignancy; or
(b) is greater than the second quartile of scores for the gene signature calculated from the expression levels of one or more of the target genes in a population of individuals who did not successfully respond to a treatment for the hematologic malignancy that used a CD123×CD3 bispecific molecule; or
(c) has a log₂-fold change of at least about 0.5 relative to scores for the gene signature calculated from the expression levels of one or more of the target genes in a population of individuals who did not successfully respond to a treatment for the hematologic malignancy that used a CD123×CD3 bispecific molecule; or
(d) is within at least the second quartile of the scores of the gene signature calculated from the expression levels of one or more of the target genes in a population of individuals who successfully responded to a treatment for a hematologic malignancy that used a CD123×CD3 bispecific molecule
   is indicative of a more favorable patient response to treatment with the CD123×CD3 bispecific molecule.

The invention also provides the embodiment of such methods wherein a patient that exhibits a gene expression signature that is characteristic of an immune-enriched and IFN gamma-dominant tumor microenvironment is indicative of a more favorable patient response to treatment with the CD123×CD3 bispecific molecule.

The invention further provides the embodiment of such methods wherein the CD123×CD3 bispecific molecule is a bispecific antibody or a bispecific molecule comprising an scFv.

The invention further provides the embodiment of such methods wherein the CD123×CD3 bispecific molecule is JNJ-63709178, XmAb14045 or APVO436.

The invention further provides the embodiment of such methods wherein the CD123×CD3 bispecific molecule is a covalently bonded bispecific diabody having two, three, or four polypeptide chains.

The invention further provides the embodiment of such methods wherein the CD123×CD3 bispecific molecule is a diabody that comprises:
(a) a first polypeptide chain having the amino acid sequence of SEQ ID NO:21; and
(b) a second polypeptide chain having the amino acid sequence of SEQ ID NO:23; and
   wherein the first and the second polypeptide chains are covalently bonded to one another by a disulfide bond.

The invention further provides the embodiment of such methods wherein the hematologic malignancy of such patient is selected from the group consisting of: acute myeloid leukemia (AML), chronic myelogenous leukemia (CIVIL), blastic crisis of CML, Abelson oncogene-associated with CIVIL (Bcr-ABL translocation), myelodysplastic syndrome (MDS), acute B lymphoblastic leukemia (B-ALL), acute T lymphoblastic leukemia (T-ALL), chronic lymphocytic leukemia (CLL), Richter's syndrome, Richter's transformation of CLL, hairy cell leukemia (HCL), blastic plasmacytoid dendritic cell neoplasm (BPDCN), non-Hodgkin's lymphoma (NHL), including mantle cell lymphoma (MCL) and small lymphocytic lymphoma (SLL), Hodgkin's lymphoma, systemic mastocytosis, and Burkitt's lymphoma.

The invention further provides the embodiments of such methods wherein the hematologic malignancy of such patient is AML, MDS, BPDCN, or T-ALL.

The invention further provides the embodiment of such methods wherein the hematologic malignancy of such patient is refractory to chemotherapy (CTX), such as being refractory to cytarabine/anthracycline-based cytotoxic chemotherapy or refractory to hypomethylating agents (HMA) chemotherapy.

The invention further provides the embodiment of such methods that further comprises determining the level expression of CD123 of blast cells (cancer cells) as compared to a corresponding baseline level CD123 expressed by normal peripheral blood mononuclear cells (PBMCs).

The invention further provides the embodiment of such methods wherein the level of expression is determined by measuring the cell surface expression of CD123. The invention further provides the embodiment of such methods wherein the cell surface expression of CD123 is increased by at least about 20% relative to a baseline level of expression. The invention further provides the embodiment of such methods wherein the increase in CD123 expression renders the patient more responsive to treatment with the CD123×CD3 bispecific molecule.

The invention further provides the embodiment of such methods wherein the effective dosage of the CD123×CD3 bispecific molecule is selected from the group consisting of 30, 100, 300, and 500 ng/kg patient weight/day.

The invention further provides the embodiment of all of the above-described methods wherein the treatment dosage is administered as a continuous infusion. The invention further provides the embodiment of such methods wherein the treatment dosage is 30 ng/kg/day administered by continuous infusion for 3 days followed by a treatment dosage of 100 ng/kg/day administered by continuous infusion for 4 days. The invention further provides the embodiment of such methods wherein the treatment dosage further comprises administration of 500 ng/kg/day administered by continuous infusion.

The invention further provides the embodiment of all of the above-described methods wherein the patient is a human patient.

### BRIEF DESCRIPTION OF THE DRAWINGS

FIGS. 1A-1C illustrate the overall structure of exemplary diabody molecules. FIG. 1A provides the structure of the first and second polypeptide chains of a two chain CD123×CD3 bispecific diabody (“DART-A” also known as flotetuzumab) having two epitope-binding domains, Heterodimer-Promoting Domains and a cysteine containing linker. FIGS. 1B-1C provide the overall structure of a CD123×CD3 bispecific diabody having two epitope-binding domains composed of three polypeptide chains. Two of the polypeptide chains possess a CH2 and CH3 Domain, such that the associated chains form all or part of an Fc Domain. The polypeptide chains comprising the VL and VH Domain further comprise a Heterodimer-Promoting Domain and a linker. A cysteine residue may be present in a linker (FIGS. 1A and 1B) and/or in the Heterodimer-Promoting Domain (FIG. 1C). VL and VH Domains that recognize the same epitope are shown using the same shading or fill pattern.

FIG. 2 illustrates unsupervised hierarchical clustering of the 46 IO 360 signatures or cell types generated from the baseline bone marrow biopsy obtained from patients that had had a refractory response to conventional chemotherapy (e.g., patient refractory response to a regimen of treatment with cytarabine given in conjunction with daunorubicin (7+3 induction therapy (Ref CTX)) or patients that had a refractory response to a regimen of treatment with the hypomethylating agents decitabine and azacitidine (Ref HMA and including patients with secondary AML), and patients that relapsed (Relapse) all prior to flotetuzumab treatment. Also indicated are the patients' responses to CD123×CD3 bispecific binding molecule therapy with flotetuzumab. Such responses were annotated as being either an anti-leukemic response (Anti-leuk (A)), which included patients exhibiting a complete response (CR), a complete response with incomplete hematological improvement (CRi), a morphologic leukemia-free state (MLF), other anti-leukemic benefit (OB), or a partial response (PR)), or as non-responding (NR, which included progressive disease/treatment failure (PD), and stable disease (SD)). Each IO 360 signature score was rescaled within the score for this cohort to a −3 to +3 scale to facilitate comparison across signatures. Immune Exhausted and Immune Enriched gene signatures are boxed in Cluster 2 and Cluster 3 columns, respectively.

FIGS. 3A-3O show that chemo- and HMA-refractory patients have different expression of multiple gene signatures. In particular, the gene expression profiles of Relapsed patients display features of immune depletion while the profiles of HMA-refractory (including HMA-refractory and secondary AML) patients displayed features of immune exhaustion and adaptive immune resistance, including upregulation of TIGIT, PD-L1 and Treg gene signatures together with a trend toward increasing gene signatures associated with exhausted CD8 T cells compared to CTX-refractory patients. FIG. 3A is a forest plot of the fold change differences between Relapsed patients change from all refractory (CTX and HMA). FIG. 3B is a forest plot of the fold change differences between HMA-refractory patients change from Relapse; FIG. 3C is a forest plot of the fold change differences between HMA-refractory patients change from CTX-refractory patients. Cluster 2 Immune Exhausted (C2) and Cluster 3 Immune Enriched (C3) gene signatures are indicated in FIGS. 3A and 3C. The Myeloid (FIG. 3D), Macrophage (FIG. 3E), Neutrophil (FIG. 3F), B-cell (FIG. 3G), IFN gamma (IFN-γ, FIG. 3H), PD-L1 (FIG. 3I), TIGIT (FIG. 3J), CTLA-4 (FIG. 3K), Th1 (FIG. 3L), CTL (FIG. 3M), CD8 T cell (FIG. 3N), and Cytotoxicity (FIG. 3O) gene signature scores are plotted for Immune Depleted (Depl.), Immune Enriched (Enriched), and Immune Exhausted (Exh.) profiles.

FIG. 4 shows the percent change (relative to baseline) in bone marrow blasts from 25 patients (Relapse (RL) patients, patients that were CTX-Refractory (CTx), and patients that were HMA-Refractory (HMA)) after CD123×CD3 bispecific binding molecule therapy and their response to such therapy (CR, Complete Response; mCR, molecular CR; CRi, Complete Response with incomplete hematological improvement; MLF, Morphologic Leukemia-free state; PR, Partial Response; SD, Stable Disease; PD, Progressive Disease/Treatment Failure).

FIGS. 5A-5C show that the IFN Gamma Signaling Signature is increased at baseline in Responders to flotetuzumab, and that the IFN Gamma Signaling Signature is therefore predictive of a positive response to CD123×CD3 bispecific binding molecule therapy. FIG. 5A is a forest plot of the baseline fold change differences between OR patients and NR patients showing that the IFN Gamma Signaling Signature was increased in baseline samples in OR patients (Immune Exhausted (C2) and Immune Enriched (C3) gene signatures are indicated). The Tumor Inflammation Signature and IFN Downstream Signature were also seen to increase. FIG. 5B shows the distribution of IFN Gamma Signaling Signature scores in NR and OR populations of patients (2^{nd} AML; Ref CTX: refractory to CTX; Ref HMA: refractory to HMA, Relapse: primary relapse). FIG. 5C shows ROC curves showing predictive performance of the baseline IFN Gamma Signaling Signature score with an AUC=0.819.

FIG. 6 shows the expression of gene signatures associated with Cytotoxic cells, or with CD8+ T cells, as examined in RNA from bone marrow samples, either pre-treatment (“Base”) or from bone marrow samples after a first cycle of treatment with flotetuzumab (“Cycle 1”).

FIG. 7 shows the expression of CD123 in patient populations that were either refractory to chemotherapy, in relapse, refractory to HMA, or in HMA failure.

FIG. 8 shows the correlation between the level of expression of PD-L1 in patient AML blasts at baseline (BL) and whether the patients were early progressors or responders to CD123×CD3 bispecific binding molecule therapy. Data is expressed as mean+distribution.

FIG. 9 illustrates unsupervised hierarchical clustering of 48 IO 360 signatures or cell types generated from the baseline bone marrow biopsy obtained from patients that had had a primary refractory response to conventional chemotherapy (P), and patients that relapsed (R) all prior to flotetuzumab treatment. Also indicated are the patients' responses to CD123×CD3 bispecific binding molecule therapy with flotetuzumab. Such responses were annotated as being either an anti-leukemic response (A, which included patients exhibiting a complete response (CR), a complete response with incomplete hematological improvement (CRi), a morphologic leukemia-free state (MLF), other anti-leukemic benefit (OB), or a partial response (PR)), or as non-responding (N, which included progressive disease/treatment failure (PD), and stable disease (SD)). Each IO 360 signature score was rescaled within the score for this cohort to a −3 to +3 scale to facilitate comparison across signatures. Stratification into Immune-infiltrated and Immune-depleted clusters is indicated.

FIG. 10 is a forest plot of the baseline fold-change differences of relapsed and refractory patients between those exhibiting an anti-leukemic response (OR) and non-responders (NR) to CD123×CD3 bispecific binding molecule therapy with flotetuzumab, showing that numerous signatures were increased in baseline samples from responders including: the IFN Gamma Signaling Signature, IFN Downstream Signature, and Tumor Inflammation Signature (each boxed). The gene signatures which make up the IFN Dominant Module are starred and are also increased in baseline samples from responders.

FIGS. 11A-11D show the score distribution of several gene signatures and the IFN module in refractory (Refr.) and relapsed (Rel.) patients, OR patients are indicated with large open circles, NR patients are indicated with small solid dots. Comparisons were performed with the Mann-Whitney U test for paired data. **P<0.01. FIG. 11A shows the distribution of the IFN Gamma Signaling Signature scores. FIG. 11B shows the distribution of the IFN Downstream Signaling Signature scores. FIG. 11C shows the distribution of the Tumor Inflammation Signature (TIS) scores. FIG. 11D shows the distribution of the IFN Dominant Module (IFN module) scores.

FIGS. 12A-12J shows the score distribution of the scores of the nine gene signatures that make up the IFN Dominant Module and the Tumor Inflammation Signature (TIS) in non-responders (NR) and responding patients (patients having an anti-leukemic response)(OR). FIG. 12A shows the IFN Gamma Signaling Signature scores; FIG. 12B shows the IFN Downstream Signature scores; FIG. 12C shows the Myeloid Inflammation Signature scores; FIG. 12D the Immunoproteasome Signature scores; FIG. 12E shows the Inflammatory Chemokines Signature scores; FIG. 12F shows the MAGEs Signature scores; FIG. 12G shows the PD-L1 Signature scores; FIG. 12H the PD-L2 Signature scores; FIG. 12I the IL10 Signature scores; FIG. 12J the Tumor Inflammation Signature (TIS) scores.

FIGS. 13A-13K shows ROC curves showing predictive performance of the baseline scores for the nine gene signatures that make up the IFN Dominant Module, the Tumor Inflammation Signature (TIS), and the IFN Dominant Module for the group of 30 refractory/relapsed patients. FIG. 13A shows the ROC curve for the IFN Gamma Signaling Signature scores with an AUC=0.750. FIG. 13B shows the ROC curve for the IFN Gamma Downstream Signaling Signature scores with an AUC=0.755. FIG. 13C shows the ROC curve for the Myeloid Inflammation Signature scores with an AUC=0.69. FIG. 13D shows the ROC curve for the Immunoproteasome Signature scores with an AUC=0.505. FIG. 13E shows the ROC curve for the Inflammatory Chemokines Signature scores with an AUC=0.764. FIG. 13F shows the ROC curve for the MAGEs Signature score with an AUC=0.736. FIG. 13G shows the ROC curve for the PD-L1 Signature scores with an AUC=0.699. FIG. 13H shows the ROC curve for the PD-L2 Signature score with an AUC=0.727). FIG. 13I shows the ROC curve for the IL10 Signature scores with an AUC=0.745). FIG. 13J shows ROC curve for the TIS scores with an AUC=0.852. FIG. 13K shows ROC curves for the IFN Dominant Module scores with an AUC=0.806.

FIGS. 14A-14D show the score distribution of the Tumor Inflammation (TIS, FIG. 14A), IFN Gamma Signaling (FIG. 14B), Antigen Processing Machinery (APM, FIG. 14C), and PD-L1 (FIG. 14D) gene signatures as examined in RNA from bone marrow samples, either pre-treatment (“Pre”) or from bone marrow samples after a first cycle of treatment with flotetuzumab (“Post-C1”), OR patients are indicated with large open circles, NR patients are indicated with small solid dots. Comparisons were performed with the Mann-Whitney U test for paired data. Pre=baseline. C1=cycle 1. **P<0.01. ***P<0.001.

### DETAILED DESCRIPTION OF THE INVENTION

The present invention is directed to a method of treating a hematologic malignancy such as acute myeloid leukemia (AML) or myelodysplastic syndrome (MDS), including hematologic malignancies that are refractive to chemotherapeutic and/or hypomethylating agents. The method concerns administering a CD123×CD3 bispecific binding molecule to a patient in an amount effective to stimulate the killing of cells of said hematologic malignancy in said patient. The present invention is additionally directed to the embodiment of such method in which a cellular sample from the patient evidences an expression of one or more target genes that is increased relative to a baseline level of expression of such genes, for example, a baseline level of expression of such genes in a reference population of individuals who are suffering from the hematologic malignancy, or with respect to the level of expression of a reference gene.

As indicated above, chemotherapy resistance and relapse remain significant sources of mortality for children and adults with acute myeloid leukemia (AML). Receiving conventional chemotherapy, only 26.9% of patients are expected to survive beyond 5 years.

The therapeutic approach in patients with acute myeloid leukemia (AML) has not changed substantially in more than 30 years. The standard front line therapy is a two-drug regimen of cytarabine given in conjunction with daunorubicin (the so-called 7+3 induction therapy, abbreviated herein as “CTX”). The hypomethylating agents (abbreviated herein as “HMA”) decitabine and azacitidine are commonly administered to older patients or to those considered unfit for the CTX regimen. However, estimates from the literature indicate that up to 45% of patients are refractory to standard frontline chemotherapy. Further intensification of conventional cytotoxic chemotherapy has been deemed to not be feasible due to the severity of acute and long-term side effects upon normal tissues commonly induced by these drugs (Tasian, S. K. (2018 “Acute Myeloid Leukemia Chimeric Antigen Receptor T-Cell Immunotherapy: How Far Up The Road Have We Traveled?,” Ther. Adv. Hematol. 9(6):135-148; Przespolewski, A. et al. (2018) “Advances In Immunotherapy For Acute Myeloid Leukemia” Future Oncol. 14(10):963-978; Shimabukuro-Vornhagen, A. et al. (2018) “Cytokine Release Syndrome,” J. Immunother. Cancer. 6(1):56 pp. 1-14; Milone, M. C. et al. (2018) “The Pharmacology of T Cell Therapies,” Mol. Ther. Methods Clin. Dev. 8:210-221; Dhodapkar, M. V. et al. (2017) “Hematologic Malignancies: Plasma Cell Disorders,” Am. Soc. Clin. Oncol. Educ. Book. 37:561-568; Kroschinsky, F. et al. (2017) “New Drugs, New Toxicities: Severe Side Effects Of Modern Targeted And Immunotherapy Of Cancer And Their Management,” Crit. Care 14; 21(1):89).

Bispecific antibodies that engage T cells stimulate the release of proinflammatory cytokines. Such cytokines can increase anti-leukemia efficacy by direct cytotoxicity and by activation and recruitment of immune cells into the tumor site (Hoseini, S. S. et al. (2107) “Acute Myeloid Leukemia Targets For Bispecific Antibodies,” Blood Cancer Journal 7:e522, doi:10.1038/bcj.2017.2; pp. 1-12. In particular, treatment with flotetuzumab, a CD123×CD3 bispecific binding molecule, is being tested in a Phase 1/2 study of relapsed/refractory (“R/R”) AML. Despite the great potential of immunotherapy to selectively target the cancer cells causing hematologic malignancies (see, e.g., Koch, J. et al. (2017) “Recombinant Antibodies to Arm Cytotoxic Lymphocytes in Cancer Immunotherapy,” Transfus. Med. Hemother. 44:337-350; Lichtenegger, F. S. et al. (2017) “Recent Developments In Immunotherapy Of Acute Myeloid Leukemia,” J. Hematol. Oncol. 10:142, pp. 1-20), efforts to employ bispecific binding molecules that are capable of targeting a T cell to the location of a hematologic malignancy have not been fully successful.

The discovery of new treatment strategies, including immunotherapy, thus remains a priority. It has previously been reported that AML patients with an immune-enriched and IFN gamma-dominant tumor microenvironment (“TME”) experience significantly shorter relapse-free survival, suggesting refractoriness to standard induction chemotherapy (Vadakekolathu, J. et al. (2017) “Immune Gene Expression Profiling in Children and Adults with Acute Myeloid Leukemia Identifies Distinct Phenotypic Patterns,” Blood 130:3942A).

As used herein, the term “gene expression signature” is intended to denote a pattern of gene expression of a group of genes that is characteristic of a particular cell type and/or biological process (see, e.g., Stenner, F. et al. (2018) “Cancer Immunotherapy and the Immune Response in Follicular Lymphoma,” Front. Oncol. 8:219 doi: 10.3389/fonc.2018.00219, pages 1-7; Cesano, A. et al. (2018) “Bringing The Next Generation Of Immuno-Oncology Biomarkers To The Clinic,” Biomedicines 6(14) doi: 10.3390/biomedicines6010014, pages 1-11; Shrestha, G. et al. (2016) “The Value Of Genomics In Dissecting The RAS-Network And In Guiding Therapeutics For RAS-Driven Cancers,” Semin. Cell Dev. Biol. 58:108-117; Gingras, I. et al. (2015) “CCR 20th Anniversary Commentary: Gene-Expression Signature in Breast Cancer—Where Did It Start and Where Are We Now?,” Clin. Cancer Res. 21(21):4743-4746; Eberhart, C. G. (2011) “Molecular Diagnostics In Embryonal Brain Tumors,” Brain Pathol. 21(1):96-104; Baylin, S. B. (2009) “Stem Cells, Cancer, And Epigenetics,” StemBook, ed. THE STEM CELL RESEARCH COMMUNITY, StemBook, doi/10.3824/stembook.1.50.1, pages 1-14; Asakura, M. et al. (2009) “Global Gene Expression Profiling In The Failing Myocardium,” Circ. J. 73 (9): 1568-1576; Shaffer, A. L. et al. (2001) “Signatures Of The Immune Response,” Immunity 15(3):375-385; Staudt, L. M. et al. (2005) “The Biology Of Human Lymphoid Malignancies Revealed By Gene Expression Profiling,” Adv. Immunol. 87:163-208). An observed gene expression signature, and/or changes in that signature resulting from altered (or unaltered) biological process(es), can be used to assess the presence, nature and/or severity of a pathogenic medical condition.

A central aspect of the present invention relates to the recognition that the presence of IFN gamma-dominant AML tumor microenvironments (“TMEs”), in contrast to predicting resistance to standard chemotherapy, predicts a favorable response to therapy employing CD123×CD3 bispecific binding molecules, including therapy employing the CD123×CD3 bispecific binding molecule, flotetuzumab. The invention derives in part from the recognition that certain sub-populations of patients having a refractory hematologic malignancy (e.g., an acute myeloid leukemia) are particularly amenable to treatment with the CD123×CD3 bispecific binding molecules (e.g., flotetuzumab). Members of this sub-population can be readily identified by their ability to exhibit a gene expression signature that is characteristic of the presence of an immune-enriched and IFN gamma-dominant tumor microenvironment.

### I. Identification of Patient Populations Particularly Suitable for Treatment with the CD123×CD3 Bispecific Binding Molecules of the Invention

A. Methods for Determining “Gene Expression Signatures”

In order to determine whether a patient exhibits a gene expression signature that is characteristic of the presence of an immune-enriched and IFN gamma-dominant tumor microenvironment, so as to be thereby identified as being particularly amenable for the treatment of a hematologic malignancy using the methods and compositions of the present invention, an RNA sample from a cellular sample obtained from a patient is evaluated to determine whether it evidences increased expression of one or more “target” genes whose expression correlates with such a signature. Such evaluation may make use of pre-existing detection and/or measurements of gene expression or may incorporate the step(s) of detecting and/or measuring such gene expression. As used herein, the term “cellular sample” refers to a sample that contains cells or an extract of cells.

Any cellular sample may be employed as a source of RNA or protein for use in determining whether a patient exhibits a gene expression signature that is characteristic of the presence of an immune-enriched and IFN gamma-dominant tumor microenvironment. Preferably, however, such gene expression comparisons are conducted using RNA obtained from a bone marrow (BM) sample or from a blood sample or a sample of blast cells (cancer cells) of the patient or of a population of donors. Where RNA is obtained from such cells of a population of donors to provide a baseline expression level, the average of the employed expression levels may be used (e.g., a geometric mean may be employed). A number of different reference populations may be used for such gene expression comparisons. In particular embodiments, the expression level of at least one target gene exhibited by a patient is compared to the expression level of such target gene exhibited in: a population of individuals who are suffering from a hematologic malignancy; a population of individuals who were suffering from such hematologic malignancy at the time such reference expression level was determined and who did not successfully respond to a treatment for a hematologic malignancy (i.e., a population of individuals who did not successfully respond to a treatment for a hematologic malignancy using a CD123×CD3 bispecific molecule); and/or a population of individuals who were suffering from such hematologic malignancy at the time such reference expression level was determined and who were thereafter successfully treated for a hematologic malignancy using the methods and compositions of the present invention (i.e., a population of individuals who successfully responded to a treatment for a hematologic malignancy using a CD123×CD3 bispecific molecule). Where the comparator population is a population of individuals who are suffering from a hematologic malignancy such population preferably includes individuals who are suffering from the same hematological malignancy as the patient. Such population may include individuals that have relapsed after prior treatment with a chemotherapeutic agent and/or that were refractory to treatment with a chemotherapeutic agent (i.e., primary refractory). Where the comparator population is a population of individuals who successfully, or unsuccessfully responded to a treatment for a hematologic malignancy CD123×CD3 bispecific molecule such population preferably includes individuals who are suffering from the same hematological malignancy as the patient.

As used herein, the expression of a gene is said to be “increased” if, relative to a baseline or other comparator (e.g., expression of such gene in a population), its expression is at least about 10% greater, at least about 20% greater, at least about 30% greater, at least about 40% greater, at least about 50% greater, at least about 60% greater, at least about 70% greater, at least about 80% greater, at least about 90% greater, at least about 1.5-fold greater, at least about 2-fold greater, at least about 2.5-fold greater, at least about 3-fold greater, at least about 3.5-fold greater, at least about 4-fold greater, at least about 4.5-fold greater, at least about 5-fold greater, at least about 5.5-fold greater, at least about 6-fold greater, at least about 6.5-fold greater, at least about 7-fold greater, at least about 7.5-fold greater, at least about 8-fold greater, at least about 8.5-fold greater, at least about 9-fold greater, at least about 10-fold greater. Such increases can be alternatively described in terms of “log₂-fold changes.” With respect to increases in expression, a log₂-fold change of 0.4 is equivalent to about 30% greater expression a log₂-fold change of 0.5 is equivalent to about 40% greater expression; a log₂-fold change of 0.6 is equivalent to about 50% greater expression; a log₂-fold change of 0.7 is equivalent to about 60% greater expression; a log₂-fold change of 0.8 is equivalent to about 70% greater expression; a log₂-fold change of 0.9 is equivalent to about 90% greater expression; a log₂-fold change of 1 is equivalent to a 2-fold increase; a log₂-fold change of 1.5 is equivalent to a 2.8-fold increase; a log₂-fold change of 2 is equivalent to a 4-fold increase; a log₂-fold change of 2.5 is equivalent to a 5.7-fold increase; a log₂-fold change of 3 is equivalent to an 8-fold increase; a log 2-fold change of 3.5 is equivalent to an 11.3-fold increase; a log₂-fold change of 4 is equivalent to a 16-fold increase, etc. Log₂ fold changes are commonly used when comparing counts to array data and are also appropriate for t-tests.

Alternatively, such increases are described in terms of a “gene signature score” wherein the expression of each of a cluster of target genes is measured, normalized to one or more housekeeping genes and/or internal standards, log transformed, weighted and summed to generate a single gene signature score. Methods for calculating such scores are known in the art and specific methods are provided herein (see, Example 1 below).

As used herein the expression of a gene signature (e.g., an IFN Gamma Signaling Signature) is said to be “increased” if the gene signature score is at least about 2, or at least about 2.5, or at least about 3.0, or at least about 3.5, or at least about 4, or at least about 4.5, or at least about 5, or is at least about 5, or at least about 5.5, or at least about 5.5, or at least about 6, or is greater than about 6.5.

A gene signature score of a patient is also said to be “increased” if it is greater than the first quartile of gene signature scores (i.e., greater than the bottom 25%), greater than the second quartile of gene signature scores (i.e., greater than the lower 50%), greater than the third quartile of gene signature scores (i.e., greater than the lower 75%), greater than 85%, greater than 90%, or greater than 95% of the gene signature scores calculated from the expression levels of such target genes in a population of individuals who are suffering from a hematologic malignancy.

A gene signature score of a patient is also said to be “increased” if it is greater than the first quartile of gene signature scores (i.e., greater than the bottom 25%), greater than the second quartile of gene signature scores (i.e., greater than the lower 50%), greater than the third quartile of gene signature scores (i.e., greater than the lower 75%), greater than 85%, greater than 90%, or greater than 95% of the gene signature scores calculated from the expression levels of such target genes in a population of individuals who did not successfully respond to a treatment for a hematologic malignancy (e.g., a population of individuals who did not successfully respond to a treatment for a hematologic malignancy CD123×CD3 bispecific molecule).

A gene signature score of a patient is also said to be “increased” if it has a log₂-fold change of at least about 0.4, or at least about 0.5, or at least about 0.6, or greater, relative to the gene signature scores calculated from the expression levels of such target genes in a population of individuals who did not successfully respond to a treatment for a hematologic malignancy (e.g., a population of individuals who did not successfully respond to a treatment for a hematologic malignancy CD123×CD3 bispecific molecule).

A gene signature score of a patient is also said to be “increased” if it is within at least the first quartile of gene signature scores (i.e., within the bottom 25%), and more preferably, within at least the second quartile (i.e., between the bottom 25% and 50%), within at least the third quartile (i.e., between the bottom 50% and 75%), greater than 85%, greater than 90%, or greater than 95% of the gene signature scores calculated from the expression levels of such target genes in a population of individuals who have previously been successfully treated for a hematologic malignancy using the methods and compositions of the present invention (e.g., a population of individuals who successfully responded to a treatment for a hematologic malignancy using a CD123×CD3 bispecific molecule).

A finding of an increased gene signature score is indicative of a more favorable patient response to treatment for hematologic malignancy with the CD123×CD3 bispecific molecules of the present invention.

In one embodiment, a patient is identified as exhibiting a gene expression signature that is characteristic of the presence of an immune-enriched and IFN gamma-dominant tumor microenvironment and to thus be particularly amenable to the treatment of hematologic malignancy using the methods and compositions of the present invention by determining whether the expression of a target gene is “increased” relative to the baseline level of its expression in the patient being evaluated when such patient was healthy, or before such patient had received a diagnosis of hematologic malignancy, or relative to the expression of that gene at a time during such patient's course of a chemotherapy treatment regimen or during such patient's course of a treatment regimen involving a CD123×CD3 bispecific binding molecule.

In a second embodiment, a patient is identified as exhibiting a gene expression signature that is characteristic of the presence of an immune-enriched and IFN gamma-dominant tumor microenvironment and as thus being particularly amenable to the treatment of hematologic malignancy using the methods and compositions of the present invention by comparing the level of expression of one or more target gene(s) to the averaged or weighted baseline level of expression of such target gene(s) in a population of individuals who are suffering from a hematologic malignancy. A target gene whose expression is greater than such an averaged or weighted baseline level is said to exhibit an “increased” level of expression, and the methods and compositions of the present invention are particularly suitable for use in treating hematologic malignancy in such patients. For example, the methods and compositions of the present invention are particularly suitable for use in patients who exhibit an “increased” level of target gene(s) expression that is greater than the first quartile (i.e., greater than the bottom 25%) of the expression levels of such target gene(s) in a population of individuals who are suffering from a hematologic malignancy. The methods and compositions of the present invention are particularly suitable for use in patients who exhibit an “increased” level of target gene(s) expression that is greater than the second quartile (i.e., greater than the bottom 50%) of the expression levels of such target gene(s) in a population of individuals who are suffering from a hematologic malignancy. The methods and compositions of the present invention are particularly suitable for use in patients who exhibit an “increased” level of target gene(s) expression that is greater than the third quartile (i.e., greater than the bottom 75%) of the expression levels of such target gene(s) in a population of individuals who are suffering from a hematologic malignancy. The methods and compositions of the present invention are particularly suitable for use in patients who exhibit an “increased” level of target gene(s) expression that is greater than 85%, greater than 90%, or greater than 95% of the expression levels of such target gene(s) in a population of individuals who are suffering from a hematologic malignancy.

In a third embodiment, a patient is identified as exhibiting a gene expression signature that is characteristic of the presence of an immune-enriched and IFN gamma-dominant tumor microenvironment and as thus being particularly amenable to the treatment of hematologic malignancy using the methods and compositions of the present invention by comparing the level of expression of one or more target gene(s) to the averaged or weighted baseline level of expression of such target gene(s) in a population of individuals who have previously been unsuccessfully treated for a hematologic malignancy using the methods and compositions of the present invention (e.g., a population of individuals who did not successfully respond to a treatment for a hematologic malignancy using a CD123×CD3 bispecific molecule). A target gene whose expression is equal or greater than such an averaged or weighted baseline level is said to exhibit an “increased” level of expression, and the methods and compositions of the present invention are particularly suitable for use in treating hematologic malignancy in such patients. The methods and compositions of the present invention are particularly suitable for use in patients who exhibit an “increased” level of target gene(s) expression that is greater than the first quartile (i.e., greater than the bottom 25%) of the expression levels of such target gene(s) in such population of unsuccessfully-treated individuals. The methods and compositions of the present invention are particularly suitable for use in patients who exhibit an “increased” level of target gene(s) expression that is greater than the second quartile (i.e., greater than the bottom 50%) of the expression levels of such target gene(s) in such population of unsuccessfully-treated individuals. The methods and compositions of the present invention are particularly suitable for use in patients who exhibit an “increased” level of target gene(s) expression that is greater than the third quartile (i.e., greater than the bottom 75%) of the expression levels of such target gene(s) in such population of unsuccessfully-treated individuals. The methods and compositions of the present invention are particularly suitable for use in patients who exhibit an “increased” level of target gene(s) expression that is greater than 85%, greater than 90%, or greater than 95% of the expression levels of such target gene(s) in such population of unsuccessfully-treated individuals.

In a fourth embodiment, a patient is identified as exhibiting a gene expression signature that is characteristic of the presence of an immune-enriched and IFN gamma-dominant tumor microenvironment and as thus being particularly amenable to the treatment of hematologic malignancy using the methods and compositions of the present invention by comparing the level of expression of one or more target gene(s) to the averaged or weighted baseline level of expression of such target gene(s) in a population of individuals who have previously been successfully treated for a hematologic malignancy using the methods and compositions of the present invention (e.g., a population of individuals who successfully responded to a treatment for a hematologic malignancy using a CD123×CD3 bispecific molecule). A target gene whose expression is equal or greater than such an averaged or weighted baseline level is said to exhibit an “increased” level of expression, and the methods and compositions of the present invention are particularly suitable for use in treating hematologic malignancy in such patients. The methods and compositions of the present invention are particularly suitable for use in patients who exhibit an “increased” level of target gene(s) expression that is within at least the first quartile (i.e., within the bottom 25%) of the expression levels of such target gene(s) in such population of successfully-treated individuals. The methods and compositions of the present invention are particularly suitable for use in patients who exhibit an “increased” level of target gene(s) expression that is within at least the second quartile (i.e., between the bottom 25% and 50%) of the expression levels of such target gene(s) in such population of successfully-treated individuals. The methods and compositions of the present invention are particularly suitable for use in patients who exhibit an “increased” level of target gene(s) expression that is within at least the third quartile (i.e., between the bottom 50% and 75%) of the expression levels of such target gene(s) in such population of successfully-treated individuals. The methods and compositions of the present invention are even more particularly suitable for use in patients who exhibit an “increased” level of target gene(s) expression that is within at least the fourth quartile (i.e., above the bottom 75%) of the expression levels of such target gene(s) in such population of previously-treated individuals.

However, it is preferred to determine whether a target gene's expression is “increased” by comparing the level of its expression to the level of expression of one or more genes that are not associated with disease or that do not exhibit increased expression as a consequence of a disease state (“reference” genes). Because reference genes are often expressed at different levels, the geometric mean of the reference genes' expression can be utilized to calculate scaling factors. A geometric mean is obtained by multiplying each gene per sample value in a data set and then taking the n^{th} root (where n is the count of numbers in the set) of the resulting product. A geometric mean is similar to an arithmetic mean, in that it indicates the central tendency of a set of numbers. However, unlike an arithmetic mean, the geometric mean is less sensitive to variation in the magnitude of count levels between probes. To compare biological signatures across a cohort of samples the geometric mean from a set of “reference” gene(s) may be used to normalize individual samples across a data set in order for comparisons between biological genes to be made independent of differences due to technical variation such as sample mass input and sample quality.

Preferred “reference” genes are constitutively expressed at the same level in normal and malignant cells. Housekeeping genes (Eisenberg, E. et al. (2003) “Human Housekeeping Genes Are Compact,” Trends in Genetics. 19(7):362-365; kon Butte, A. J. et al. (2001) “Further Defining Housekeeping, Or “Maintenance,” Genes Focus On ‘A Compendium Of Gene Expression In Normal Human Tissues’,” Physiol. Genomics. 7(2):95-96; Zhu, J. et al. (2008) “On The Nature Of Human Housekeeping Genes,” Trends in Genetics 24(10):481-484; Eisenberg, E. et al. (2013) “Human Housekeeping Genes, Revisited,” Trends in Genetics. 29(10):569-574) such as genes required for the maintenance of basic cellular functions are a preferred class of reference genes.

In a further embodiment, a determination of whether a patient is particularly suitable for treatment with CD123×CD3 binding molecule therapy further comprises:
(a) evaluating the level of expression of CD123 in the patient's blast cells (cancer cells). Such level may be compared to the corresponding baseline level CD123 relative to the expression of CD123 in such patient's blast cells at a time prior to or during such patient's course of a chemotherapy treatment regimen, or relative to the expression of CD123 in the blast cells of a population of individuals that have relapsed or are refractory to HMA therapy. A finding of increased expression of CD123 is further indicative of the patient's suitability for receiving CD123×CD3 binding molecule therapy for a hematologic malignancy. The determination of CD123 expression may be accomplished by assessing the presence of CD123-encoding mRNA, or by assessing the presence of CD123 in a cellular lysate or extract. Alternatively, the determination of CD123 expression may be accomplished by assessing the presence of CD123 molecules arrayed on the cell-surface of CD123-expressing cells (e.g., blast cells). Increased expression (e.g., at least a 10% increase in expression, at least a 15% increase in expression, at least a 20% increase in expression, at least a 25% increase in expression, at least a 30% increase in expression, or at least a 40% increase in expression) of CD123 as so determined is indicative of the patient's suitability for receiving CD123×CD3 binding molecule therapy for a hematologic malignancy.
and/or
(b) evaluating the level of expression of PD-L1 in the patient's blast cells (cancer cells). In certain embodiments, the level of PD-L1 expression is evaluated across a sample of the patient's blast cells such that the percent of blast cells expressing PD-L1 is evaluated. In other embodiments, the level of PD-L1 expression in the patients blast cells is compared to the relative to the expression of PD-L1 in such patient's blast cells at a time during such patient's course of a chemotherapy treatment regimen. Thus, the level of expression of PD-L1 in the patient's cells may be assessed prior to any administration of a CD123×CD3 binding molecule and/or after such administration. A finding of low expression of PD-L1 is indicative of the patient's suitability for receiving CD123×CD3 binding molecule therapy for a hematologic malignancy. A finding of high expression of PD-L1 is indicative of the patient's suitability for receiving CD123×CD3 binding molecule therapy in combination with an antagonist of the PD-1/PD-L1 axis for a hematologic malignancy. The determination of PD-L1 expression may be accomplished by assessing the presence of PD-L1-encoding mRNA, or by assessing the presence of PD-L1 in a cellular lysate or extract. Alternatively, the determination of PD-L1 expression may be accomplished by assessing the presence of PD-L1 molecules arrayed on the cell-surface of PD-L1-expressing cells (e.g., PBMCs). Increased expression (e.g., at least 10% of blast cells express, at least 15% of blast cells express, at least 20% of blast cells express, at least 25% of blast cells express, at least 30% of blast cells express, or at least 40% of blast cells express) PD-L1 as so determined is indicative of the patient's suitability for receiving CD123×CD3 binding molecule therapy in combination with an antagonist of the PD-1/PD-L1 axis for a hematologic malignancy (see e.g., WO 2017/214092).

In a further embodiment, CD8+ T-lymphocytes are monitored for increase in the proportion of CD8+ T-lymphocytes in the tumor microenvironment during and/or following the administration of the CD123×CD3 bispecific molecule.

In a further embodiment, the CD123×CD3 binding molecule therapy of the present invention may additionally comprise the administration of an anti-human PD-L1 binding molecule, such as an anti-human PD-L1 antibody, or a diabody having a human PD-L1 binding domain. Anti-human PD-L1 binding molecules that may be used in accordance with this embodiment include atezolizumab, avelumab, and durvalumab (see, e.g., U.S. Pat. Nos. 9,873,740; 8,779,108). The amino acid sequence of the complete heavy and Light Chains of atezolizumab (WHO Drug Information, 2015, Recommended INN: List 74, 29(3):387), durvalumab (WHO Drug Information, 2015, Recommended INN: List 74, 29(3):393-394) and avelumab (WHO Drug Information, 2016, Recommended INN: List 74, 30(1):100-101) are known in the art.

In an alternative further embodiment, the CD123×CD3 binding molecule therapy of the present invention may additionally comprise the administration of an anti-human PD-1 binding molecule, such as an anti-human PD-1 antibody, or a diabody having a human PD-1 binding domain. Anti-human PD-1 binding molecules that may be used in accordance with this embodiment include: nivolumab (also known as 5C4, BMS-936558, ONO-4538, MDX-1106, and marketed as OPDIVO® by Bristol-Myers Squibb), pembrolizumab (formerly known as lambrolizumab, also known as MK-3475, SCH-900475, and marketed as KEYTRUDA® by Merck), EH12.2H7 (commercially available from BioLegend), pidilizumab (CAS Reg. No.: 1036730-42-3 also known as CT-011, CureTech), hPD-1 mAb 7(1.2) IgG4 (P), and DART-I (disclosed in WO 2017/019846), (also see, e.g., U.S. Pat. Nos. 5,952,136; 7,488,802; 7,521,051; 8,008,449; 8,088,905; 8,354,509; 8,552,154; 8,779,105; 8,900,587; 9,084,776; PCT Patent Publications WO 2004/056875; WO 2006/121168; WO 2008/156712; WO 2012/135408; WO 2012/145493; WO 2013/014668; WO 2014/179664; WO 2014/194302; WO 2015/112800; WO 2017/019846, and WO 2017/214092).

### 1. Exemplary “Target” Genes

IFN gamma stimulates gene expression of more than 200 genes, which include primary response genes such as the IRFs, Fc-gamma receptor (FCGR), GBPs (guanylate-binding proteins), the major histocompatibility complex (MHC) class I and class II molecules, proteins involved in antigen presentation, antiviral proteins such as PKR, and OAS proteins, etc. (Boehm, U. et al. (1997) “Cellular Responses To Interferon-γ,” Annu. Rev. Immunol. 15:749-795; Schroder, K. et al. (2003) “Interferon-Gamma: An Overview Of Signals, Mechanisms And Functions,” J. Leukoc. Biol. 75(2):163-189).

Table 1 discloses exemplary target genes and a representative, non-limiting GenBank® Accession Number for each gene (see, Der, S. D. et al. (1988) “Identification Of Genes Differentially Regulated By Interferon α, β, or γ Using Oligonucleotide Arrays,” Proc. Natl. Acad. Sci. (U.S.A.) 95:15623-15628; Schneider, W. M. et al. (2014) “Interferon-Stimulated Genes: A Complex Web of Host Defenses,” Annu. Rev. Immunol. 32:513-545), and those disclosed in Schroder, K. et al. (2003) (“Interferon-Gamma: An Overview Of Signals, Mechanisms And Functions,” J. Leukoc. Biol. 75(2):163-189), which documents are herein incorporated by reference.

TABLE 1

| Gene Description | GenBank Accession No. |
|---|---|
| 52-kD SS-A/Ro autoantigen | M62800 |
| 9-27 | J04164 |
| Acid finger protein | U09825 |
| ADAR | X79448 |
| AF-1p | Z29064 |
| Alpha-1 type XVI collagen | M92642 |
| Arginosuccinate synthetase | AY034076.1 |
| β-2 microglobulin | J00105 |
| B7.2 | L25259.1 |
| BAK | X84213 |
| Bcl-2 binding component 3 (bbc3) | U82987 |
| BST-2 | D28137 |
| BTG1 | X61123 |
| C2 | X04481 |
| C4 | V00502 |
| Cathepsin B | M14221 |
| Cathepsin D | M11233 |
| Cathepsin H | X16832 |
| Cathepsin L | M86553 |
| c-myc | V00568 |
| Complement component C1r | J04080 |
| DAP var 1 | NM001291963 |
| DAP var 2 | NM004394 |
| dsRNA adenosine deaminase | U10439 |
| DMA | X76775 |
| DMB | X76776 |
| DSS1 | U41515 |
| eIF-2B α subunit | X95648 |
| Fas/Apo-1 | X83492 |
| Fra-1 | X16707 |
| FcγR | X14356 |
| GBP-1 | NM002053 |
| GBP-2 | M55542 |
| gp67phox (CD33) | M23197 |
| gp91phox | M66390 |
| GTPase (rhoC) | L25081 |
| GTP-cyclohydroxylase I | BC025415 |
| HLA class-I (HLA-A26) heavy chain | D32129 |
| Hou | U32849 |
| Human 17q21 clone LF113 | U18009 |
| Human HLA-B null allele | D49824 |
| ICAM-1 | M24283 |
| IFI 16 | M63838 |
| IFN-induced 17/15-kDa protein | M13755 |
| IFN-induced nuclear phosphoprotein | L22342 |
| IFN-γ-inducible gene, 1-5111 | L07633 |
| IFP35 | U72882 |
| IL12 | AF180562 |
| IL15RA | U31628 |
| iNOS | AF049656 |
| Interleukin BSF-2 | X04602 |
| IP-30 | J03909 |
| IRF-1 | X14454 |
| ISG-54K | M14660 |
| L-3-hydroxy acyl-CoA dehydrogenase | X96752 |
| LIM protein MLP | U49837 |
| LMP2 | X66401 |
| LMP7 | Z14982 |
| Lupus p70 (Ku) autoantigen | J04611 |
| MAC-1 (Complement receptor CR3) | X98172 |
| MACH-1 | X98172 |
| Mad1 | AF123318 |
| MCP-1/JE (CCL2) | X14768 |
| MHC Class I | M20022 |
| MHC Class I | X58536 |
| MHC Class I | M21533 |
| MHC Class II α1 | NP002113 |
| MHC Class II α2 | NP064440 |
| MHC Class II β1 | M33907 |
| MHC Class II β2 | NM001300790 |
| Mitochondrial 3-ketoacyl-CoA thiolase | D16481 |
| Mitochondrial SSB | M94556 |
| Mixed lineage kinase 2 | X90846 |
| NF-IL6-beta | M83667 |
| NRAMP1 | D50402 |
| p21 | L25610 |
| p27 | AB003177 |
| p48/ISGF3γ | M876503 |
| p202 | NP001135451 |
| PA28α | AF078829 |
| PA28β | AF079558 |
| Phospholipid scramblase | AF008445 |
| PKR | M35663 |
| PLOD2 | U84573 |
| PMA-responsive gene (APR) | D90070 |
| PML-1 | M79462 |
| PML-2 | M79463 |
| Pol II elongation factor-like protein | Z47087 |
| Poly (ADP-ribose) polymerase | J03473 |
| PPP3CA | L14778 |
| PQ-rich protein | Z50194 |
| PR264 | X75755 |
| PRAME | U65011 |
| Protein phosphatase 5 | X89416 |
| Proton-ATPase-like protein | D89052 |
| RANTES | M21121 |
| RAP46/Bag-1 | Z35491 |
| RbAp48 | X74262 |
| RIG-G | U52513 |
| RING4 | X57522 |
| RTEF-1 | U63824 |
| SAP-1 | M85164 |
| Scaffold protein Pbp1 | U83463 |
| Somatic cytochrome c | M22877 |
| Splicing factor SF3a120 | X85237 |
| SRP9 | U20998 |
| STAT1 (84 kDa) | M97936 |
| STAT1 (91 kDa) | M97935 |
| TAP-1 | L21205 |
| TAP-2 | D42066 |
| Tapasin | AF009510 |
| Tis11d | U07802 |
| TNF-α | NM_000591 |
| Unproductively rearranged IgM | M21388 |
| VCAM-1 | X53051 |
| VEGF-C/VRP | U43142 |

As provided herein, a highly preferred gene expression signature for determining whether a patient had an immune-enriched and IFN gamma-dominant tumor microenvironment is referred to herein as an “Interferon (IFN) Gamma Signaling Signature.” The genes of the IFN Gamma Signaling Signature are: CXCL9, CXCL10, CXCL11, and STAT1 (Table 6). The IFN Gamma Signaling Signature may further comprise IFNG (see, e.g., representative NCBI sequence accession number:NM_000619.2). Increased expression of the IFN Gamma Signaling Signature is particularly correlated to a patient's suitability for CD123×CD3 bispecific binding molecule therapy.

Additional suitable target genes can be added. Such additional target genes may be readily identified as being downstream regulated genes of IFN gamma using the INTERFEROME Database (Samarajiwal, S. A. et al. (2009) “INTERFEROME: The Database Of Interferon Regulated Genes,” Nucleic Acids Research 37: D852-D857). Particularly, preferred additional genes are PDCD1 (also referred to herein by the common name PDL1), PDCD1LG2 (also referred to here by the common name PDL2), IL10, CTLA4 (Table 13), and/or one or more of the genes those present in the following gene signatures: “Interferon (IFN) Downstream Signature” (the genes of which are listed in Table 12B); the “Myeloid Inflammation Signature” (the genes of which are listed in Table 12C); the “Inflammatory Chemokines Signature” (the genes of which are listed in Table 12D) the “MAGES Signature” (the genes of which are listed in Table 12E) and/or the “Immunoproteasome Signature” (the genes of which are listed in Table 12F), provided in the Examples below.

In particular, the expression of multiple genes and signatures can be evaluated in the aggregate as a “module” to evaluate a patient's suitability for CD123×CD3 bispecific binding molecule therapy. One particularly preferred module, which may be used to determine whether a patient exhibits an Immune-infiltrated (immune-enriched) IFN-dominant tumor microenvironment is referred to herein as an “IFN Dominant Module.” The target genes associated with the IFN Dominant Module include: PDL1, PDL2, IL10, CTLA4, and the genes present in each of the following gene expression signatures: the IFN Gamma Signaling Signature, the Interferon Downstream Signature, the Myeloid Inflammation Signature, the Inflammatory Chemokines Signature, the MAGES Signature, and the Immunoproteasome Signature (Table 10).

The IFN Dominant Module is said to be “increased” if the module score is at least about 24, at least about 25, at least about 26, at least about 27, at least about 28, at least about 29, at least about 30, at least about 31, at least about 32, at least about 33, or at least about 35.

The IFN Dominant Module score of a patient is also said to be “increased” if it is greater than the first quartile of IFN Dominant Module scores (i.e., greater than the bottom 25%), greater than the second quartile of IFN Dominant Module scores (i.e., greater than the lower 50%), greater than the third quartile of IFN Dominant Module scores (i.e., greater than the lower 75%), greater than 85%, greater than 90%, or greater than 95% of the IFN Dominant Module scores calculated from the expression levels of such target genes in a population of individuals who are suffering from a hematologic malignancy.

The IFN Dominant Module score of a patient is also said to be “increased” if it is greater than the first quartile of IFN Dominant Module scores (i.e., greater than the bottom 25%), greater than the second quartile of IFN Dominant Module scores (i.e., greater than the lower 50%), greater than the third quartile of IFN Dominant Module scores (i.e., greater than the lower 75%), greater than 85%, greater than 90%, or greater than 95% of the IFN Dominant Module scores calculated from the expression levels of such target genes in a population of individuals who did not successfully respond to a treatment for a hematologic malignancy (e.g., a population of individuals who did not successfully respond to a treatment for a hematologic malignancy CD123×CD3 bispecific molecule).

The IFN Dominant Module score of a patient is also said to be “increased” if it is within at least the first quartile of IFN Dominant Module scores (i.e., within the bottom 25%), and more preferably, within at least the second quartile (i.e., between the bottom 25% and 50%), within at least the third quartile (i.e., between the bottom 50% and 75%), greater than 85%, greater than 90%, or greater than 95% of IFN Dominant Module scores calculated from the expression levels of such target genes in a population of individuals who have previously been successfully treated for a hematologic malignancy using the methods and compositions of the present invention (e.g., a population of individuals who successfully responded to a treatment for a hematologic malignancy using a CD123×CD3 bispecific molecule).

The gene signatures associated with the IFN Dominant Module (CD274, PDCD1LG2, IL10, CTLA4, IFN Gamma Signaling Signature, Interferon Downstream Signature, Myeloid Inflammation Signature, Inflammatory Chemokines Signature, MAGES Signature, and Immunoproteasome Signature) can be individually evaluated to evaluate a patient's suitability for CD123×CD3 bispecific binding molecule therapy.

As further provided herein, another set of highly preferred target genes that may be used to determine whether a patient exhibits a gene expression signature associated with suppressed adaptive immune response within tumors (also referred to herein as a “Tumor Inflammation Signature, or simply as “TIS”) includes the genes: CCL5, CD27, CD274, CD276, CD8A, CMKLR1, CXCL9, CXCR6, HLA-DQA1, HLA-DRB1, HLA-E, IDO1, LAG3, NKG7, PDCD1LG2, PSMB10, STAT1, and/or TIGIT (Table 12A). Increased expression of the Tumor Inflammation Signature is particularly correlated to a patient's suitability for CD123×CD3 bispecific binding molecule therapy.

### 2. Exemplary “Reference” Genes

Housekeeping genes that are constitutively expressed at the same level in normal and malignant cells comprise a preferred class of reference genes. Housekeeping genes include genes involved in general gene expression (such as genes encoding transcription factors, repressors, RNA splicing factors, translation factors, tRNA synthetases, RNA binding proteins, ribosomal proteins, mitochondrial ribosomal proteins, RNA polymerases, protein processing factors, heat shock proteins, histones, cell cycle regulators, apoptosis, oncogenes, DNA repair/replication, etc.), metabolism (such as genes encoding enzymes of: carbohydrate metabolism, the citric acid cycle, lipid metabolism, amino acid metabolism, NADH dehydrogenases, cytochrome C oxidase, ATPases, lysosomal enzymes, proteasome proteins, ribonucleases, thioreductases, etc.), cellular structural integrity (such as genes encoding cytoskeletal proteins, proteins involved in organelle synthesis, mitochondrial proteins, etc.), and cell-surface proteins (such as genes encoding cellular adhesion proteins, ion channels and transporters, receptors, HLA/immunoglobulin/cell recognition proteins, etc.), kinases/signaling proteins (such as growth factors, tissue necrosis factor, casein kinase, etc.). Reference genes that are suitable for this purpose include genes that encode:
sterol regulatory element binding proteins (e.g., ATF1, ATF2, ATF4, ATF6, ATF7, ATF7, BTF3, E2F4, ERH, HMGB1, ILF2, IER2, JUND, TCEB2, etc.);
repressors (e.g., PUF60, etc.);
RNA splicing proteins (e.g., BAT1, HNRPD, HNRPK, PABPN1, SRSF3, etc.);
translation factors (e.g., EIF1, EIF1AD, EIF1B, EIF2A, EIF2AK1, EIF2AK3, EIF2AK4, EIF2AK1, EIF2B2, EIF2B3, EIF2B4, EIF2S2, EIF3A, EIF3B, EIF3D, EIF3G, EIF3I, EIF3H, EIF3J, EIF3K, EIF3L, EIF3M, EIF3S5, EIF3S8, EIF4A1, EIF4A2, EIF4A3, EIF4E2, EIF4G1, EIF4G2, EIF4G3, EIF4H, EIF5, EIF5, EIF5A, EIF5AL1, EIF5B, EIF6, TUFM, etc.);
tRNA synthetases (e.g., AARS, AARS2, AARSD1434, CARS, CARS2, DARS, DARS2, EARS2614, FARS2, FARSA, FARSB, GARS, HARS, HARS2, IARS, IARS2, KARS, LARS2, MARS, MARS2, NARS, NARS2, QARS, RARS, RARS2, SARS, TARS, VARS2, WARS2, YARS, YARS2436, etc.);
RNA binding proteins (e.g., ELAVL1, etc.);
ribosomal proteins (e.g., RPL5, RPL8, RPL9, RPL10A, RPL11, RPL14, RPL25, RPL26L1, RPL27, RPL30, RPL32, RPL34, RPL35, RPL35A, RPL36AL, RPS5, RPS6, RPS6KA3, RPS6KB1, RPS6KB2, RPS13, RPS19BP1, RPS20, RPS23, RPS24, RPS27, RPN1, etc.);
mitochondrial ribosomal proteins (e.g., MRPL9, MRPL1, MRPL10, MRPL11, MRPL12, MRPL13, MRPL14, MRPL15, MRPL16, MRPL17, MRPL18, MRPL19, MRPL2, MRPL20, MRPL21, MRPL22, MRPL23, MRPL24, MRPL27, MRPL28, MRPL3, MRPL30, MRPL32, MRPL33, MRPL35, MRPL36, MRPL37, MRPL38, MRPL4, MRPL40, MRPL41, MRPL42, MRPL43, MRPL44, MRPL45, MRPL46, MRPL47, MRPL48, MRPL49, MRPL50, MRPL51, MRPL52, MRPL53, MRPL54, MRPL55, MRPL9, MRPS10, MRPS11, MRPS12, MRPS14, MRPS15, MRPS16, MRPS17, MRPS18A, MRPS18B, MRPS18C, MRPS2, MRPS21, MRPS22, MRPS23, MRPS24, MRPS25, MRPS26, MRPS27, MRPS28, MRPS30, MRPS31, MRPS33, MRPS34, MRPS35, MRPS5, MRPS6, MRPS7, MRPS9, etc.);
RNA polymerases (e.g., POLR1C, POLR1D, POLR1E, POLR2A, POLR2B, POLR2C, POLR2D, POLR2E, POLR2F, POLR2G, POLR2H, POLR2I, POLR2J, POLR2K, POLR2L, POLR3C, POLR3E, POLR3GL, POLR3K, etc.);
protein processing proteins (e.g., PPID, PPIE, PPIF, PPIG, PPIH, CANX, CAPN1, CAPN7, CAPNS1, NACA, NACA2, PFDN2, PFDN4, PFDN5, PFDN6, SNX2, SNX3, SNX4, SNX5, SNX6, SNX9, SNX12, SNX13, SNX17, SNX18, SNX19, SNX25, SSR1, SSR2, SSR3, SUMO1, SUMO3, etc.);
heat shock proteins (e.g., HSPA4, HSPA5, HSPA8, HSPA9, HSPA14, HSBP1, etc.);
histones (e.g., HIST1H2BC, H1FX, H2AFV, H2AFX, H2AFY, H2AFZ, etc.);
cell cycle proteins (e.g., ARHGAP35, ARHGAP5, ARHGDIA, ARHGEF10L, ARHGEF11, ARHGEF40, ARHGEF7, RAB10, RAB11A, RAB11B, RAB14, RAB18, RAB1A, RAB1B, RAB21, RAB22A, RAB2A, RAB2B380, RAB3GAP1, RAB3GAP2, RAB40C, RAB4A, RAB5A, RAB5B, RAB5C, RAB6A, RAB7A, RAB9A, RABEP1, RABEPK, RABGEF1, RABGGTA, RABGGTB, CENPB, CTBP1, CCNB1IP1, CCNDBP1, CCNG1, CCNH, CCNK402, CCNL1, CCNL2, CCNY, PPP1CA, PPP1CC, PPP1R10, PPP1R11, PPP1R15B, PPP1R37, PPP1R7, PPP1R8, PPP2CA, PPP2CB552, PPP2R1A, PPP2R2A, PPP2R2D, PPP2R3C, PPP2R4, PPP2R5A, PPP2R5B, PPP2R5C, PPP2R5D, PPP2R5E, PPP4C, PPP4R1, PPP4R2, PPP5C, PPP6C, PPP6R2, PPP6R3, RAD1, RAD17, RAD23B, RAD50, RAD51C, IST1, etc.);
apoptosis proteins (e.g., DAD1, DAP3, DAXX, etc.);
oncogene proteins (e.g., ARAF, MAZ, MYC, etc.);
DNA repair/replication proteins (e.g., MCM3AP, XRCC5, XRCC6, etc.);
metabolism proteins (e.g., PRKAG1, PRKAA1, PRKAB1, PRKACA, PRKAG1, PRKAR1A, PRKRIP1, etc.);
carbohydrate metabolism proteins (e.g., ALDOA, B3GALT6, B4GALT3, B4GALT5, B4GALT7, GSK3A, GSK3B, TPI1, PGK1, PGAM5, ENOPH1, LDHA, TALDO1, TSTA3);
citric acid cycle proteins (e.g., SDHA, SDHAF2, SDHB, SDHC, SDHD, etc.);
lipid metabolism proteins (e.g., HADHA, etc.);
amino acid metabolism proteins (e.g., COMT, etc.);
NADH dehydrogenases (e.g., NDUFA2, NDUFA3, NDUFA4, NDUFA5, NDUFA6, NDUFA7, NDUFA8, NDUFA9, NDUFA10, NDUFA11, NDUFA12, NDUFA13, NDUFAF2, NDUFAF3, NDUFAF4, NDUFB2, NDUFB3, NDUFB4, NDUFB5, NDUFB6, NDUFB7, NDUFB10, NDUFB11, NDUFB8, NDUFB9, NDUFC1, NDUFC2, NDUFC2, NDUFS5, NDUFV2, NDUFS2, NDUFS3, NDUFS4, NDUFS5, NDUFS6, NDUFS7, NDUFS8, NDUFV1, NDUFV2, etc.);
cytochrome C oxidases (e.g., COX4I1, COX5B, COX6B1, COX6C, COX7A2, COX7A2L, COX7C, COX8, COX8A, COX11, COX14, COX15, COX16, COX19617, COX20, CYC1, UQCC, UQCR10, UQCR11, UQCRB, UQCRC1, UQCRC2, UQCRHL591, UQCRQ, ATPase, ATP2C1, ATP5A1, ATP5B, ATP5C1, ATP5D, ATP5F1, ATP5G2, ATP5G3, ATP5H, ATP5J, ATP5J2, ATP5J2, ATP5L, ATP5O, ATP5S, ATP5SL, ATP6AP1, ATP6V0A2, ATP6V0B, ATP6V0C, ATP6V0D1, ATP6V0E1, ATP6V1C1, ATP6V1D, ATP6V1E1, ATP6V1F, ATP6V1G1, ATP6V1H, ATPAF2, ATPIF1, etc.);
lysosomal proteins (e.g., CTSD, CSTB, LAMP1, LAMP2, M6PR, etc.);
proteasomal proteins (e.g., PSMA1, PSMA2, PSMA3, PSMA4, PSMA5, PSMA6, PSMA7, PSMB1, PSMB2, PSMB3, PSMB4, PSMB5, PSMB6, PSMB7, PSMC2, PSMC3, PSMC4, PSMC5, PSMC6, PSMD1, PSMD10, PSMD11, PSMD12, PSMD13, PSMD14, PSMD2, PSMD3, PSMD4, PSMD5, PSMD6, PSMD7, PSMD8, PSMD9, PSME2, PSME3, PSMF1, PSMG2, PSMG3, PSMG4591, UBA1, UBA2, UBA3, UBA5, UBA52, UBAC2, UBALD1, UBAP1, UBAP2L, UBB, UBC, UBE2A, UBE2B, UBE2D2, UBE2D3, UBE2D4, UBE2E1, UBE2E2, UBE2E3, UBE2F, UBE2G2, UBE2H, UBE2I, UBE2J1, UBE2J2, UBE2K, UBE2L3, UBE2M, UBE2N, UBE2NL989, UBE2Q1, UBE2R2, UBE2V1, UBE2V2, UBE2W, UBE2Z, UBE3A, UBE3B, UBE3C, UBE4A, UBE4B, USP10, USP14, USP16, USP19, USP22, USP25, USP27X073, USP33, USP38, USP39, USP4, USP47, USP5, USP7, USP8, USP9X590, etc.);
ribonucleases (e.g., RNH, etc.);
thioreductases (e.g., TXN2, TXNDC11, TXNDC12, TXNDC15, TXNDC17, TXNDC9, TXNL1, TXNL4A, TXNL4B, TXNRD1, Cytoskeletal, ANXA6, ANXA7, ARPC1A, ARPC2, ARPC5L, CAPZA2, CAPZB, RHOB, RHOT1, RHOT2, TUBB, WDR1, etc.);
proteins involved in organelle synthesis (e.g., BLOC1S1, BLOC1S2, BLOC1S3, BLOC1S4, BLOC1S6, AP1G1, AP1M1, AP2A1, AP2A2, AP2M1, AP2S1, AP3B1, AP3D1, AP3M1, AP3S1, AP3S2, AP4B1, AP5M1, ANXA6, ANXA7, AP1B1, CLTA, CLTB, CLTC, etc.);
mitochondrial proteins (e.g., MTX2, etc.);
cell surface proteins (e.g., AP2S1, CD81, GPAA1, LGALS9, MGAT2, MGAT4B, VAMP3, etc.);
cell adhesion proteins (e.g., CTNNA1, CTNNB1, CTNNBIP1, CTNNBL1, CTNND1458, etc.);
ion channels and transporter proteins (e.g., ABCB10, ABCB7, ABCD3, ABCE1, ABCF1, ABCF2, ABCF3, CALM1, MFSD11, MFSD12, MFSD3, MFSD5, SLC15A4, SLC20A1, SLC25A11, SLC25A26, SLC25A28, SLC25A3, SLC25A32, SLC25A38, SLC25A39, SLC25A44, SLC25A46, SLC25A5, SLC27A4, SLC30A1, SLC30A5, SLC30A9, SLC35A2, SLC35A4, SLC35B1, SLC35B2, SLC35C2, SLC35E1, SLC35E3, SLC35F5, SLC38A2, SLC39A1, SLC39A3, SLC39A7, SLC41A3, SLC46A3, SLC48A1, Receptors, ACVR1, ACVR1B, CD23, etc.);
HLA/immunoglobulin/cell recognition proteins (e.g., BAT1, BSG, MIF, TAPBP, etc.);
kinases/signaling proteins (e.g., ADRBK1, AGPAT1, ARF1, ARF3, ARF4, ARF5, ARL2, CSF1, CSK, DCT, EFNA3, FKBP1A, GDI1, GNAS1, GNAI2, HAX1, ILK, MAPKAPK2, MAP2K2, MAP3K11, PITPNM, RAC1, RAP1B, RAGA, STK19, STK24, STK25, YWHAB, YWHAH, YWHAQ, YWHAZ, etc.);
growth factors (e.g., AIF1, HDGF, HGS, LTBP4, VEGFB, ZFP36L1, tissue necrosis factor, CD40, casein kinase, CSNK1E, CSNK2B, etc.); and
miscellaneous proteins (e.g., ALAS1, ARHGEF2, ARMET, AES, BECN1, BUD31, CKB, CPNE1, ENSA, FTH1, GDI2, GUK1, HPRT, IFITM1, JTB, MMPL2, NME2, NONO, P4HB, PRDX1, PTMA, RPA2, SULT1A3, SYNGR2, TTC1, C11Orf13, C14orf2, C21orf33, SPAG7, SRM, TEGT, DAZAP2, MEAL etc.).

Preferred housekeeping genes include those listed in Table 2. Table 2 also provides a representative, non-limiting NCBI Accession Number for each gene. Any combination or sub-combination of such genes (and/or splice variants of the same) may be employed.

TABLE 2

| Official | NCBI Accession | Gene | |
|---|---|---|---|
| Symbol | No. | ID | Full Name |
| ABCF1 | NM_001090.2 | 23 | Homo sapiens ATP-binding cassette, |
| | | | sub-family F (GCN20), member 1 |
| | | | (ABCF1), transcript variant 2, mRNA |
| ACTB | NM_001101.2 | 60 | Homo sapiens actin, beta (ACTB), |
| | | | mRNA |
| ALAS1 | NM_000688.4 | 211 | Homo sapiens aminolevulinate, delta-, |
| | | | synthase 1 (ALAS1), transcript variant |
| | | | 1, mRNA |
| B2M | NM_004048.2 | 567 | Homo sapiens beta-2-microglobulin |
| | | | (B2M), mRNA |
| CLTC | NM_004859.2 | 1213 | Homo sapiens clathrin, heavy |
| | | | polypeptide (Hc) (CLTC), mRNA |
| EEF1G | NM_001404.4 | 1937 | Homo sapiens eukaryotic translation |
| | | | elongation factor 1 gamma (EEF1G), |
| | | | mRNA |
| G6PD | NM_000402.2 | 2539 | Homo sapiens glucose-6-phosphate |
| | | | dehydrogenase (G6PD), nuclear gene |
| | | | encoding mitochondrial protein, mRNA |
| GAPDH | NM_002046.3 | 2597 | Homo sapiens glyceraldehyde-3- |
| | | | phosphate dehydrogenase (GAPDH), |
| | | | mRNA |
| GUSB | NM_000181.1 | 2990 | Homo sapiens glucuronidase, beta |
| | | | (GUSB), mRNA |
| HPRT1 | NM_000194.1 | 3251 | Homo sapiens hypoxanthine |
| | | | phosphoribosyltransferase 1 (Lesch- |
| | | | Nyhan syndrome) (HPRT1), mRNA |
| LDHA | NM_005566.1 | 3939 | Homo sapiens lactate dehydrogenase A |
| | | | (LDHA), mRNA |
| NRDE2 | NM_017970.3 | 55051 | Necessary For RNA Interference, |
| | | | Domain Containing |
| OAZ1 | NM_004152.3 | 4946 | Homo sapiens ornithine decarboxylase |
| | | | antizyme 1 (OAZ1), transcript variant |
| | | | 1, mRNA |
| PGK1 | NM_000291.2 | 5230 | Homo sapiens phosphoglycerate kinase |
| | | | 1 (PGK1), mRNA |
| POLR1B | NM_019014.3 | 84172 | Homo sapiens polymerase (RNA) I |
| | | | polypeptide B, 128 kDa (POLR1B), |
| | | | mRNA |
| POLR2A | NM_000937.2 | 5430 | Homo sapiens polymerase (RNA) II |
| | | | (DNA directed) polypeptide A, 220 kDa |
| | | | (POLR2A), mRNA |
| PPIA | NM_021130.4 | 5478 | Homo sapiens peptidylprolyl isomerase |
| | | | A (PPIA), transcript variant 1, mRNA |
| RPL19 | NM_000981.3 | 6143 | Homo sapiens ribosomal protein L19 |
| | | | (RPL19), mRNA |
| RPLP0 | NM_001002.3 | 6175 | Homo sapiens ribosomal protein, large, |
| | | | P0 (RPLP0), transcript variant 1, |
| | | | mRNA |
| SDHA | NM_004168.1 | 6389 | Homo sapiens succinate dehydrogenase |
| | | | complex, subunit A, flavoprotein (Fp) |
| | | | (SDHA), nuclear gene encoding |
| | | | mitochondrial protein, mRNA |
| STK11IP | NM_052902.3 | 114790 | Serine/Threonine Kinase 11 Interacting |
| | | | Protein |
| TBC1D10B | NM_015527.3 | 26000 | TBC1 domain family, member 10B |
| TBP | NM_003194.3 | 6908 | Homo sapiens TATA-box binding |
| | | | protein (TBP), transcript variant 1, |
| | | | mRNA |
| TBP | NM_001172085.1 | 6908 | Homo sapiens TATA-box binding |
| | | | protein (TBP), transcript variant 2, |
| | | | mRNA |
| TUBB | NM_178014.2 | 203068 | Homo sapiens tubulin, beta |
| | | | (TUBB), mRNA |
| UBB | NM_018955.3 | 7314 | Ubiquitin B |

The following reference genes are particularly preferred ABCF1, G6PD, NRDE2, OAZ1, POLR2A, SDHA, STK11IP, TBC1D10B, TBP, and UBB).

### 3. Exemplary Methods for Evaluating Expression of Target and Reference Genes

In order to reveal the level of expression of the target gene(s) relative to the baseline or reference gene(s), the amount of mRNA in a cellular sample corresponding to each assessed target gene is determined and normalized to the expression of mRNA corresponding to the baseline or reference gene(s). Any suitable method may be employed to accomplish such an analysis. A preferred method employs the nCOUNTER® Analysis System (NanoString Technologies, Inc.). In the nCOUNTER® Analysis System, RNA of a sample is incubated in the presence of sets of gene-specific Reporter Probes and Capture Probes under conditions sufficient to permit the sample RNA to hybridize to the probes. Each Reporter Probe carries a fluorescent barcode and each Capture Probe contains a biotin moiety capable of immobilizing the hybridized complex to a solid support for data collection. After hybridization, excess probe is removed, and the support is scanned by an automated fluorescence microscope. Barcodes are counted for each target molecule. Data analysis is preferably conducted using nSolver® 4.0 Analysis Software (NanoString Technologies, Inc.). The data presented in Example 1 was obtained using PanCancer IO 360™ Gene Expression Panel kits (NanoString Technologies, Inc.) which contain a set of probes for 770 different genes (750 genes cover the key pathways at the interface of the tumor, tumor microenvironment, and immune response, and 20 internal reference genes that may be used for data normalization (Table 5). Gene signature scores are calculated as follows:
Raw data counts for each gene are normalized to the geometric mean of the selected housekeeping (HK) genes (e.g., ABCF1, NRDE2, G6PD, OAZ1, POLR2A, SDHA, STK11IP, TBC1D10B, TBP, UBB) for each sample.
HK normalized data is then normalized to IO 360 panel standards, preferably to those run on the same cartridges as the test samples.
Each normalized gene count is then log transformed.
Once normalized and log transformed, each gene is multiplied by a weight value.
Each of these weighted counts is summed to generate a single score. An adjustment factor, that is a constant is added to the final calculated score (e.g. +7). The adjustment factor may be derived from the lowest observed score (from TCGA and/or cell line analysis), in order for the score range to be above 0.

In Table 3, the genes are categorized as follows: Column 1: Gene Name; Column 2: Internal Reference Gene; Column 3: Cell Type (B: B cells; CD8: CD8 T cells; Cyto: Cytotoxic cells; CD45: CD45-expressing cells; CD56d: NK CD56^{dim} cells; DC: dendritic cells; exhausted CD8 cells; M: macrophages; MC: Mast cells; N: Neutrophils; NK: NK cells; T: T cells; Th1: Th1 cells; Treg: Treg cells); Column 4: Release of Cancer Antigens; Column 5: Cancer Antigen Presentation; Column 6: T Cell Priming and Activation; Column 7: Immune Cell Localization to Tumors; Column 8: Stromal Factors; Column 9: Recognition of Cancer Cells by T-cells; Column 10: Killing of Cancer Cells; Column 11: Myeloid Cell Activity; Column 12: NK Cell Activity; Column 13: Cell Cycle and Proliferation; Column 14: Tumor Intrinsic Factors; Column 15: Immunometabolism; Column 16: Common Signaling Pathway. Genes associated with additional pathways and cell types which make up particular gene expression signatures and methods for calculating gene expression signature scores are provided in the Examples below.

TABLE 3

| | Column Number | | | | | | | | | | | | | | | |
|---|---|---|---|---|---|---|---|---|---|---|---|---|---|---|---|---|
| # | 1 | 2 | 3 | 4 | 5 | 6 | 7 | 8 | 9 | 10 | 11 | 12 | 13 | 14 | 15 | 16 |
| 1 | A2M | − | | − | − | − | + | − | − | − | + | − | − | − | − | − |
| 2 | ABCF1 | + | | − | − | − | − | − | − | − | − | − | − | − | − | − |
| 3 | ACVR1C | − | | − | − | − | − | − | − | − | − | − | − | − | − | + |
| 4 | ADAM12 | − | | − | − | − | − | + | − | − | − | − | − | − | − | − |
| 5 | ADGRE1 | − | | − | − | − | + | − | − | − | − | − | − | − | − | − |
| 6 | ADM | − | | − | − | − | − | − | − | − | − | − | − | − | + | − |
| 7 | ADORA2A | − | | − | − | + | − | − | + | − | − | − | − | − | − | − |
| 8 | AKT1 | − | | − | − | − | − | − | − | − | − | − | − | − | + | + |
| 9 | ALDOA | − | | − | − | − | − | + | − | − | − | − | − | − | + | − |
| 10 | ALDOC | − | | − | − | − | − | − | − | − | − | − | − | − | + | − |
| 11 | ANGPT1 | − | | − | − | − | − | + | − | − | + | − | − | − | − | + |
| 12 | ANGPT2 | − | | − | − | − | + | + | − | − | + | − | − | − | − | − |
| 13 | ANGPTL4 | − | | − | − | − | − | − | − | − | − | − | − | − | + | − |
| 14 | ANLN | − | | − | − | − | − | − | − | − | − | − | + | − | − | − |
| 15 | APC | − | | − | − | − | − | − | − | + | − | − | − | − | − | + |
| 16 | APH1B | − | | − | − | − | − | − | − | − | − | − | − | − | − | + |
| 17 | API5 | − | | − | − | − | − | − | − | − | − | − | − | + | − | − |
| 18 | APLNR | − | | − | − | − | − | − | − | − | − | − | − | − | − | + |
| 19 | APOE | − | | − | − | − | + | − | − | − | + | − | − | + | − | − |
| 20 | APOL6 | − | | − | − | − | − | − | − | − | − | − | − | + | − | − |
| 21 | AQP9 | − | | − | − | − | − | − | − | − | − | − | − | − | + | − |
| 22 | AREG | − | | − | − | − | − | − | − | − | + | − | − | − | − | − |
| 23 | ARG1 | − | | − | − | − | − | − | − | − | + | − | − | + | − | − |
| 24 | ARG2 | − | | − | + | − | − | + | − | − | + | − | − | + | − | − |
| 25 | ARID1A | − | | + | − | − | − | − | − | − | − | − | − | + | − | − |
| 26 | ARNT2 | − | | − | − | − | − | − | − | − | − | − | − | − | + | − |
| 27 | ATF3 | − | | − | + | − | + | − | − | − | + | − | − | − | − | − |
| 28 | ATM | − | | + | − | − | − | − | − | − | − | − | + | + | − | − |
| 29 | AXIN1 | − | | − | − | − | − | − | − | − | − | − | − | − | − | + |
| 30 | AXL | − | | − | − | − | + | + | − | − | + | − | − | − | − | − |
| 31 | B2M | − | | − | + | − | − | − | − | − | − | − | − | − | − | − |
| 32 | BAD | − | | − | − | − | − | − | − | + | − | − | − | + | − | + |
| 33 | BAMBI | − | | − | − | − | − | − | − | − | − | − | − | − | − | + |
| 34 | BATF3 | − | | − | + | + | + | − | − | − | + | − | − | − | + | + |
| 35 | BAX | − | | − | − | − | − | − | − | + | − | − | + | + | − | + |
| 36 | BBC3 | − | | − | − | − | − | − | − | + | − | − | − | − | − | + |
| 37 | BBS1 | − | | − | − | − | − | + | − | − | − | − | − | − | − | − |
| 38 | BCAT1 | − | | − | − | − | − | − | − | − | − | − | − | − | + | − |
| 39 | BCL2 | − | | − | − | − | − | − | − | + | − | − | + | + | − | + |
| 40 | BCL2L1 | − | | − | − | − | − | − | − | + | − | − | − | + | − | + |
| 41 | BCL6B | − | | − | − | − | − | − | − | − | − | − | − | − | − | + |
| 42 | BID | − | | − | − | − | − | − | − | − | − | − | + | + | − | − |
| 43 | BIRC3 | − | | + | − | − | − | − | − | − | − | − | − | + | − | − |
| 44 | BIRC5 | − | | − | − | − | − | − | − | − | − | − | − | + | − | − |
| 45 | BLK | − | B | − | + | − | + | − | − | − | − | − | − | − | − | − |
| 46 | BLM | − | | + | − | − | − | − | − | − | − | − | − | + | − | − |
| 47 | BMP2 | − | | − | − | − | − | − | − | − | − | − | − | − | − | + |
| 48 | BNIP3 | − | | + | − | − | − | − | − | − | − | − | − | + | − | − |
| 49 | BNIP3L | − | | − | − | − | − | − | − | − | − | − | − | + | − | − |
| 50 | BRCA1 | − | | + | − | − | − | − | − | − | − | − | − | + | − | − |
| 51 | BRCA2 | − | | + | − | − | − | − | − | − | − | − | − | + | − | − |
| 52 | BRD3 | − | | + | − | − | − | − | − | − | − | − | − | + | − | − |
| 53 | BRD4 | − | | + | − | − | − | − | + | − | − | − | + | + | − | − |
| 54 | BRIP1 | − | | + | − | − | − | − | − | − | − | − | − | + | − | − |
| 55 | BTLA | − | | − | − | + | − | − | + | − | − | − | − | − | − | − |
| 56 | C1QA | − | | − | − | − | − | − | − | − | + | − | − | − | − | − |
| 57 | C1QB | − | | − | − | − | − | − | − | − | + | − | − | − | − | − |
| 58 | C2 | − | | − | − | − | − | − | − | − | + | − | − | − | − | − |
| 59 | C5 | − | | − | − | − | − | − | − | − | + | − | − | − | − | − |
| 60 | C5AR1 | − | | − | − | − | − | − | − | − | + | − | − | − | − | − |
| 61 | C7 | − | | − | − | − | − | − | − | − | + | − | − | − | − | − |
| 62 | CASP1 | − | | − | − | − | − | − | − | + | − | − | − | − | − | − |
| 63 | CASP3 | − | | − | − | − | − | − | − | + | − | − | − | − | − | − |
| 64 | CASP8 | − | | − | − | − | − | − | − | + | − | − | − | − | − | − |
| 65 | CASP9 | − | | − | − | − | − | − | − | + | − | − | − | − | − | − |
| 66 | CBLC | − | | − | − | − | − | − | − | + | − | − | − | − | − | − |
| 67 | CCL13 | − | DC | − | − | − | + | + | − | − | + | − | − | − | − | − |
| 68 | CCL14 | − | | − | − | − | + | − | − | − | − | − | − | − | − | − |
| 69 | CCL18 | − | | − | − | − | + | − | − | − | − | − | − | − | − | − |
| 70 | CCL19 | − | | − | − | − | + | − | − | − | + | − | − | + | − | − |
| 71 | CCL2 | − | | − | − | + | + | + | + | + | + | − | − | − | − | − |
| 72 | CCL20 | − | | − | − | − | + | − | − | − | + | − | − | − | − | − |
| 73 | CCL21 | − | | − | − | − | + | − | − | − | + | − | − | + | − | − |
| 74 | CCL22 | − | | − | + | + | + | − | − | − | + | − | − | − | + | + |
| 75 | CCL3/L1 | − | | − | + | + | + | − | + | + | + | − | − | − | − | − |
| 76 | CCL4 | − | | − | + | + | + | − | + | + | + | − | − | − | − | − |
| 77 | CCL5 | − | | − | + | + | + | − | + | + | + | − | − | − | − | − |
| 78 | CCL7 | − | | − | − | − | + | − | − | − | − | − | − | − | − | − |
| 79 | CCL8 | − | | − | − | − | + | + | − | − | + | − | − | − | − | − |
| 80 | CCNA1 | − | | + | − | − | − | − | + | − | − | − | + | − | − | − |
| 81 | CCNB1 | − | | − | − | − | − | − | − | − | − | − | + | − | − | − |
| 82 | CCND1 | − | | + | − | − | − | − | + | − | − | − | + | − | − | − |
| 83 | CCND2 | − | | − | − | − | − | + | − | − | − | − | − | − | − | − |
| 84 | CCND3 | − | | − | − | − | − | − | − | − | − | − | + | − | − | − |
| 85 | CCNE1 | − | | − | − | − | − | + | − | − | − | − | − | − | − | − |
| 86 | CCNO | − | | + | − | − | − | − | − | − | − | − | − | + | − | − |
| 87 | CCR2 | − | | − | − | + | + | − | + | + | + | − | − | − | − | − |
| 88 | CCR4 | − | | − | − | − | + | − | − | − | − | − | − | − | − | − |
| 89 | CCR5 | − | | − | + | + | + | − | + | + | + | − | − | − | − | − |
| 90 | CD14 | − | | − | + | − | + | + | − | − | + | − | − | − | − | − |
| 91 | CD163 | − | M | − | − | − | + | − | − | − | + | − | − | + | − | − |
| 92 | CD19 | − | B | − | + | − | + | − | − | − | − | − | − | − | − | − |
| 93 | CD1C | − | | − | + | + | + | − | + | − | + | − | − | − | + | − |
| 94 | CD2 | − | | − | − | + | + | − | + | − | + | − | − | − | + | − |
| 95 | CD209 | − | DC | − | − | − | + | − | − | − | − | − | − | − | − | − |
| 96 | CD244 | − | eCD8 | − | − | − | + | − | − | − | − | − | − | − | − | − |
| 97 | CD247 | − | | − | − | + | + | − | − | − | + | − | − | + | − | − |
| 98 | CD27 | − | | − | + | + | + | − | + | + | − | − | − | − | − | − |
| 99 | CD274 | − | | − | + | + | + | − | + | + | + | − | − | − | + | − |
| 100 | CD276 | − | | − | + | − | + | − | + | + | − | − | − | − | − | − |
| 101 | CD28 | − | | − | − | + | − | − | + | − | − | − | − | − | − | − |
| 102 | CD300A | − | | − | − | − | − | − | − | − | − | − | − | − | + | − |
| 103 | CD36 | − | | − | − | − | + | − | − | − | + | − | − | + | − | − |
| 104 | CD38 | − | | − | + | − | + | − | − | − | − | − | − | − | − | − |
| 105 | CD3D | − | T | − | − | + | + | − | + | + | + | − | − | + | − | − |
| 106 | CD3E | − | T | − | + | + | + | − | + | + | + | − | − | − | + | − |
| 107 | CD3G | − | T | − | − | + | + | − | + | + | + | − | − | − | − | − |
| 108 | CD4 | − | | − | + | + | + | − | + | + | − | − | − | − | − | − |
| 109 | CD40 | − | | − | − | + | − | − | + | − | − | − | − | − | − | − |
| 110 | CD40LG | − | | − | − | + | + | − | − | − | − | − | − | − | − | − |
| 111 | CD44 | − | | − | − | + | − | + | − | − | − | − | − | + | − | − |
| 112 | CD45RA | − | | − | − | − | + | − | − | − | − | − | − | + | − | − |
| 113 | CD45RB | − | | − | − | − | + | − | − | − | − | − | − | + | − | − |
| 114 | CD45RO | − | | − | − | − | + | − | − | − | − | − | − | − | − | − |
| 115 | CD47 | − | | − | − | − | + | − | − | + | + | − | − | + | − | − |
| 116 | CD48 | − | | − | − | + | − | − | + | − | − | − | − | − | − | − |
| 117 | CD5 | − | | − | − | + | + | − | + | − | + | − | − | + | + | − |
| 118 | CD58 | − | | − | + | − | − | − | − | − | − | − | − | − | − | − |
| 119 | CD6 | − | T | − | − | − | + | − | − | − | − | − | − | − | − | − |
| 120 | CD68 | − | M | − | + | + | + | − | + | + | − | − | − | − | − | − |
| 121 | CD69 | − | | − | − | + | + | − | + | + | + | − | − | − | − | − |
| 122 | CD7 | − | | − | − | − | + | − | − | − | − | − | − | − | − | − |
| 123 | CD70 | − | | − | − | + | + | − | + | + | + | − | + | − | + | − |
| 124 | CD74 | − | | − | + | − | − | + | − | − | + | − | − | − | − | − |
| 125 | CD79A | − | | − | − | − | + | − | − | − | − | − | − | − | − | − |
| 126 | CD79B | − | | − | + | − | + | − | − | − | − | − | − | − | − | − |
| 127 | CD80 | − | | − | + | + | + | − | + | − | − | − | − | − | − | − |
| 128 | CD84 | − | M | − | − | − | + | − | − | − | − | − | − | − | − | − |
| 129 | CD86 | − | | − | + | + | + | − | + | − | − | − | − | − | − | − |
| 130 | CD8A | − | CD8 | − | + | + | + | − | + | + | + | − | − | − | − | − |
| 131 | CD8B | − | CD8 | − | − | − | + | − | − | − | − | − | − | − | − | − |
| 132 | CD96 | − | | − | − | − | − | − | − | − | − | + | − | − | − | − |
| 133 | CDC20 | − | | − | − | − | − | − | − | − | − | − | + | − | − | − |
| 134 | CDC25C | − | | − | − | − | − | − | − | − | − | − | + | − | − | − |
| 135 | CDH1 | − | | − | − | − | + | + | − | − | + | − | − | + | − | − |
| 136 | CDH11 | − | | − | − | − | − | + | − | − | − | − | − | + | − | − |
| 137 | CDH2 | − | | − | − | − | − | − | − | − | − | − | − | + | − | − |
| 138 | CDH5 | − | | − | − | − | + | − | − | − | − | − | − | + | − | − |
| 139 | CDK2 | − | | − | − | − | − | − | − | − | − | − | + | − | − | − |
| 140 | CDK6 | − | | − | − | − | − | − | − | − | − | − | + | − | − | − |
| 141 | CDKN1A | − | | − | − | − | − | − | − | − | + | − | + | + | − | − |
| 142 | CDKN1C | − | | − | − | − | − | − | − | − | − | − | + | − | − | − |
| 143 | CDKN2A | − | | − | − | − | − | − | − | − | − | − | + | − | − | − |
| 144 | CDKN2B | − | | − | − | − | − | − | − | − | − | − | + | − | − | + |
| 145 | CEACAM3 | − | N | − | − | − | + | − | − | − | − | − | − | + | − | − |
| 146 | CEBPB | − | | − | − | − | − | − | − | − | + | − | − | − | − | − |
| 147 | CENPF | − | | − | − | − | − | − | − | − | − | − | + | − | − | − |
| 148 | CEP55 | − | | − | − | − | − | − | − | − | − | − | + | − | − | − |
| 149 | CES3 | − | | − | − | − | − | + | − | − | − | − | − | − | − | − |
| 150 | CHUK | − | | − | − | − | − | − | − | − | − | − | − | − | − | + |
| 151 | CLEC14A | − | | − | − | − | + | − | − | − | − | − | − | − | − | − |
| 152 | CLEC4E | − | | − | − | − | + | − | − | − | + | − | − | − | − | − |
| 153 | CLEC5A | − | | − | − | − | + | − | − | − | + | − | − | − | − | − |
| 154 | CLEC7A | − | | − | − | − | + | − | − | − | + | − | − | − | − | − |
| 155 | CLECL1 | − | | − | − | + | − | − | − | − | − | − | − | − | − | − |
| 156 | CMKLR1 | − | | − | + | − | + | − | − | + | + | − | − | − | − | − |
| 157 | CNTFR | − | | − | − | − | − | − | − | + | − | − | − | − | − | − |
| 158 | COL11A1 | − | | − | − | − | − | + | − | − | + | − | − | − | − | + |
| 159 | COL11A2 | − | | − | − | − | − | + | − | − | − | − | − | − | − | + |
| 160 | COL17A1 | − | | − | − | − | − | + | − | − | + | − | − | − | − | − |
| 161 | COL4A5 | − | | − | − | − | − | + | − | − | − | − | − | − | − | + |
| 162 | COL5A1 | − | | − | − | − | − | + | − | − | − | − | − | + | − | − |
| 163 | COL6A3 | − | | − | − | − | − | + | − | − | − | − | − | + | − | − |
| 164 | COMP | − | | − | − | − | − | − | − | − | − | − | − | − | − | + |
| 165 | CPA3 | − | MC | − | − | − | + | − | − | − | − | − | − | − | − | − |
| 166 | CRABP2 | − | | − | − | − | − | − | − | − | + | − | − | − | − | − |
| 167 | CSF1 | − | | − | − | − | + | − | − | − | + | − | − | − | − | − |
| 168 | CSF1R | − | | − | − | − | + | − | − | − | + | − | − | − | − | + |
| 169 | CSF2 | − | | − | − | − | + | − | − | − | − | − | − | − | − | − |
| 170 | CSF2RB | − | | − | − | − | − | − | − | − | + | − | − | + | − | − |
| 171 | CSF3 | − | | − | − | − | + | − | − | − | − | − | − | − | − | − |
| 172 | CSF3R | − | N | − | − | − | + | − | − | − | + | − | − | − | + | + |
| 173 | CST2 | − | | − | + | − | − | − | − | − | − | − | − | − | − | − |
| 174 | CTAG1B | − | | − | − | − | − | − | + | − | − | − | − | + | − | − |
| 175 | CTLA4 | − | | − | + | + | + | − | + | − | + | − | − | − | + | − |
| 176 | CTNNB1 | − | | − | − | − | − | − | − | + | − | − | − | − | − | + |
| 177 | CTSS | − | | − | + | − | − | + | − | − | + | − | − | − | − | − |
| 178 | CTSW | − | Cyto | − | + | − | + | − | − | − | + | − | − | + | − | − |
| 179 | CX3CL1 | − | | − | − | + | + | − | + | − | + | − | − | − | + | − |
| 180 | CX3CR1 | − | | − | − | − | + | − | − | − | − | − | − | − | − | − |
| 181 | CXCL1 | − | | − | + | − | + | − | − | + | + | − | − | + | + | − |
| 182 | CXCL10 | − | | − | + | + | + | − | + | + | + | − | − | − | − | − |
| 183 | CXCL11 | − | | − | + | + | + | − | + | + | + | − | − | − | − | − |
| 184 | CXCL12 | − | | − | + | − | + | + | − | − | + | − | − | − | − | − |
| 185 | CXCL13 | − | | − | − | + | + | − | + | + | + | − | − | − | − | − |
| 186 | CXCL14 | − | | − | − | − | + | − | − | − | − | − | − | − | − | − |
| 187 | CXCL16 | − | | − | − | − | + | − | − | + | − | − | − | − | − | − |
| 188 | CXCL2 | − | | − | − | − | + | − | − | − | + | − | − | − | − | − |
| 189 | CXCL3 | − | | − | − | − | + | − | − | − | + | − | − | − | − | − |
| 190 | CXCL5 | − | | − | − | − | + | − | − | − | + | − | − | − | − | − |
| 191 | CXCL6 | − | | − | − | − | + | − | − | − | + | − | − | − | − | − |
| 192 | CXCL8 | − | | − | − | − | + | − | − | − | + | − | − | − | − | − |
| 193 | CXCL9 | − | | − | + | + | + | − | + | + | + | − | − | − | − | − |
| 194 | CXCR2 | − | | − | − | − | + | − | − | − | − | − | − | − | − | − |
| 195 | CXCR3 | − | | − | − | + | + | − | + | + | + | − | − | − | − | − |
| 196 | CXCR4 | − | | − | − | − | + | − | − | − | − | − | + | − | − | − |
| 197 | CXCR6 | − | | − | + | − | + | − | − | + | − | − | − | − | − | − |
| 198 | CXorf36 | − | | − | − | − | + | − | − | − | − | − | − | − | − | − |
| 199 | CYBB | − | | − | − | − | + | − | − | − | + | − | − | − | − | − |
| 200 | DAB2 | − | | − | − | − | − | − | − | − | + | − | − | − | − | − |
| 201 | DDB2 | − | | + | − | − | − | − | − | − | − | − | − | + | − | − |
| 202 | DEFB134 | − | | − | − | − | − | − | − | + | − | − | − | − | − | − |
| 203 | DEPTOR | − | | − | − | − | − | − | − | − | − | − | − | − | + | − |
| 204 | DKK1 | − | | − | − | − | − | − | − | − | − | − | − | − | − | + |
| 205 | DLL1 | − | | − | − | − | − | − | − | − | − | − | − | − | − | + |
| 206 | DLL4 | − | | − | − | − | − | + | − | − | + | − | − | − | − | + |
| 207 | DNAJC14 | + | | − | − | − | − | − | − | − | − | − | − | − | − | − |
| 208 | DNMT1 | − | | + | − | − | − | − | − | − | − | − | − | + | − | − |
| 209 | DPP4 | − | | − | − | + | + | − | + | − | + | − | − | + | + | − |
| 210 | DTX3L | − | | + | − | − | − | − | − | − | − | − | − | − | − | − |
| 211 | DTX4 | − | | − | − | − | − | − | − | − | − | − | − | − | − | + |
| 212 | DUSP1 | − | | − | − | − | − | − | − | − | − | − | − | − | − | + |
| 213 | DUSP2 | − | | − | − | − | − | − | − | − | − | − | − | − | − | + |
| 214 | DUSP5 | − | | − | − | − | − | − | − | − | − | − | − | − | − | + |
| 215 | E2F3 | − | | − | − | − | − | + | − | − | − | − | − | − | − | − |
| 216 | EDN1 | − | | − | − | − | − | + | − | − | − | − | − | − | − | − |
| 217 | EGF | − | | − | − | − | − | − | − | − | − | − | − | − | − | + |
| 218 | EGFR | − | | − | − | − | − | − | − | − | − | − | − | − | − | + |
| 219 | EGR1 | − | | − | − | + | + | − | + | − | + | − | − | + | + | − |
| 220 | EIF2AK2 | − | | − | − | − | − | − | − | + | − | − | − | − | − | − |
| 221 | EIF2B4 | − | | − | − | − | − | − | − | − | − | − | − | − | − | + |
| 222 | EIF4EBP1 | − | | − | − | − | − | − | − | − | − | − | − | − | + | − |
| 223 | EIF5AL1 | − | | + | − | − | − | − | − | − | − | − | − | + | − | − |
| 224 | ELOB | − | | − | + | − | − | − | − | − | − | − | − | − | − | − |
| 225 | ENO1 | − | | − | − | − | − | − | − | − | − | − | − | − | + | + |
| 226 | ENTPD1 | − | | − | − | − | − | − | − | − | − | − | − | + | − | − |
| 227 | EOMES | − | eCD8 | − | − | + | + | − | + | + | + | − | − | − | + | − |
| 228 | EPCAM | − | | − | − | − | + | + | − | + | − | − | − | + | − | − |
| 229 | EPM2AIP1 | − | | + | − | − | − | − | − | − | − | − | − | + | − | − |
| 230 | ERBB2 | − | | − | − | − | − | − | − | − | − | − | − | − | − | + |
| 231 | ERCC3 | + | | − | − | − | − | − | − | − | − | − | − | − | − | − |
| 232 | ERO1A | − | | − | − | − | − | − | − | + | − | − | − | − | − | − |
| 233 | ESR1 | − | | − | − | − | − | − | − | − | − | − | − | − | − | + |
| 234 | EXO1 | − | | − | − | − | − | − | − | − | − | − | + | − | − | − |
| 235 | EZH2 | − | | − | − | − | − | + | − | − | − | − | − | − | − | − |
| 236 | F2RL1 | − | | − | − | + | + | + | + | − | + | − | − | − | + | − |
| 237 | FADD | − | | − | − | − | − | − | − | + | − | − | − | + | − | − |
| 238 | FAM124B | − | | + | − | − | − | − | − | − | − | − | − | − | − | − |
| 239 | FAM30A | − | | − | − | − | + | − | − | − | − | − | − | − | − | − |
| 240 | FANCA | − | | + | − | − | − | − | − | − | − | − | − | + | − | − |
| 241 | FAP | − | | − | − | − | + | + | − | − | + | − | − | − | − | − |
| 242 | FAS | − | | − | − | − | + | − | − | − | + | − | − | + | + | − |
| 243 | FASLG | − | | + | − | − | − | − | − | + | − | − | − | + | − | + |
| 244 | FBP1 | − | | − | − | − | − | − | − | − | − | − | − | − | + | − |
| 245 | FCAR | − | N | − | − | − | + | − | − | − | + | − | − | − | − | − |
| 246 | FCGR1A | − | | − | − | − | + | − | − | − | + | − | − | − | − | − |
| 247 | FCGR2A | − | | − | − | − | + | − | − | − | + | − | − | + | − | − |
| 248 | FCGR2B | − | | − | − | − | − | − | − | − | + | − | − | − | − | − |
| 249 | FCGR3A/B | − | | − | − | − | + | − | − | − | + | − | − | − | − | − |
| 250 | FCGRT | − | | − | − | − | − | − | − | − | + | − | − | − | − | − |
| 251 | FCN1 | − | | − | − | − | + | − | − | − | + | − | − | − | − | − |
| 252 | FCRL2 | − | B | − | − | − | + | − | − | − | − | − | − | − | − | − |
| 253 | FGF13 | − | | − | − | − | − | − | − | − | − | − | − | − | − | + |
| 254 | FGF18 | − | | − | − | − | − | + | − | − | − | − | − | − | − | − |
| 255 | FGF9 | − | | − | − | − | − | − | − | − | − | − | − | − | − | + |
| 256 | FGFR1 | − | | − | − | − | − | + | − | − | − | − | − | − | − | − |
| 257 | FLNB | − | | − | − | − | − | − | − | + | − | − | − | − | − | − |
| 258 | FLT1 | − | | − | − | − | + | + | − | − | + | − | − | − | − | − |
| 259 | FOSL1 | − | | − | − | − | − | − | − | − | + | − | − | − | − | + |
| 260 | FOXP3 | − | Treg | − | + | + | + | − | + | + | − | − | − | − | − | − |
| 261 | FPR1 | − | N | − | − | − | + | − | − | − | + | − | − | − | − | − |
| 262 | FPR3 | − | | − | − | − | + | − | − | − | + | − | − | − | − | − |
| 263 | FSTL3 | − | | − | − | − | − | + | − | − | − | − | − | − | − | − |
| 264 | FUT4 | − | | − | − | − | − | − | − | − | − | − | − | − | + | − |
| 265 | FYN | − | | − | − | + | + | − | − | − | + | − | − | + | − | − |
| 266 | FZD8 | − | | − | − | − | − | − | − | − | − | − | − | − | − | + |
| 267 | FZD9 | − | | − | − | − | − | − | − | + | − | − | − | − | − | + |
| 268 | G6PD | + | | − | − | − | − | − | − | − | − | − | − | − | − | − |
| 269 | GAS1 | − | | − | − | − | − | − | − | − | − | − | − | − | − | + |
| 270 | GBP1 | − | | − | − | − | − | − | − | + | − | − | − | − | − | − |
| 271 | GBP2 | − | | − | − | − | − | − | − | + | − | − | − | − | − | − |
| 272 | GBP4 | − | | − | − | − | − | − | − | + | − | − | − | − | − | − |
| 273 | GHR | − | | − | − | − | − | − | − | + | − | − | − | − | − | − |
| 274 | GIMAP4 | − | | − | − | − | − | − | − | − | − | − | − | + | − | − |
| 275 | GIMAP6 | − | | − | − | − | − | − | − | − | − | − | − | + | − | − |
| 276 | GLI1 | − | | − | − | − | − | − | − | − | − | − | − | − | − | + |
| 277 | GLS | − | | − | − | − | − | − | − | − | − | − | − | − | + | + |
| 278 | GLUD1 | − | | − | − | − | − | − | − | − | − | − | − | − | + | − |
| 279 | GLUL | − | | − | − | − | − | − | − | − | − | − | − | − | + | − |
| 280 | GMIP | − | | − | − | − | − | − | − | − | − | − | − | − | − | + |
| 281 | GNG4 | − | | − | − | − | − | − | − | − | − | − | − | − | − | + |
| 282 | GNLY | − | Cyto | − | + | + | + | − | + | + | + | + | − | − | − | − |
| 283 | GOT1 | − | | − | − | − | − | − | − | − | − | − | − | − | + | + |
| 284 | GOT2 | − | | − | − | − | − | − | − | − | − | − | − | − | + | + |
| 285 | GPC4 | − | | − | − | − | − | − | − | − | − | − | − | − | − | + |
| 286 | GPR160 | − | | − | − | − | − | − | − | − | − | − | − | + | − | − |
| 287 | GPSM3 | − | | − | − | − | − | − | − | − | − | − | − | − | − | + |
| 288 | GUSB | + | | − | − | − | − | − | − | − | − | − | − | − | − | − |
| 289 | GZMA | − | Cyto | − | + | + | + | − | + | + | + | + | − | + | − | − |
| 290 | GZMB | − | Cyto | − | + | + | + | − | + | + | + | + | − | + | − | − |
| 291 | GZMH | − | Cyto | − | − | + | + | − | + | + | + | + | − | + | − | − |
| 292 | GZMK | − | | − | − | + | + | − | + | + | + | + | − | + | − | − |
| 293 | GZMM | − | | − | − | + | + | − | + | + | + | + | − | − | − | − |
| 294 | H2AFX | − | | + | − | − | − | − | − | − | − | − | − | + | − | − |
| 295 | HAVCR2 | − | | − | − | + | − | − | + | − | − | − | − | − | − | − |
| 296 | HCK | − | | − | − | − | + | − | − | − | + | − | − | − | − | − |
| 297 | HDAC11 | − | | + | − | − | − | − | − | − | − | − | − | + | − | − |
| 298 | HDAC3 | − | | + | − | − | − | − | − | − | − | − | − | + | − | − |
| 299 | HDAC4 | − | | + | − | − | − | − | − | − | − | − | − | + | − | − |
| 300 | HDAC5 | − | | + | − | − | − | − | − | − | − | − | − | + | − | − |
| 301 | HDC | − | MC | − | − | − | + | − | − | − | − | − | − | − | − | − |
| 302 | HELLS | − | | + | − | − | − | − | − | − | − | − | − | + | − | − |
| 303 | HERC6 | − | | − | − | − | − | − | − | − | − | − | − | − | − | − |
| 304 | HES1 | − | | − | − | − | − | − | − | − | − | − | − | − | − | + |
| 305 | HEY1 | − | | − | − | − | − | + | − | − | − | − | − | − | − | − |
| 306 | HIF1A | − | | − | − | − | − | + | − | − | − | − | − | − | + | + |
| 307 | HK1 | − | | − | − | − | − | − | − | − | − | − | − | − | + | − |
| 308 | HK2 | − | | − | − | − | − | − | − | − | − | − | − | − | + | + |
| 309 | HLA-A | − | | − | + | − | − | − | − | − | − | − | − | − | − | − |
| 310 | HLA-B | − | | − | + | − | − | − | − | − | − | − | − | − | − | − |
| 311 | HLA-C | − | | − | + | − | − | − | − | − | − | − | − | − | − | − |
| 312 | HLA-DMA | − | | − | + | + | + | + | + | + | + | − | − | − | − | − |
| 313 | HLA-DMB | − | | − | + | + | + | − | + | + | − | − | − | − | − | − |
| 314 | HLA-DOA | − | | − | + | + | + | − | + | + | − | − | − | − | − | − |
| 315 | HLA-DOB | − | | − | + | + | + | − | + | + | − | − | − | − | − | − |
| 316 | HLA-DPA1 | − | | − | + | − | − | + | − | − | + | − | − | − | − | − |
| 317 | HLA-DPB1 | − | | − | + | − | − | + | − | − | + | − | − | − | − | − |
| 318 | HLA-DQA1 | − | | − | + | − | + | − | − | + | − | − | − | − | − | − |
| 319 | HLA-DQA2 | − | | − | + | + | + | − | + | + | − | − | − | − | − | − |
| 320 | HLA-DQB1 | − | | − | + | + | + | + | + | + | + | − | − | − | − | − |
| 321 | HLA-DRA | − | | − | + | + | + | − | + | + | − | − | − | − | − | − |
| 322 | HLA-DRB1 | − | | − | + | − | + | − | − | + | − | − | − | − | − | − |
| 323 | HLA-DRB5 | − | | − | + | + | + | + | + | + | + | − | − | − | − | − |
| 324 | HLA-E | − | | − | + | − | + | − | − | + | − | − | − | − | − | − |
| 325 | HLA-F | − | | − | + | − | − | − | − | − | − | − | − | − | − | − |
| 326 | HMGA1 | − | | + | − | − | − | − | − | − | − | − | − | + | − | − |
| 327 | HMGB1 | − | | − | − | − | − | − | − | − | + | − | − | − | − | − |
| 328 | HNF1A | − | | − | − | − | − | − | − | + | − | − | − | + | − | + |
| 329 | HRAS | − | | − | − | − | − | − | − | − | − | − | − | + | + | + |
| 330 | HSD11B1 | − | DC | − | − | − | + | − | − | − | − | − | − | − | − | − |
| 331 | ICAM1 | − | | − | + | + | + | + | + | + | + | − | − | + | + | + |
| 332 | ICAM2 | − | | − | + | + | + | + | + | + | − | − | − | + | − | − |
| 333 | ICAM3 | − | | − | + | + | + | + | + | + | − | − | − | + | − | − |
| 334 | ICAM5 | − | | − | − | − | + | − | − | − | − | − | − | − | − | − |
| 335 | ICOS | − | | − | − | + | + | − | + | + | + | − | − | − | − | − |
| 336 | ICOSLG | − | | − | + | + | + | − | − | − | − | − | − | − | − | − |
| 337 | ID4 | − | | − | − | − | − | − | − | − | − | − | − | − | − | + |
| 338 | IDO1 | − | | − | + | + | + | − | + | + | + | − | − | + | + | − |
| 339 | IER3 | − | | − | − | − | − | − | − | − | + | − | − | − | − | − |
| 340 | IFI16 | − | | − | − | − | + | − | − | + | − | + | − | − | − | − |
| 341 | IFI27 | − | | − | − | − | + | − | − | + | + | − | − | − | + | − |
| 342 | IFI35 | − | | − | − | − | + | − | − | + | − | + | − | − | − | − |
| 343 | IFI6 | − | | − | − | − | − | − | − | + | + | − | − | − | + | − |
| 344 | IFIH1 | − | | − | − | − | + | − | − | + | − | + | − | − | − | − |
| 345 | IFIT1 | − | | − | − | − | + | − | − | + | − | − | − | − | − | − |
| 346 | IFIT2 | − | | − | − | − | + | − | − | + | − | + | − | − | − | − |
| 347 | IFIT3 | − | | − | − | − | − | − | − | + | + | − | − | − | + | − |
| 348 | IFITM1 | − | | − | − | − | + | − | − | + | + | − | − | + | − | − |
| 349 | IFITM2 | − | | − | − | − | + | − | − | + | − | + | − | − | − | − |
| 350 | IFNA1 | − | | − | − | − | + | − | − | − | + | − | − | − | − | − |
| 351 | IFNAR1 | − | | − | − | − | + | − | − | − | + | − | − | − | − | − |
| 352 | IFNG | − | | − | + | + | + | − | + | + | + | − | − | − | − | − |
| 353 | IFNGR1 | − | | − | + | + | + | − | + | + | + | − | − | − | − | − |
| 354 | IFNGR2 | − | | − | + | + | + | − | + | + | + | − | − | − | − | − |
| 355 | IGF2R | − | | − | − | − | − | − | − | + | − | − | − | − | − | − |
| 356 | IHH | − | | − | − | + | + | − | + | + | + | − | − | − | − | + |
| 357 | IKBKB | − | | − | − | − | − | − | − | − | − | − | − | − | − | + |
| 358 | IKBKG | − | | − | − | − | − | − | − | − | − | − | − | − | − | + |
| 359 | IL10 | − | | + | − | − | + | + | + | − | + | − | + | − | − | + |
| 360 | IL10RA | − | | − | − | − | + | − | − | − | + | − | − | + | − | − |
| 361 | IL11 | − | | − | + | − | + | − | − | + | + | − | − | − | − | − |
| 362 | IL11RA | − | | − | − | − | − | − | − | + | − | − | − | − | − | − |
| 363 | IL12RB2 | − | | − | − | + | + | − | + | + | + | − | − | − | + | − |
| 364 | IL15 | − | | − | − | + | + | − | + | + | + | − | − | − | − | − |
| 365 | IL16 | − | | − | − | − | + | − | − | − | + | − | − | − | − | − |
| 366 | IL17A | − | | − | − | + | + | − | + | + | + | − | − | − | − | − |
| 367 | IL18 | − | | − | − | + | + | − | − | − | + | − | − | − | − | − |
| 368 | IL18R1 | − | | − | − | + | + | − | + | − | + | − | − | − | + | − |
| 369 | ILIA | − | | − | − | − | + | − | − | + | + | − | − | − | + | + |
| 370 | IL1B | − | | − | − | − | + | − | − | + | + | − | − | − | + | + |
| 371 | IL1R2 | − | | − | − | − | + | − | − | − | + | − | − | − | − | − |
| 372 | IL1RN | − | | − | − | − | + | − | − | − | + | − | − | − | − | + |
| 373 | IL2 | − | | − | + | + | + | − | − | − | + | − | − | − | + | + |
| 374 | IL21R | − | CD56d | − | − | − | + | − | − | − | − | − | − | − | − | − |
| 375 | IL22RA1 | − | | − | − | − | + | − | − | + | − | − | − | − | − | − |
| 376 | IL24 | − | | − | − | − | − | − | − | − | + | − | − | − | − | − |
| 377 | IL2RA | − | | − | − | + | − | − | − | + | − | − | − | − | − | + |
| 378 | IL2RB | − | | − | − | + | + | − | − | + | − | − | − | − | − | + |
| 379 | IL2RG | − | | − | − | + | + | − | − | − | − | − | − | − | − | − |
| 380 | IL32 | − | | − | − | − | + | − | − | − | + | − | − | − | − | − |
| 381 | IL33 | − | | − | + | + | + | − | − | − | + | − | − | − | + | + |
| 382 | IL34 | − | | − | − | − | + | − | − | − | + | − | − | − | − | − |
| 383 | IL4 | − | | − | + | + | + | − | − | − | + | − | − | − | + | + |
| 384 | IL6 | − | | − | − | − | − | − | − | + | + | − | − | − | + | + |
| 385 | IL6R | − | | − | − | − | + | − | − | − | + | − | − | − | − | − |
| 386 | IL7R | − | | − | − | − | − | − | + | + | − | − | − | − | − | − |
| 387 | INHBA | − | | − | − | − | − | − | − | − | − | − | − | − | − | + |
| 388 | IRF1 | − | | − | + | + | + | − | + | + | + | − | − | − | − | − |
| 389 | IRF2 | − | | − | − | − | − | − | − | + | − | − | − | − | − | − |
| 390 | IRF3 | − | | − | − | − | − | − | − | + | − | − | − | − | − | − |
| 391 | IRF4 | − | | − | − | + | + | − | + | + | + | − | − | + | + | − |
| 392 | IRF5 | − | | − | − | − | + | − | − | − | − | − | − | + | − | − |
| 393 | IRF7 | − | | − | − | − | − | − | − | + | − | − | − | − | − | − |
| 394 | IRF8 | − | | − | + | − | − | − | − | − | − | − | − | − | − | − |
| 395 | IRF9 | − | | − | − | − | − | − | − | + | + | − | − | − | + | − |
| 396 | ISG15 | − | | − | − | − | − | − | − | + | + | − | − | − | + | − |
| 397 | ITGA1 | − | | − | − | + | + | + | + | − | + | − | − | + | + | − |
| 398 | ITGA2 | − | | − | − | − | + | + | − | − | − | − | − | + | − | − |
| 399 | ITGA4 | − | | − | − | − | + | + | − | − | − | − | − | + | − | − |
| 400 | ITGA6 | − | | − | − | − | + | + | − | − | − | − | − | + | − | − |
| 401 | ITGAE | − | | − | − | + | + | + | + | + | + | − | − | + | − | − |
| 402 | ITGAL | − | | − | − | − | + | + | − | − | + | − | − | + | − | − |
| 403 | ITGAM | − | | − | − | − | + | + | − | − | + | − | − | + | − | − |
| 404 | ITGAV | − | | − | − | − | − | + | − | − | − | − | − | − | − | − |
| 405 | ITGAX | − | | − | − | − | + | + | − | − | + | − | − | + | − | − |
| 406 | ITGB2 | − | | − | − | − | + | + | − | − | − | − | − | + | − | − |
| 407 | ITGB3 | − | | − | − | − | + | + | − | − | − | − | − | + | − | − |
| 408 | ITGB8 | − | | − | − | − | − | − | − | − | − | − | − | − | − | + |
| 409 | ITPK1 | − | | − | − | − | − | + | − | − | − | − | − | − | − | − |
| 410 | JAG1 | − | | − | − | − | − | + | − | − | − | − | − | − | − | − |
| 411 | JAG2 | − | | − | − | − | − | − | − | − | − | − | − | − | − | + |
| 412 | JAK1 | − | | − | − | − | − | − | − | + | − | − | − | − | − | − |
| 413 | JAK2 | − | | − | − | − | − | − | − | + | − | − | − | − | − | − |
| 414 | JAK3 | − | | − | − | − | + | − | − | + | − | − | − | − | − | − |
| 415 | KAT2B | − | | − | − | − | − | − | − | − | − | − | − | − | − | + |
| 416 | KDR | − | | − | − | − | − | + | − | − | − | − | − | − | − | − |
| 417 | KIF2C | − | | − | − | − | − | − | − | − | − | − | + | − | − | − |
| 418 | KIR2DL3 | − | CD56d | − | − | − | + | − | − | − | − | + | − | − | − | − |
| 419 | KIR3DL1 | − | CD56d | − | − | − | + | − | − | − | − | + | − | − | − | − |
| 420 | KIR3DL2 | − | CD56d | − | − | − | + | − | − | − | − | + | − | − | − | − |
| 421 | KIT | − | | − | − | − | − | − | − | + | − | − | − | + | − | + |
| 422 | KLRB1 | − | Cyto | − | − | − | + | − | − | − | − | + | − | − | − | − |
| 423 | KLRD1 | − | Cyto | − | − | − | + | − | − | − | − | + | − | − | − | − |
| 424 | KLRK1 | − | Cyto | − | − | − | + | − | − | − | − | + | − | − | − | − |
| 425 | KRAS | − | | − | − | − | − | − | − | − | − | − | − | − | + | + |
| 426 | LAG3 | − | eCD8 | − | + | + | + | − | + | + | + | − | − | − | − | − |
| 427 | LAIR1 | − | | − | − | − | − | − | − | + | − | − | − | − | − | − |
| 428 | LAMA1 | − | | − | − | − | − | − | − | − | − | − | − | − | − | + |
| 429 | LAMB3 | − | | − | − | − | − | + | − | − | + | − | − | − | − | − |
| 430 | LAMC2 | − | | − | − | − | − | − | − | − | − | − | − | − | − | + |
| 431 | LCK | − | | − | − | + | + | − | + | − | + | − | − | + | + | − |
| 432 | LDHA | − | | − | + | + | + | − | − | − | + | − | − | − | + | + |
| 433 | LDHB | − | | − | + | + | + | − | − | − | + | − | − | − | + | + |
| 434 | LGALS9 | − | | − | − | − | − | − | − | − | + | − | − | − | − | − |
| 435 | LIF | − | | − | − | − | − | − | − | + | + | − | − | + | − | − |
| 436 | LILRA1 | − | | − | + | − | − | − | − | − | − | − | − | − | − | − |
| 437 | LILRA3 | − | | − | + | − | − | − | − | − | − | − | − | − | − | − |
| 438 | LILRA5 | − | | − | − | − | + | − | − | − | + | − | − | − | − | − |
| 439 | LILRB2 | − | | − | − | + | + | − | − | − | + | − | − | − | − | − |
| 440 | LILRB4 | − | | − | − | + | − | − | − | − | − | − | − | − | − | − |
| 441 | LOXL2 | − | | − | − | − | + | + | − | − | + | − | − | − | − | − |
| 442 | LRRC32 | − | | − | − | − | + | − | − | − | − | − | − | − | − | − |
| 443 | LIB | − | | − | − | + | − | − | − | + | + | − | + | − | − | − |
| 444 | LTBP1 | − | | − | − | − | − | − | − | − | − | − | − | − | − | + |
| 445 | LY9 | − | | − | − | + | + | − | − | − | − | − | − | − | − | − |
| 446 | LY96 | − | | − | − | − | + | − | − | − | + | − | − | − | − | − |
| 447 | LYZ | − | | − | − | − | + | − | − | − | + | − | − | − | − | − |
| 448 | MAGEA1 | − | | + | − | − | − | − | − | − | − | − | − | − | − | − |
| 449 | MAGEA12 | − | | + | − | − | − | − | − | − | − | − | − | − | − | − |
| 450 | MAGEA3/A6 | − | | + | − | − | − | − | − | − | − | − | − | − | − | − |
| 451 | MAGEA4 | − | | + | − | − | − | − | − | − | − | − | − | − | − | − |
| 452 | MAGEB2 | − | | + | − | − | − | − | − | − | − | − | − | − | − | − |
| 453 | MAGEC1 | − | | + | − | − | − | − | − | − | − | − | − | − | − | − |
| 454 | MAGEC2 | − | | + | − | − | − | − | − | − | − | − | − | − | − | − |
| 455 | MAML2 | − | | − | − | − | − | − | − | − | − | − | − | − | − | + |
| 456 | MAP3K12 | − | | + | − | − | − | − | − | − | − | − | − | + | − | − |
| 457 | MAP3K5 | − | | − | − | − | − | − | − | − | − | − | − | − | − | + |
| 458 | MAP3K7 | − | | − | − | − | − | − | − | − | − | − | − | − | − | + |
| 459 | MAP3K8 | − | | − | − | − | − | − | − | − | − | − | − | − | − | + |
| 460 | MAPK10 | − | | − | − | − | − | − | − | − | − | − | − | − | − | + |
| 461 | MARCO | − | | − | − | − | + | − | − | − | + | − | − | − | − | − |
| 462 | MB21D1 | − | | − | − | + | − | − | − | − | + | − | − | − | − | − |
| 463 | MELK | − | | − | − | − | − | − | − | − | − | − | + | − | − | − |
| 464 | MET | − | | − | − | − | − | − | − | − | − | − | − | − | − | + |
| 465 | MFGE8 | − | | − | − | − | + | − | − | − | + | − | − | + | − | − |
| 466 | MFNG | − | | − | − | − | − | − | − | − | − | − | − | − | − | + |
| 467 | MGMT | − | | + | − | − | − | − | − | − | − | − | − | + | − | − |
| 468 | MICA | − | | − | + | − | − | − | − | − | − | − | − | − | − | − |
| 469 | MICB | − | | − | − | − | + | − | − | − | + | + | − | + | − | − |
| 470 | MKI67 | − | | − | − | − | − | − | − | − | − | − | + | − | − | − |
| 471 | MLANA | − | | − | − | − | − | − | + | − | − | − | − | − | − | − |
| 472 | MLH1 | − | | + | − | − | − | − | − | − | − | − | − | + | − | − |
| 473 | MMP1 | − | | − | − | − | − | + | − | − | + | − | − | − | − | − |
| 474 | MMP7 | − | | − | − | − | − | + | − | − | − | − | − | − | − | + |
| 475 | MMP9 | − | | − | − | − | − | + | − | − | − | − | − | − | − | − |
| 476 | MMRN2 | − | | − | − | − | + | + | − | − | − | − | − | − | − | − |
| 477 | MRC1 | − | | − | − | − | − | − | − | − | + | − | − | − | − | − |
| 478 | MRE11 | − | | + | − | − | − | − | − | − | − | − | − | − | − | − |
| 479 | MRPL19 | + | | − | − | − | − | − | − | − | − | − | − | − | − | − |
| 480 | MS4A1 | − | B | − | + | − | + | − | − | − | − | − | − | − | − | − |
| 481 | MS4A2 | − | MC | − | − | − | + | − | − | − | − | − | − | − | − | − |
| 482 | MS4A4A | − | M | − | − | − | + | − | − | − | − | − | − | − | − | − |
| 483 | MS4A6A | − | | − | − | − | + | − | − | − | − | − | − | + | − | − |
| 484 | MSH2 | − | | + | − | − | − | − | − | − | − | − | − | + | − | − |
| 485 | MSH6 | − | | + | − | − | − | − | − | − | − | − | − | + | − | − |
| 486 | MTOR | − | | − | − | − | − | − | − | − | − | − | − | − | + | − |
| 487 | MX1 | − | | − | − | − | − | − | − | + | + | − | − | − | + | − |
| 488 | MXI1 | − | | − | − | − | − | − | − | − | + | − | − | − | − | − |
| 489 | MYC | − | | − | − | − | − | − | − | − | − | − | − | − | + | + |
| 490 | MYCT1 | − | | − | − | − | + | − | − | − | − | − | − | − | − | − |
| 491 | MYD88 | − | | − | − | + | − | − | − | − | + | − | − | − | − | − |
| 492 | NBN | − | | + | − | − | − | − | − | − | − | − | − | + | − | − |
| 493 | NCAM1 | − | | − | − | − | + | − | − | + | − | + | − | − | − | − |
| 494 | NCR1 | − | NK | − | − | − | + | − | − | − | − | + | − | − | − | − |
| 495 | NDUFA4L2 | − | | − | − | − | − | − | − | − | − | − | − | − | + | − |
| 496 | NECTIN1 | − | | − | − | − | − | + | − | − | − | − | − | − | − | − |
| 497 | NECTIN2 | − | | − | − | + | − | − | + | − | − | − | − | − | − | − |
| 498 | NEIL1 | − | | + | − | − | − | − | − | − | − | − | − | + | − | − |
| 499 | NF1 | − | | − | − | − | − | − | − | − | − | − | − | − | − | + |
| 500 | NFAM1 | − | | − | − | − | + | − | − | − | + | − | − | − | − | − |
| 501 | NFATC2 | − | | − | − | + | + | − | − | − | + | − | − | + | − | − |
| 502 | NFIL3 | − | | − | − | − | − | + | − | − | − | − | − | − | − | − |
| 503 | NFKB1 | − | | − | − | − | − | − | − | − | − | − | − | − | − | + |
| 504 | NFKB2 | − | | − | − | − | − | − | − | − | − | − | − | − | − | + |
| 505 | NFKBIA | − | | − | − | − | − | − | − | − | − | − | − | − | − | + |
| 506 | NFKBIE | − | | − | − | − | − | − | − | − | − | − | − | − | − | + |
| 507 | NGFR | − | | − | − | − | − | − | − | − | − | − | − | − | − | + |
| 508 | NID2 | − | | − | − | − | − | + | − | − | − | − | − | + | − | − |
| 509 | NKG7 | − | Cyto | − | + | − | + | − | − | + | − | + | − | − | − | − |
| 510 | NLRC5 | − | | − | − | − | − | − | − | − | + | − | − | − | − | − |
| 511 | NLRP3 | − | | − | − | − | + | − | − | − | + | − | − | − | − | − |
| 512 | NOD2 | − | | − | − | − | + | − | − | − | + | − | − | − | − | − |
| 513 | NOS2 | − | | − | − | − | − | − | − | − | + | − | − | − | − | + |
| 514 | NOTCH1 | − | | − | − | − | − | − | − | − | − | − | − | − | − | + |
| 515 | NOTCH2 | − | | − | − | − | − | − | − | − | − | − | − | − | − | + |
| 516 | NRAS | − | | − | − | − | − | − | − | − | − | − | − | − | − | + |
| 517 | NRDE2 | + | | − | − | − | − | − | − | − | − | − | − | − | − | − |
| 518 | NT5E | − | | − | − | − | + | − | + | − | + | − | − | + | − | − |
| 519 | OAS1 | − | | − | − | − | − | − | − | + | + | − | − | − | + | − |
| 520 | OAS2 | − | | − | − | − | − | − | − | + | + | − | − | − | + | − |
| 521 | OAS3 | − | | − | − | − | + | − | − | + | + | − | − | − | − | − |
| 522 | OASL | − | | − | − | − | − | − | − | − | + | − | − | − | − | − |
| 523 | OAZ1 | + | | − | − | − | − | − | − | − | − | − | − | − | − | − |
| 524 | OLFML2B | − | | − | − | − | − | + | − | − | − | − | − | − | − | − |
| 525 | OLR1 | − | | − | − | − | − | − | − | − | + | − | − | − | − | − |
| 526 | OTOA | − | | − | − | − | + | − | − | − | − | − | − | − | − | − |
| 527 | P2RY13 | − | | − | − | − | + | − | − | − | + | − | − | − | − | − |
| 528 | P4HA1 | − | | − | − | − | − | + | − | − | − | − | − | − | − | − |
| 529 | P4HA2 | − | | − | − | − | − | + | − | − | − | − | − | − | − | − |
| 530 | PALMD | − | | − | − | − | − | + | − | − | − | − | − | − | − | − |
| 531 | PARP12 | − | | + | − | − | − | − | − | − | − | − | − | − | − | − |
| 532 | PARP4 | − | | + | − | − | − | − | − | − | − | − | − | + | − | − |
| 533 | PARP9 | − | | + | − | − | − | − | − | − | − | − | − | − | − | − |
| 534 | PC | − | | − | − | − | − | − | − | − | − | − | − | − | + | − |
| 535 | PCK2 | − | | − | − | − | − | − | − | − | − | − | − | − | + | − |
| 536 | PDCD1 | − | | − | + | + | + | − | + | + | − | − | − | − | − | − |
| 537 | PDCD1LG2 | − | | − | + | + | + | − | + | + | − | − | − | − | − | − |
| 538 | PDGFA | − | | − | − | − | − | − | − | − | − | − | − | − | − | + |
| 539 | PDGFB | − | | − | − | − | − | + | − | − | − | − | − | − | − | − |
| 540 | PDGFRB | − | | − | − | − | − | + | − | − | − | − | − | − | − | − |
| 541 | PDK1 | − | | − | − | − | − | − | − | − | − | − | − | − | + | − |
| 542 | PDZK1IP1 | − | | − | − | − | − | − | − | − | + | − | − | − | − | − |
| 543 | PECAM1 | − | | − | − | − | + | − | − | − | + | − | − | + | − | − |
| 544 | PF4 | − | | − | − | + | + | − | + | + | + | − | − | − | − | − |
| 545 | PFKFB3 | − | | − | − | − | − | − | − | − | − | − | − | − | + | + |
| 546 | PFKM | − | | − | − | − | − | − | − | − | − | − | − | − | + | + |
| 547 | PGPEP1 | − | | − | − | − | − | + | − | − | − | − | − | − | − | − |
| 548 | PIAS4 | − | | + | − | − | − | − | − | − | − | − | − | − | − | − |
| 549 | PIK3CA | − | | − | − | − | − | + | − | + | − | − | − | − | + | + |
| 550 | PIK3CD | − | | + | − | − | − | − | − | − | − | − | − | + | − | + |
| 551 | PIK3CG | − | | + | − | − | − | − | − | + | − | − | − | + | − | + |
| 552 | PIK3R1 | − | | − | − | − | − | + | − | − | − | − | − | − | + | + |
| 553 | PIK3R2 | − | | − | − | − | − | − | − | − | − | − | − | − | + | + |
| 554 | PIK3R5 | − | | + | − | − | − | − | − | + | − | − | − | + | − | + |
| 555 | PKM | − | | − | − | − | − | − | − | − | − | − | − | − | + | + |
| 556 | PLA1A | − | | − | − | − | − | − | − | − | − | − | − | − | − | + |
| 557 | PLA2G2A | − | | − | − | − | − | − | − | − | − | − | − | − | − | + |
| 558 | PLOD2 | − | | − | − | − | − | + | − | − | − | − | − | − | − | − |
| 559 | PMS2 | − | | + | − | − | − | − | − | − | − | − | − | + | − | − |
| 560 | PNOC | − | B | − | − | − | + | − | − | − | − | − | − | − | − | − |
| 561 | POLD1 | − | | + | − | − | − | − | − | − | − | − | − | + | − | − |
| 562 | POLR2A | + | | − | − | − | − | − | − | − | − | − | − | − | − | − |
| 563 | PPARG | − | | − | − | − | − | − | − | − | − | − | − | − | + | − |
| 564 | PPARGC1B | − | | − | − | − | − | − | − | − | − | − | − | − | + | − |
| 565 | PRF1 | − | Cyto | − | + | + | + | − | + | + | + | + | − | − | − | − |
| 566 | PRKAA2 | − | | − | − | − | − | − | − | − | − | − | − | − | − | + |
| 567 | PRKACB | − | | − | − | − | − | − | − | − | − | − | − | − | − | + |
| 568 | PRKCA | − | | − | − | − | − | − | − | − | − | − | − | − | + | − |
| 569 | PRKX | − | | − | − | − | − | − | − | − | − | − | − | + | − | + |
| 570 | PRLR | − | | − | − | − | − | − | − | + | − | − | − | − | − | − |
| 571 | PROM1 | − | | − | − | + | + | − | + | + | + | − | − | + | − | − |
| 572 | PRR5 | − | | − | − | − | − | − | − | − | − | − | − | − | + | − |
| 573 | PSMB10 | − | | − | + | − | + | − | − | + | − | − | − | − | − | − |
| 574 | PSMB5 | − | | − | + | − | + | − | − | + | − | − | − | − | − | − |
| 575 | PSMB8 | − | | − | + | − | − | − | − | − | − | − | − | − | − | − |
| 576 | PSMB9 | − | | − | + | + | + | − | + | + | − | − | − | − | − | − |
| 577 | PSMC4 | + | | − | − | − | − | − | − | − | − | − | − | − | − | − |
| 578 | PTCD2 | − | | − | − | − | − | − | − | − | − | − | − | − | + | − |
| 579 | PTEN | − | | − | − | − | − | − | − | + | − | − | − | − | + | + |
| 580 | PTGER4 | − | eCD8 | − | − | − | + | − | − | − | − | − | − | − | − | − |
| 581 | PTGS2 | − | | − | − | + | + | − | − | − | + | − | − | − | − | − |
| 582 | PTPN11 | − | | − | − | − | − | − | − | − | − | − | − | − | − | + |
| 583 | PTPRC | − | CD45 | − | − | − | + | − | − | − | − | − | − | + | − | − |
| 584 | PUM1 | + | | − | − | − | − | − | − | − | − | − | − | − | − | − |
| 585 | PVR | − | | − | − | − | − | − | + | − | − | + | − | − | − | − |
| 586 | PVRIG | − | | − | − | + | − | − | + | − | − | − | − | − | − | − |
| 587 | RAD50 | − | | + | − | − | − | − | − | − | − | − | − | + | − | − |
| 588 | RAD51 | − | | + | − | − | − | − | − | − | − | − | − | + | − | − |
| 589 | RAD51C | − | | + | − | − | − | − | − | − | − | − | − | + | − | − |
| 590 | RASAL1 | − | | − | − | − | − | − | − | − | − | − | − | − | − | + |
| 591 | RASGRF1 | − | | − | − | − | − | − | − | − | − | − | − | − | − | + |
| 592 | RB1 | − | | − | − | − | − | − | − | − | − | − | + | − | − | − |
| 593 | RBL2 | − | | − | − | − | − | − | − | − | − | − | − | − | − | + |
| 594 | RELA | − | | − | − | − | − | − | − | − | − | − | − | − | − | + |
| 595 | RELB | − | | − | − | − | − | − | − | − | − | − | − | − | − | + |
| 596 | RELN | − | | − | − | − | − | − | − | − | − | − | − | − | − | + |
| 597 | REN | − | | − | − | + | + | − | + | + | + | − | − | − | − | − |
| 598 | RICTOR | − | | − | − | − | − | − | − | − | − | − | − | − | + | − |
| 599 | RIPK1 | − | | − | − | − | − | − | − | − | − | − | − | − | − | + |
| 600 | RIPK2 | − | | − | − | − | − | − | − | − | − | − | − | − | − | + |
| 601 | RIPK3 | − | | − | − | − | − | − | − | − | − | − | − | − | − | + |
| 602 | RNLS | − | | + | − | − | − | − | − | − | − | − | − | + | − | − |
| 603 | ROBO4 | − | | − | − | − | + | − | − | − | − | − | − | − | − | − |
| 604 | ROCK1 | − | | − | − | − | − | − | − | − | − | − | − | − | − | + |
| 605 | ROR2 | − | | − | − | − | + | + | − | − | + | − | − | − | − | − |
| 606 | RORC | − | | − | − | − | + | − | − | + | − | − | − | + | − | − |
| 607 | RPL23 | − | | − | − | − | − | − | − | − | − | − | − | − | + | − |
| 608 | RPL7A | − | | − | − | − | − | + | − | − | − | − | − | − | − | − |
| 609 | RPS6KB1 | − | | − | − | − | − | + | − | − | − | − | − | − | + | + |
| 610 | RPTOR | − | | − | − | − | − | − | − | − | − | − | − | − | + | − |
| 611 | RRM2 | − | | − | − | − | − | − | − | − | − | − | + | − | − | − |
| 612 | RSAD2 | − | | − | − | + | − | − | − | + | − | − | − | − | − | − |
| 613 | RUNX3 | − | | − | − | − | + | − | − | − | + | − | − | + | − | − |
| 614 | S100A12 | − | N | − | − | − | + | − | − | − | + | − | − | − | − | − |
| 615 | S100A8 | − | | − | − | − | + | − | − | − | + | − | − | − | − | − |
| 616 | S100A9 | − | | − | − | − | + | − | − | − | + | − | − | − | − | − |
| 617 | SAMD9 | − | | − | − | − | − | − | − | − | − | − | − | + | − | − |
| 618 | SAMSN1 | − | | − | − | − | − | − | − | − | − | − | − | + | − | − |
| 619 | SBNO2 | − | | − | − | − | − | − | − | − | + | − | − | − | − | − |
| 620 | SDHA | + | | − | − | − | − | − | − | − | − | − | − | − | − | − |
| 621 | SELE | − | | − | − | − | + | − | − | − | + | − | − | + | − | − |
| 622 | SELL | − | | − | − | − | + | − | − | + | − | + | − | − | − | − |
| 623 | SELP | − | | − | − | − | + | − | − | + | − | + | − | − | − | − |
| 624 | SERPINA1 | − | | − | − | − | + | − | − | − | + | − | − | − | − | − |
| 625 | SERPINB5 | − | | − | − | − | − | + | − | − | − | − | − | − | − | − |
| 626 | SERPINH1 | − | | − | − | − | − | + | − | − | − | − | − | − | − | − |
| 627 | SF3A1 | + | | − | − | − | − | − | − | − | − | − | − | − | − | − |
| 628 | SFRP1 | − | | − | − | − | − | − | − | − | − | − | − | − | − | + |
| 629 | SFRP4 | − | | − | − | − | − | − | − | − | − | − | − | − | − | + |
| 630 | SFXN1 | − | | + | − | − | − | − | − | − | − | − | − | + | − | − |
| 631 | SGK1 | − | | − | − | − | − | − | − | − | − | − | − | − | + | − |
| 632 | SH2D1A | − | T | − | − | − | + | − | − | − | − | − | − | − | − | − |
| 633 | SHC2 | − | | − | − | − | − | − | − | − | − | − | − | − | − | + |
| 634 | SIGLEC1 | − | | − | − | − | + | − | − | − | + | − | − | + | − | − |
| 635 | SIGLEC5 | − | N | − | − | − | + | − | − | − | − | − | − | − | − | − |
| 636 | SIGLEC8 | − | | − | − | − | − | − | − | − | + | − | − | − | − | − |
| 637 | SIRPA | − | | − | − | − | + | − | − | + | + | − | − | − | − | − |
| 638 | SIRPB2 | − | | − | − | − | + | − | − | − | + | − | − | − | − | − |
| 639 | SLAMF7 | − | | − | − | − | − | − | − | − | − | + | − | − | − | − |
| 640 | SLC11A1 | − | | − | − | + | + | − | + | − | + | − | − | − | + | − |
| 641 | SLC16A1 | − | | − | − | − | − | − | − | − | − | − | − | − | + | + |
| 642 | SLC1A5 | − | | − | − | − | − | − | − | − | − | − | − | − | + | − |
| 643 | SLC2A1 | − | | − | − | − | − | − | − | − | − | − | − | − | + | − |
| 644 | SLC7A5 | − | | − | − | − | − | − | − | − | − | − | − | − | + | − |
| 645 | SMAD5 | − | | − | − | − | − | − | − | − | − | − | − | − | − | + |
| 646 | SMAP1 | − | | + | − | − | − | − | − | − | − | − | − | + | − | − |
| 647 | SNAI1 | − | | − | − | − | − | − | − | − | − | − | − | + | − | − |
| 648 | SNCA | − | | − | + | − | − | − | − | − | − | − | − | − | − | − |
| 649 | SOCS1 | − | | − | − | − | − | − | − | − | + | − | − | − | − | + |
| 650 | SOX10 | − | | + | − | − | − | − | − | − | − | − | − | − | − | − |
| 651 | SOX11 | − | | − | − | − | − | − | − | + | − | − | − | − | − | + |
| 652 | SOX2 | − | | − | − | − | − | − | − | + | − | − | − | − | − | + |
| 653 | SPIB | − | B | − | − | − | + | − | − | − | − | − | − | − | − | − |
| 654 | SPP1 | − | | − | − | + | + | + | − | − | − | − | − | − | − | − |
| 655 | SPRY4 | − | | − | − | − | − | − | − | + | − | − | − | − | − | − |
| 656 | SREBF1 | − | | + | − | − | − | − | − | − | − | − | − | + | + | − |
| 657 | SRP54 | − | | − | − | − | − | − | − | − | − | − | − | − | + | − |
| 658 | STAT1 | − | | − | + | + | + | − | + | + | + | − | − | − | − | − |
| 659 | STAT2 | − | | − | − | − | − | − | − | + | + | − | − | − | + | − |
| 660 | STAT3 | − | | − | − | − | − | − | − | − | + | − | − | − | − | + |
| 661 | STAT4 | − | | − | − | + | + | − | + | + | + | − | − | − | − | − |
| 662 | STC1 | − | | − | − | − | − | + | − | − | − | − | − | − | − | − |
| 663 | STK11IP | + | | − | − | − | − | − | − | − | − | − | − | − | − | − |
| 664 | SYK | − | | − | − | − | − | − | − | − | + | − | − | − | − | − |
| 665 | TAF3 | − | | − | − | − | − | − | − | − | − | − | − | − | + | − |
| 666 | TAP1 | − | | − | + | − | − | − | − | − | − | − | − | − | − | − |
| 667 | TAP2 | − | | − | + | − | − | − | − | − | − | − | − | − | − | − |
| 668 | TAPBP | − | | − | + | − | − | − | − | − | − | − | − | − | − | − |
| 669 | TAPBPL | − | | − | + | − | − | − | − | − | − | − | − | − | − | − |
| 670 | TBC1D10B | + | | − | − | − | − | − | − | − | − | − | − | − | − | − |
| 671 | TBP | + | | − | − | − | − | − | − | − | − | − | − | − | − | − |
| 672 | TBX21 | − | Th1 | − | − | + | + | − | + | + | + | − | − | − | − | − |
| 673 | TBXAS1 | − | | − | − | − | − | − | − | − | − | − | − | − | + | − |
| 674 | TCF3 | − | | − | − | − | − | − | − | − | − | − | − | + | − | + |
| 675 | TCL1A | − | B | − | − | − | + | − | − | − | − | − | − | − | − | − |
| 676 | TDO2 | − | | − | − | − | − | − | − | − | − | − | − | + | − | − |
| 677 | TFRC | + | | − | − | − | − | − | − | − | − | − | − | − | − | − |
| 678 | TGFB1 | − | | − | − | − | − | − | + | − | + | − | − | + | − | + |
| 679 | TGFB2 | − | | − | + | + | + | − | − | − | + | − | + | − | + | + |
| 680 | TGFB3 | − | | − | − | − | − | − | − | − | − | − | − | − | − | + |
| 681 | TGFBR1 | − | | − | − | − | − | − | + | − | + | − | − | − | − | + |
| 682 | TGFBR2 | − | | − | − | − | − | − | + | − | + | − | − | − | − | + |
| 683 | THBD | − | | − | + | − | + | − | − | − | − | − | − | − | − | − |
| 684 | THBS1 | − | | − | + | − | − | + | − | − | − | − | + | − | − | + |
| 685 | THY1 | − | | − | − | − | + | + | − | − | − | − | − | + | − | − |
| 686 | TICAM1 | − | | − | − | − | − | − | − | − | + | − | − | − | − | − |
| 687 | TIE1 | − | | − | − | − | − | + | − | − | + | − | − | − | − | − |
| 688 | TIGIT | − | | − | + | + | + | − | + | + | − | + | − | − | − | − |
| 689 | TLK2 | + | | − | − | − | − | − | − | − | − | − | − | − | − | − |
| 690 | TLR1 | − | | − | − | + | + | − | − | − | + | − | − | − | − | − |
| 691 | TLR2 | − | | − | − | + | + | − | − | − | + | − | − | − | − | − |
| 692 | TLR3 | − | | − | − | + | − | − | − | − | + | − | − | − | − | − |
| 693 | TLR4 | − | | − | − | + | + | − | − | − | + | − | − | − | − | − |
| 694 | TLR5 | − | | − | − | + | − | − | − | − | + | − | − | − | − | − |
| 695 | TLR7 | − | | − | − | + | − | − | − | − | + | − | − | − | − | − |
| 696 | TLR8 | − | | − | − | + | + | − | − | − | + | − | − | − | − | − |
| 697 | TLR9 | − | | − | − | + | + | − | − | − | + | − | − | − | − | − |
| 698 | TMEM140 | − | | − | − | − | + | − | − | − | − | − | − | − | − | − |
| 699 | TMEM173 | − | | − | + | + | + | − | − | − | + | − | − | − | + | + |
| 700 | TMUB2 | + | | − | − | − | − | − | − | − | − | − | − | − | − | − |
| 701 | TNF | − | | − | − | + | + | − | − | + | + | − | + | − | − | + |
| 702 | TNFAIP3 | − | | − | − | + | − | − | − | + | + | − | + | − | − | − |
| 703 | TNFAIP6 | − | | − | − | − | − | + | − | − | + | − | − | − | − | − |
| 704 | TNFRSF10B | − | | + | − | + | − | − | − | + | + | − | + | + | − | − |
| 705 | TNFRSF10C | − | | + | − | + | − | − | − | + | + | − | + | + | − | − |
| 706 | TNFRSF10D | − | | + | − | − | − | − | − | − | − | − | − | + | − | − |
| 707 | TNFRSF11A | − | | − | − | + | − | − | − | + | + | − | + | − | − | − |
| 708 | TNFRSF11B | − | | − | − | + | − | − | − | + | + | − | + | − | − | − |
| 709 | TNFRSF14 | − | | − | − | + | − | − | − | + | + | − | + | − | − | − |
| 710 | TNFRSF17 | − | B | − | − | + | + | − | − | + | + | − | + | − | − | − |
| 711 | TNFRSF18 | − | | − | − | + | − | − | − | + | + | − | + | − | − | − |
| 712 | TNFRSF1A | − | | − | − | + | − | − | − | + | + | − | + | − | − | − |
| 713 | TNFRSF1B | − | | − | − | + | − | − | − | + | + | − | + | − | − | − |
| 714 | TNFRSF25 | − | | − | − | + | − | − | + | − | − | − | − | − | − | − |
| 715 | TNFRSF4 | − | | − | − | + | − | − | − | + | + | − | + | − | − | − |
| 716 | TNFRSF8 | − | | − | − | + | − | − | − | + | + | − | + | − | − | − |
| 717 | TNFRSF9 | − | | − | − | + | − | − | − | + | + | − | + | − | − | − |
| 718 | TNFSF10 | − | | + | − | + | − | − | − | + | + | − | + | + | − | − |
| 719 | TNFSF12 | − | | − | − | + | − | − | − | + | + | − | + | − | − | − |
| 720 | TNFSF13 | − | | − | − | + | − | − | − | + | + | − | + | − | − | − |
| 721 | TNFSF13B | − | | − | − | + | − | − | − | + | + | − | + | − | − | − |
| 722 | TNFSF18 | − | | − | − | + | − | − | − | + | + | − | + | − | − | − |
| 723 | TNFSF4 | − | | − | − | + | − | − | − | + | + | − | + | − | − | − |
| 724 | TNFSF8 | − | | − | − | + | − | − | − | + | + | − | + | − | − | − |
| 725 | TNFSF9 | − | | − | − | + | − | − | + | − | − | − | − | − | − | − |
| 726 | TNKS | − | | + | − | − | − | − | − | − | − | − | − | + | − | − |
| 727 | TP53 | − | | + | − | − | − | − | − | − | − | − | − | + | − | − |
| 728 | TPI1 | − | | − | − | − | − | − | − | − | − | − | − | − | + | − |
| 729 | TPM1 | − | | − | − | − | − | + | − | − | − | − | − | − | − | − |
| 730 | TPSAB1/B2 | − | | − | − | − | + | − | − | + | − | − | − | + | − | − |
| 731 | TRAF1 | − | | − | − | − | − | − | − | + | − | − | − | − | − | + |
| 732 | TRAT1 | − | T | − | − | − | + | − | − | − | − | − | − | − | − | − |
| 733 | TREM1 | − | | − | − | − | + | − | − | − | + | − | − | − | − | − |
| 734 | TREM2 | − | | − | − | − | − | − | − | − | + | − | − | − | − | − |
| 735 | TRIM21 | − | | − | − | − | − | − | − | − | + | − | − | − | − | + |
| 736 | TSLP | − | | − | − | + | + | − | + | + | + | − | − | − | − | − |
| 737 | TTC30A | − | | + | − | − | − | − | − | − | − | − | − | + | − | − |
| 738 | TWF1 | − | | − | − | − | + | − | − | − | − | − | − | − | − | − |
| 739 | TWIST1 | − | | − | − | − | − | + | − | − | − | − | − | + | − | − |
| 740 | TWIST2 | − | | − | − | − | + | + | − | − | + | − | − | − | − | − |
| 741 | TYMP | − | | − | − | − | − | + | − | − | − | − | − | − | − | − |
| 742 | TYMS | − | | + | − | − | − | − | − | − | − | − | − | + | − | − |
| 743 | UBA7 | − | | + | − | − | − | − | − | − | − | − | − | − | − | − |
| 744 | UBB | + | | + | − | − | − | − | − | − | − | − | − | + | − | − |
| 745 | UBE2C | − | | − | − | − | − | − | − | − | − | − | + | − | − | − |
| 746 | UBE2T | − | | + | − | − | − | − | − | − | − | − | − | + | − | − |
| 747 | ULBP2 | − | | − | + | − | + | − | − | − | + | − | − | + | − | − |
| 748 | VCAM1 | − | | − | − | − | + | + | − | − | − | − | − | + | − | − |
| 749 | VCAN | − | | − | − | − | − | + | − | − | − | − | − | − | − | − |
| 750 | VEGFA | − | | − | − | + | + | + | + | + | + | − | − | − | − | + |
| 751 | VEGFB | − | | − | − | − | − | + | − | − | − | − | − | − | − | − |
| 752 | VEGFC | − | | − | − | − | + | + | − | − | + | − | − | − | − | − |
| 753 | VHL | − | | − | − | − | − | + | − | − | − | − | − | − | + | − |
| 754 | VSIR | − | | − | − | − | − | − | + | − | − | − | − | − | − | − |
| 755 | VTCN1 | − | | − | − | + | − | − | + | − | − | − | − | − | − | − |
| 756 | WDR76 | − | | + | − | − | − | − | − | − | − | − | − | + | − | − |
| 757 | WNT10A | − | | − | − | − | − | − | − | − | − | − | − | − | − | + |
| 758 | WNT11 | − | | − | − | − | − | − | − | − | − | − | − | − | − | + |
| 759 | WNT2 | − | | − | − | − | − | − | − | − | − | − | − | − | − | + |
| 760 | WNT2B | − | | − | − | − | − | − | − | − | − | − | − | − | − | + |
| 761 | WNT3A | − | | − | − | − | − | − | − | − | − | − | − | − | − | + |
| 762 | WNT4 | − | | − | − | − | − | − | − | − | − | − | − | − | − | + |
| 763 | WNT5A | − | | − | − | − | − | − | − | − | − | − | − | − | − | + |
| 764 | WNT5B | − | | − | − | − | − | − | − | − | − | − | − | − | − | + |
| 765 | WNT7B | − | | − | − | − | − | − | − | + | − | − | − | − | − | + |
| 766 | XCL1/2 | − | | − | − | − | + | − | − | − | − | − | − | − | − | − |
| 767 | ZAP70 | − | | − | − | + | + | − | − | − | − | − | − | − | − | − |
| 768 | ZC3H12A | − | | − | − | − | − | − | − | − | + | − | − | − | − | − |
| 769 | ZEB1 | − | | − | − | − | − | − | − | − | − | − | − | + | − | − |
| 770 | ZEB2 | − | | − | − | − | − | − | − | − | − | − | − | + | − | − |

B. Analysis of “Gene Expression Signatures”

Gene expression analysis of bone marrow (BM) cell samples at baseline stratifies chemotherapy-refractory, HMA-refractory (including secondary AML), and Relapsed patients into 3 cluster groups within an immunological continuum: patients exhibiting an immune-depleted gene expression signature, patients exhibiting an immune-exhausted gene expression signature, and patients exhibiting an immune-enriched gene expression signature.

As described in more detail below, patients with primary-refractory disease (refractory to ≥2 induction attempts, first CR of <6 months, or failure after ≥4 cycles of hypomethylating agents, HMA) exhibit the gene expression signature of an immune-infiltrated tumor microenvironment, as seen by their approximately 33% higher inflammatory chemokine levels (relative to the levels seen in relapse patients (3.27±0.22 vs 2.46±0.07, p=0.026)).

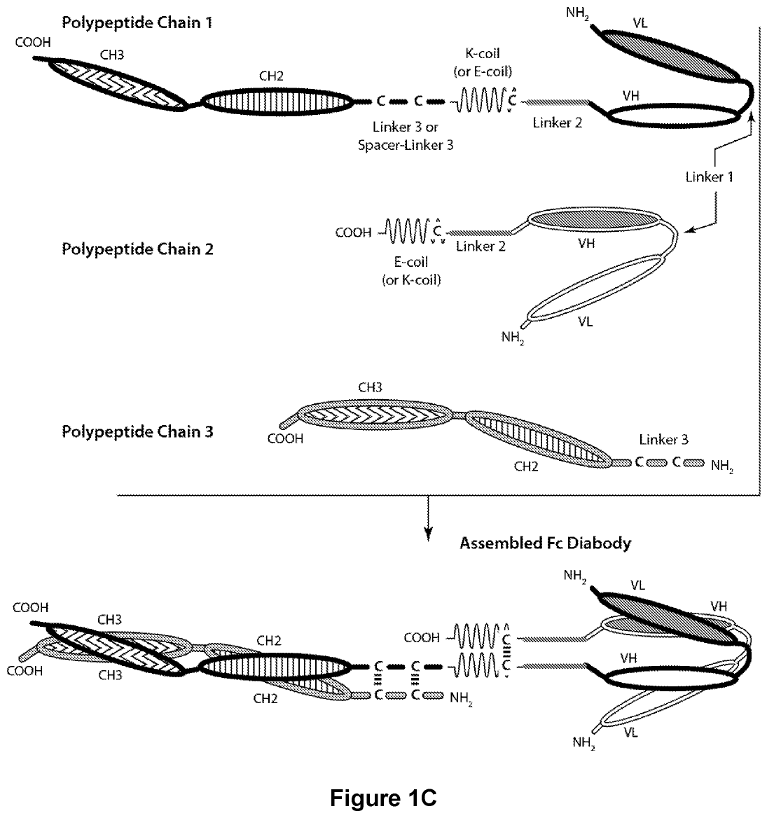

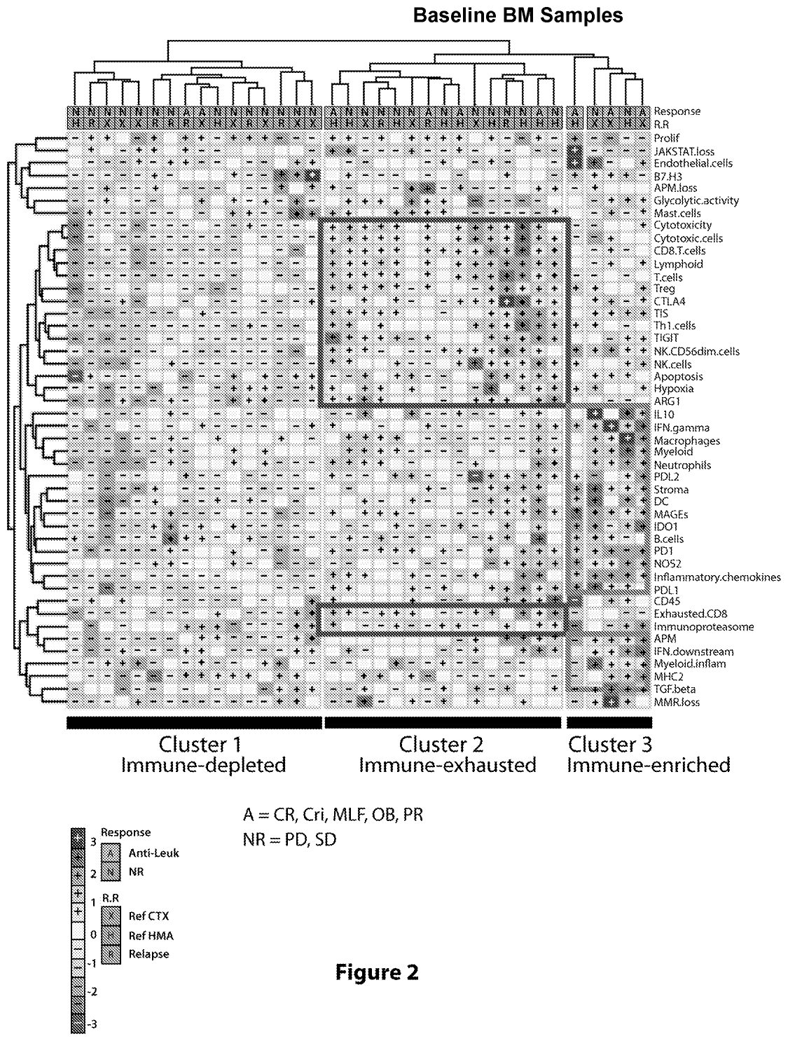

Within this group, the chemotherapy-refractory patients and the HMA-refractory patients further stratify into a first sub-population that exhibits gene signatures of an immune-exhausted tumor microenvironment (see, FIG. 2, boxed signatures indicated for Cluster 2) and a second sub-population that exhibits gene signatures of an immune-enriched tumor microenvironment including the Interferon Gamma (also referred to herein as “IFN gamma”) Signaling Signature (see, FIG. 2, boxed signatures indicated for Cluster 3). HMA-refractory patients display features of immune exhaustion and adaptive immune resistance, including an approximately 44% increase in TIGIT expression (5.55±0.34 vs 3.85±0.24, p=0.006), an approximately 48% increase in PD-L1 expression PD-L1 (3.55±0.18 vs 2.4±0.29, p=0.009) and an approximately 32% increase in Treg cell-specific expression (4.87±0.23 vs 3.69±0.19, p=0.0009) relative to chemotherapy-refractory patients. HMA-refractory patients also display a trend toward increasingly exhausted CD8 T cells (as measured by their expression of CD244, EOMES, LAG3 and PTGER4) compared to chemotherapy-refractory patients.

Focusing only on chemotherapy-refractory ((i.e., refractory to ≥2 induction attempts, first CR of <6 months) and relapsed AML patients (i.e., HMA-refractory patients were not included), further analysis of a broader set of genes (performed by aggregating the scores of three signature modules as described below) stratified relapsed and refractory AML patient BM samples at baseline into two immune subtypes, referred to herein as “immune-infiltrated” and “immune-depleted” subtypes.

### II. Exemplary CD123×CD3 Bispecific Binding Molecules

A. JNJ-63709178

JNJ-63709178 is a humanized IgG4 bispecific antibody with silenced Fc function. The antibody was produced using Genmab DuoBody® technology and is able to bind both CD123 on tumor cells and CD3 on T cells. JNJ-63709178 is able to recruit T cells to CD123-expressing tumor cells and induce the killing of these tumor cells in vitro (MOLM-13, OCI-AMLS and KG-1; EC50=0.51-0.91 nM). JNJ-63709178 is disclosed in WO 2016/036937, Gaudet, F. et al. (2016) “Development of a CD123×CD3 Bispecific Antibody (JNJ-63709178) for the Treatment of Acute Myeloid Leukemia (AML),” Blood 128:2824; and Forslund, A. et al. (2016) “Ex Vivo Activity Profile of the CD123×CD3 Duobody® Antibody JNI-63709178 Against Primary Acute Myeloid Leukemia Bone Marrow Samples,” Blood 128:2875, which documents are herein incorporated by reference). The amino acid sequences of the heavy and light chains of JNJ-63709178 and/or related antibodies: 13RB179, 13RB180, 13RB181, 13RB182, 13RB183, 13RB186, 13RB187, 13RB188, 13RB189, CD3B19, 7959, 3978, 7955, 9958, 8747, 8876, 4435 and 5466 are disclosed in WO 2016/036937.

B. XmAb14045

XmAb14045 (also known as vibecotamab) is a tumor-targeted antibody that contains both a CD123 binding domain and a cytotoxic T-cell binding domain (CD3). An XmAb Bispecific Fc domain serves as the scaffold for these two antigen binding domains and confers long circulating half-life, stability and ease of manufacture on XmAb14045. Engagement of CD3 by XmAb14045 activates T cells for highly potent and targeted killing of CD123-expressing tumor cells (US Patent Publication 2017/0349660; Chu, S. Y. et al. (2014) “Immunotherapy with Long-Lived Anti-CD123×CD3 Bispecific Antibodies Stimulates Potent T Cell-Mediated Killing of Human AML Cell Lines and of CD123+ Cells in Monkeys: A Potential Therapy for Acute Myelogenous Leukemia,” Blood 124(21):2316, which documents are herein incorporated by reference). The amino acid sequences of the heavy and light chains of XmAb14045 and similar CD123×CD3 bispecific binding molecules are disclosed in US Patent Publication 2017/0349660 and in WHO Drug Information, Proposed INN: List 120, 2018, 32(4):658-660.

C. APVO436

APVO436 is an ADAPTIR™ CD123×CD3 bispecific binding molecule that possesses an anti-CD123 scFv portion and an anti-CD3 scFv portion. Each of the scFv portions are bound to an Fc Domain that has been modified to abolish ADCC/CDC effector function. APVO436 is disclosed to bind human CD123 and CD3-expressing cells with EC₅₀ values in the low nM range and to demonstrate potent target-specific activity against CD123-expressing tumor cell lines at low effector to target ratios. APVO436 is disclosed to be capable of potently inducing endogenous T-cell activation and proliferation accompanied by depletion of CD123 expressing cells in experiments with primary AML subject samples and normal donor samples. APVO436 (see, Comeau, M. R. et al. (2018) “APVO436, a Bispecific anti-CD123×anti-CD3 ADAPTIR™ Molecule for Redirected T-cell Cytotoxicity, Induces Potent T-cell Activation, Proliferation and Cytotoxicity with Limited Cytokine Release,” AACR Annual Meeting April 2018, Abstract 1786; Godwin, C. D. et al. (2017) “Bispecific Anti-CD123×Anti-CD3 ADAPTIR™ Molecules APVO436 and APVO437 Have Broad Activity Against Primary Human AML Cells In Vitro,” American Society of Hematology Annual Meeting, December 2017, Blood 130:2639; Comeau, M. R. et al. (2017) “Bispecific anti-CD123×anti-CD3 ADAPTIR™ Molecules for Redirected T-cell Cytotoxicity in Hematological Malignancies,” AACR Annual Meeting April 2017, Abstract 597). The amino acid sequences of the heavy and light chains of APVO436 CD123×CD3 bispecific binding molecules are disclosed in WO 2018/057802A1.

D. DART-A

DART-A (also known as flotetuzumab, CAS number: 1664355-28-5) is the preferred CD123×CD3 bispecific binding molecule of the present invention. DART-A is a sequence-optimized bispecific diabody capable of simultaneously and specifically binding to an epitope of CD123 and to an epitope of CD3 (a “CD123×CD3” bispecific diabody) (US Patent Publn. No. US 2016-0200827, in PCT Publn. WO 2015/026892, in Al-Hussaini, M. et al. (2016) “Targeting CD123 In Acute Myeloid Leukemia Using A T-Cell-Directed Dual-Affinity Retargeting Platform,” Blood 127:122-131, in Vey, N. et al. (2017) “A Phase 1, First-in-Human Study of MGD006/S80880 (CD123×CD3) in AML/MDS,” 2017 ASCO Annual Meeting, Jun. 2-6, 2017, Chicago, Ill.: Abstract TPS7070, each of which documents is herein incorporated by reference in its entirety). DART-A was found to exhibit enhanced functional activity relative to other non-sequence-optimized CD123×CD3 bispecific diabodies of similar composition, and is thus termed a “sequence-optimized” CD123×CD3 bispecific diabody. PCT Application PCT/US2017/050471 describes preferred dosing regimens for administering DART-A to patients, and is herein incorporated by reference in its entirety.

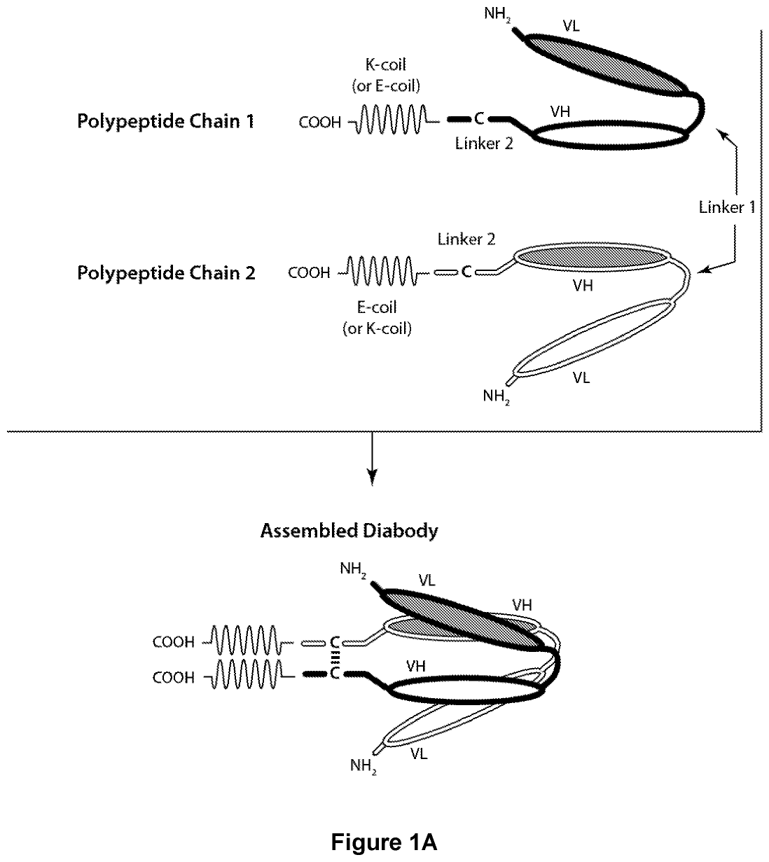

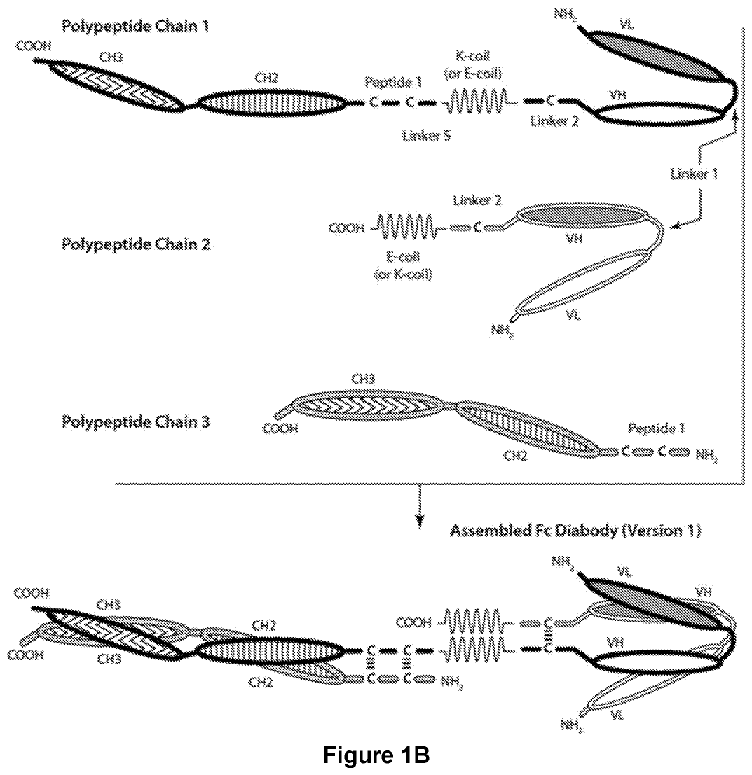

DART-A comprises a first polypeptide chain and a second polypeptide chain (FIG. 1). The first polypeptide chain of the bispecific diabody will comprise, in the N-terminal to C-terminal direction, an N-terminus, a Light Chain Variable Domain (VL Domain) of a monoclonal antibody capable of binding to CD3 (VL_{CD3}), an intervening linker peptide (Linker 1), a Heavy Chain Variable Domain (VH Domain) of a monoclonal antibody capable of binding to CD123 (VH_{CD123}), and a C-terminus.

A preferred sequence for such a VL_{CD3} Domain is SEQ ID NO:1:

QAVVTQEPSL TVSPGGTVTL TCRSSTGAVT
TSNYANWVQQ KPGQAPRGLI GGTNKRAPWT
PARFSGSLLG GKAALTITGA QAEDEADYYC
ALWYSNLWVF GGGTKLTVLG

The Antigen Binding Domain of VL_{CD3} comprises:

CDR_{L}1 (SEQ ID NO: 2):
RSSTGAVTTSNYAN
CDR_{L}2 (SEQ ID NO: 3):
GTNKRAP
CDR_{L}3 (SEQ ID NO: 4):
ALWYSNLWV

A preferred sequence for such Linker 1 is SEQ ID NO:5: GGGSGGGG. A preferred sequence for such a VH_{CD123} Domain is SEQ ID NO:6:

EVQLVQSGAE LKKPGASVKV SCKASGYTFT
DYYMKWVRQA PGQGLEWIGD IIPSNGATFY
NQKFKGRVTI TVDKSTSTAY MELSSLRSED
TAVYYCARSH LLRASWFAYW GQGTLVTVSS

The Antigen Binding Domain of VH_{CD123} comprises:

CDR_{H}1 (SEQ ID NO: 7):
DYYMK
CDR_{H}2 (SEQ ID NO: 8):
DIIPSNGATFYNQKFKG
CDR_{H}3 (SEQ ID NO: 9):
SHLLRASWFAY

The second polypeptide chain will comprise, in the N-terminal to C-terminal direction, an N-terminus, a VL domain of a monoclonal antibody capable of binding to CD123 (VL_{CD123}), an intervening linker peptide (e.g., Linker 1), a VH domain of a monoclonal antibody capable of binding to CD3 (VH_{CD3}), and a C-terminus. A preferred sequence for such a VL_{CD123} Domain is SEQ ID NO:10:

DFVMTQSPDS LAVSLGERVT MSCKSSQSLL
NSGNQKNYLT WYQQKPGQPP KLLIYWASTR
ESGVPDRFSG SGSGTDFTLT ISSLQAEDVA
VYYCQNDYSY PYTFGQGTKL EIK

The Antigen Binding Domain of VL_{CD123} comprises:

CDR_{L}1 (SEQ ID NO: 11):
KSSQSLLNSGNQKNYLT
CDR_{L}2 (SEQ ID NO: 12):
WASTRES
CDR_{L}3 (SEQ ID NO: 13):
QNDYSYPYT

A preferred sequence for such a VH_{CD3} Domain is SEQ ID NO:14:

EVQLVESGGG LVQPGGSLRL SCAASGFTFS
TYAMNWVRQA PGKGLEWVGR IRSKYNNYAT
YYADSVKDRF TISRDDSKNS LYLQMNSLKT
EDTAVYYCVR HGNFGNSYVS WFAYWGQGTL
VTVSS

The Antigen Binding Domain of VH_{CD3} comprises:

CDR_{H}1 (SEQ ID NO: 15):
TYAMN
CDR_{H}2 (SEQ ID NO: 16):
RIRSKYNNYATYYADSVKD
CDR_{H}3 (SEQ ID NO: 17):
HGNFGNSYVSWFAY

The sequence-optimized CD123×CD3 bispecific diabodies of the present invention are engineered so that such first and second polypeptides covalently bond to one another via cysteine residues along their length. Such cysteine residues may be introduced into the intervening linker (e.g., Linker 1) that separates the VL and VH domains of the polypeptides. Alternatively, and more preferably, a second peptide (Linker 2) is introduced into each polypeptide chain, for example, at a position N-terminal to the VL domain or C-terminal to the VH domain of such polypeptide chain. A preferred sequence for such Linker 2 is SEQ ID NO:18: GGCGGG.

The formation of heterodimers can be driven by further engineering such polypeptide chains to contain polypeptide coils of opposing charge. Thus, in a preferred embodiment, one of the polypeptide chains will be engineered to contain an “E-coil” domain (SEQ ID NO:19: EVAALEKEVAALEKEVAALEKEVAALEK) whose residues will form a negative charge at pH 7, while the other of the two polypeptide chains will be engineered to contain an “K-coil” domain (SEQ ID NO:20: KVAALKEKVAALKEKVAALKEKVAALKE) whose residues will form a positive charge at pH 7. The presence of such charged domains promotes association between the first and second polypeptides, and thus fosters heterodimerization.

It is immaterial which coil is provided to the first or second polypeptide chains. However, a preferred sequence-optimized CD123×CD3 bispecific diabody of the present invention (“DART-A”) has a first polypeptide chain having the sequence (SEQ ID NO:21):

QAVVTQEPSL TVSPGGTVTL TCRSSTGAVT
TSNYANWVQQ KPGQAPRGLI GGTNKRAPWT
PARFSGSLLG GKAALTITGA QAEDEADYYC
ALWYSNLWVF GGGTKLTVLG GGGSGGGGEV
QLVQSGAELK KPGASVKVSC KASGYTFTDY
YMKWVRQAPG QGLEWIGDII PSNGATFYNQ
KFKGRVTITV DKSTSTAYME LSSLRSEDTA
VYYCARSHLL RASWFAYWGQ GTLVTVSSGG
CGGGEVAALE KEVAALEKEV AALEKEVAAL
EK

DART-A Chain 1 is composed of: SEQ ID NO:1-SEQ ID NO:5-SEQ ID NO:6-SEQ ID NO:18-SEQ ID NO:19. A polynucleotide that encodes the first polypeptide chain of DART-A is SEQ ID NO:22:

caggctgtgg tgactcagga gccttcactg
accgtgtccc caggcggaac tgtgaccctg
acatgcagat ccagcacagg cgcagtgacc
acatctaact acgccaattg ggtgcagcag
aagccaggac aggcaccaag gggcctgatc
gggggtacaa acaaaagggc tccctggacc
cctgcacggt tttctggaag tctgctgggc
ggaaaggccg ctctgactat taccggggca
caggccgagg acgaagccga ttactattgt
gctctgtggt atagcaatct gtgggtgttc
gggggtggca caaaactgac tgtgctggga
gggggtggat ccggcggcgg aggcgaggtg
cagctggtgc agtccggggc tgagctgaag
aaacccggag cttccgtgaa ggtgtcttgc
aaagccagtg gctacacctt cacagactac
tatatgaagt gggtcaggca ggctccagga
cagggactgg aatggatcgg cgatatcatt
ccttccaacg gggccacttt ctacaatcag
aagtttaaag gcagggtgac tattaccgtg
gacaaatcaa caagcactgc ttatatggag
ctgagctccc tgcgctctga agatacagcc
gtgtactatt gtgctcggtc acacctgctg
agagccagct ggtttgctta ttggggacag
ggcaccctgg tgacagtgtc ttccggagga
tgtggcggtg gagaagtggc cgcactggag
aaagaggttg ctgctttgga gaaggaggtc
gctgcacttg aaaaggaggt cgcagccctg
gagaaa

The second polypeptide chain of DART-A has the sequence (SEQ ID NO:23):

DFVMTQSPDS LAVSLGERVT MSCKSSQSLL
NSGNQKNYLT WYQQKPGQPP KLLIYWASTR
ESGVPDRFSG SGSGTDFTLT ISSLQAEDVA
VYYCQNDYSY PYTFGQGTKL EIKGGGSGGG
GEVQLVESGG GLVQPGGSLR LSCAASGFTF
STYAMNWVRQ APGKGLEWVG RIRSKYNNYA
TYYADSVKDR FTISRDDSKN SLYLQMNSLK
TEDTAVYYCV RHGNFGNSYV SWFAYWGQGT
LVTVSSGGCG GGKVAALKEK VAALKEKVAA
LKEKVAALKE

DART-A Chain 2 is composed of: SEQ ID NO:10-SEQ ID NO:5-SEQ ID NO:14-SEQ ID NO:18-SEQ ID NO:20. A polynucleotide that encodes the second polypeptide chain of DART-A is SEQ ID NO:24:

gacttcgtga tgacacagtc tcctgatagt
ctggccgtga gtctggggga gcgggtgact
atgtcttgca agagctccca gtcactgctg
aacagcggaa atcagaaaaa ctatctgacc
tggtaccagc agaagccagg ccagccccct
aaactgctga tctattgggc ttccaccagg
gaatctggcg tgcccgacag attcagcggc
agcggcagcg gcacagattt taccctgaca
atttctagtc tgcaggccga ggacgtggct
gtgtactatt gtcagaatga ttacagctat
ccctacactt tcggccaggg gaccaagctg
gaaattaaag gaggcggatc cggcggcgga
ggcgaggtgc agctggtgga gtctggggga
ggcttggtcc agcctggagg gtccctgaga
ctctcctgtg cagcctctgg attcaccttc
agcacatacg ctatgaattg ggtccgccag
gctccaggga aggggctgga gtgggttgga
aggatcaggt ccaagtacaa caattatgca
acctactatg ccgactctgt gaaggataga
ttcaccatct caagagatga ttcaaagaac
tcactgtatc tgcaaatgaa cagcctgaaa
accgaggaca cggccgtgta ttactgtgtg
agacacggta acttcggcaa ttcttacgtg
tcttggtttg cttattgggg acaggggaca
ctggtgactg tgtcttccgg aggatgtggc
ggtggaaaag tggccgcact gaaggagaaa
gttgctgctt tgaaagagaa ggtcgccgca
cttaaggaaa aggtcgcagc cctgaaagag

DART-A has the ability to simultaneously bind CD123 and CD3 as arrayed by human and cynomolgus monkey cells. Provision of DART-A was found to cause T cell activation, to mediate blast reduction, to drive T cell expansion, to induce T cell activation and to cause the redirected killing of target cancer cells (Table 4).

TABLE 4
Equilibrium Dissociation Constants (K_{D}) for the Binding
of DART-A to Human and Cynomolgus Monkey CD3 and CD123

| | kₐ (±SD) | k_{d} (±SD) | K_{D} (±SD) |
|---|---|---|---|
| Antigens | (M⁻¹s⁻¹) | (s⁻¹) | (nM) |
| Human CDε/δ | 5.7 (±0.6) × 10⁵ | 5.0 (±0.9) × 10⁻³ | 9.0 ± 2.3 |
| Cynomolgus | 5.5 (±0.5) × 10⁵ | 5.0 (±0.9) × 10⁻³ | 9.2 ± 2.3 |
| CD3ε/δ | | | |
| Human CD123-His | 1.6 (±0.4) × 10⁶ | 1.9 (±0.4) × 10⁻⁴ | 0.13 ± 0.01 |
| Cynomolgus | 1.5 (±0.3) × 10⁶ | 4.0 (±0.7) × 10⁻⁴ | 0.27 ± 0.02 |
| CD123-His | | | |

More particularly, DART-A exhibits a potent redirected killing ability with concentrations required to achieve 50% of maximal activity (EC50s) in sub-ng/mL range, regardless of CD3 epitope binding specificity in target cell lines with high CD123 expression (Kasumi-3 (EC₅₀=0.01 ng/mL)) medium CD123-expression (Molm13 (EC₅₀=0.18 ng/mL) and THP-1 (EC₅₀=0.24 ng/mL)) and medium low or low CD123 expression (TF-1 (EC₅₀=0.46 ng/mL) and RS4-11 (EC₅₀=0.5 ng/mL)). Similarly, DART-A-redirected killing was also observed with multiple target cell lines with T cells from different donors and no redirected killing activity was observed in cell lines that do not express CD123. Results are summarized in Table 5.

TABLE 5

| | CD123 Surface | EC₅₀ of Sequence- | |
|---|---|---|---|
| Target | Expression | Optimized CD 123 × CD3 | |
| Cell | (Antibody | Bispecific Diabodies (ng/mL) | Max % |
| Line | Binding Sites) | E:T = 10:1 | Killing |
| Kasumi-3 | 118620 | 0.01 | 94 |
| Molm13 | 27311 | 0.18 | 43 |
| THP-1 | 58316 | 0.24 | 40 |
| TF-1 | 14163 | 0.46 | 46 |
| RS4-11 | 957 | 0.5 | 60 |
| A498 | Negative | No activity | No activity |
| HT29 | Negative | No activity | No activity |

Additionally, when human T cells and tumor cells (Molm13 or RS4-11) were combined and injected subcutaneously into NOD/SCID gamma (NSG) knockout mice, the MOLM13 tumors was significantly inhibited at the 0.16, 0.5, 0.2, 0.1, 0.02, and 0.004 mg/kg dose levels. A dose of 0.004 mg/kg and higher was active in the MOLM13 model. The lower DART-A doses associated with the inhibition of tumor growth in the MOLM13 model compared with the RS4-11 model are consistent with the in vitro data demonstrating that MOLM13 cells have a higher level of CD123 expression than RS4-11 cells, which correlated with increased sensitivity to DART-A-mediated cytotoxicity in vitro in MOLM13 cells.

DART-A is active against primary AML specimens (bone marrow mononucleocytes (BMNC) and peripheral blood mononucleocytes (PBMC)) from AML patients. Incubation of primary AML bone marrow samples with DART-A resulted in depletion of the leukemic cell population over time, accompanied by a concomitant expansion of the residual T cells (both CD4 and CD8) and the induction of T cell activation markers (CD25 and Ki-67). Upregulation of granzyme B and perform levels in both CD8 and CD4 T cells was observed. Incubation of primary AML bone marrow samples with DART-A resulted in depletion of the leukemic cell population over time compared to untreated control or Control DART. When the T cells were counted (CD8 and CD4 staining) and activation (CD25 staining) were assayed, the T cells expanded and were activated in the DART-A sample compared to untreated or Control DART samples. DART-A was also found to be capable of mediating the depletion of pDCs cells in both human and cynomolgus monkey PBMCs, with cynomolgus monkey pDCs being depleted as early as 4 days post infusion with as little as 10 ng/kg DART-A. No elevation in the levels of cytokines interferon gamma, TNF alpha, IL6, IL5, IL4 and IL2 were observed in DART-A-treated animals. These data indicate that DART-A-mediated target cell killing was mediated through a granzyme B and perform pathway.

No activity was observed against CD123-negative targets (U937 cells) or with Control DART, indicating that the observed T cell activation was strictly dependent upon target cell engagement and that monovalent engagement of CD3 by DART-A was insufficient to trigger T cell activation.

In sum, DART-A is an antibody-based molecule engaging the CD3ε subunit of the TCR to redirect T lymphocytes against cells expressing CD123, an antigen up-regulated in several hematologic malignancies. DART-A binds to both human and cynomolgus monkey's antigens with similar affinities and redirects T cells from both species to kill CD123+ cells. Monkeys infused 4 or 7 days a week with weekly escalating doses of DART-A showed depletion of circulating CD123+ cells 72 h after treatment initiation that persisted throughout the 4 weeks of treatment, irrespective of dosing schedules. A decrease in circulating T cells also occurred, but recovered to baseline before the subsequent infusion in monkeys on the 4-day dose schedule, consistent with DART-A-mediated mobilization. DART-A administration increased circulating PD1+, but not TIM-3+, T cells; furthermore, ex vivo analysis of T cells from treated monkeys exhibited unaltered redirected target cell lysis, indicating no exhaustion. Toxicity was limited to a minimal transient release of cytokines following the DART-A first infusion, but not after subsequent administrations even when the dose was escalated, and a minimal reversible decrease in red cell mass with concomitant reduction in CD123+ bone marrow progenitors.

E. Additional Bispecific Diabody Molecules

An alternative version of DART-A comprising an Fc Region and having the general structure shown in FIG. 1B is described in US 2016-0200827. Preferred polypeptides that contains the CH2 and CH3 Domains of an Fc Domain have the sequence (SEQ ID NO:25) (“Knob-Bearing” Fc Domain):

APEAAGGPSV FLFPPKPKDT LMISRTPEVT
CVVVDVSHED PEVKFNWYVD GVEVHNAKTK
PREEQYNSTY RVVSVLTVLH QDWLNGKEYK
CKVSNKALPA PIEKTISKAK GQPREPQVYT
LPPSREEMTK NQVSLWCLVK GFYPSDIAVE
WESNGQPENN YKTTPPVLDS DGSFFLYSKL
TVDKSRWQQG NVFSCSVMHE ALHNHYTQKS
LSLSPGX
wherein X is K or is absent

and the sequence (SEQ ID NO:26) (“Hole-Bearing” Fc Domain):

APEAAGGPSV FLFPPKPKDT LMISRTPEVT
CVVVDVSHED PEVKFNWYVD GVEVHNAKTK
PREEQYNSTY RVVSVLTVLH QDWLNGKEYK
CKVSNKALPA PIEKTISKAK GQPREPQVYT
LPPSREEMTK NQVSLSCAVK GFYPSDIAVE
WESNGQPENN YKTTPPVLDS DGSFFLVSKL
TVDKSRWQQG NVFSCSVMHE ALHNRYTQKS
LSLSPGX
wherein X is K or is absent

The first polypeptide of an exemplary DART-A w/Fc construct comprises, in the N-terminal to C-terminal direction, an N-terminus, a VL domain of a monoclonal antibody capable of binding to CD123 (VL_{CD123}), an intervening linker peptide (Linker 1), a VH domain of a monoclonal antibody capable of binding to CD3 (VH_{CD3}), a Linker 2, an E-coil Domain, a Linker 5, Peptide 1, a polypeptide that contains the CH2 and CH3 Domains of an Fc Domain and a C-terminus. A preferred Linker 5 has the sequence: GGG. A preferred Peptide 1 has the sequence: DKTHTCPPCP (SEQ ID NO:29). Thus, the first polypeptide of such a DART-A w/Fc version 1 construct is composed of: SEQ ID NO:10-SEQ ID NO:5-SEQ ID NO:14-SEQ ID NO:18-SEQ ID NO:19-GGG-SEQ ID NO:29-SEQ ID NO:25 (wherein X is K).

A preferred sequence of the first polypeptide of such a DART-A w/Fc version 1 construct has the sequence (SEQ ID NO:27):

DFVMTQSPDS LAVSLGERVT MSCKSSQSLL
NSGNQKNYLT WYQQKPGQPP KLLIYWASTR
ESGVPDRFSG SGSGTDFTLT ISSLQAEDVA
VYYCQNDYSY PYTFGQGTKL EIKGGGSGGG
GEVQLVESGG GLVQPGGSLR LSCAASGFTF
STYAMNWVRQ APGKGLEWVG RIRSKYNNYA
TYYADSVKDR FTISRDDSKN SLYLQMNSLK
TEDTAVYYCV RHGNFGNSYV SWFAYWGQGT
LVTVSSGGCG GGEVAALEKE VAALEKEVAA
LEKEVAALEK GGGDKTHTCP PCPAPEAAGG
PSVFLFPPKP KDTLMISRTP EVTCVVVDVS
HEDPEVKFNW YVDGVEVHNA KTKPREEQYN
STYRVVSVLT VLHQDWLNGK EYKCKVSNKA
LPAPIEKTIS KAKGQPREPQ VYTLPPSREE
MTKNQVSLWC LVKGFYPSDI AVEWESNGQP
ENNYKTTPPV LDSDGSFFLY SKLTVDKSRW
QQGNVFSCSV MHEALHNHYT QKSLSLSPGK

The second chain of such a DART-A w/Fc version 1 construct will comprise, in the N-terminal to C-terminal direction, an N-terminus, a VL domain of a monoclonal antibody capable of binding to CD3 (VL_{CD3}), an intervening linker peptide (Linker 1), a VH domain of a monoclonal antibody capable of binding to CD123 (VH_{CD123}), a Linker 2, a K-coil Domain, and a C-terminus. Thus, the second polypeptide of such a DART-A w/Fc version 1 construct is composed of: SEQ ID NO:1-SEQ ID NO:5-SEQ ID NO:6-SEQ ID NO:18-SEQ ID NO:20. Such a polypeptide has the sequence (SEQ ID NO:28):

QAVVTQEPSL TVSPGGTVTL TCRSSTGAVT
TSNYANWVQQ KPGQAPRGLI GGTNKRAPWT
PARFSGSLLG GKAALTITGA QAEDEADYYC
ALWYSNLWVF GGGTKLTVLG GGGSGGGGEV
QLVQSGAELK KPGASVKVSC KASGYTFTDY
YMKWVRQAPG QGLEWIGDII PSNGATFYNQ
KFKGRVTITV DKSTSTAYME LSSLRSEDTA
VYYCARSHLL RASWFAYWGQ GTLVTVSSGG
CGGGKVAALK EKVAALKEKV AALKEKVAAL
KE

The third polypeptide chain of such a DART-A w/Fc version 1 will comprise the CH2 and CH3 Domains of an IgG Fc Domain. A preferred polypeptide that is composed of Peptide 1 (DKTHTCPPCP; SEQ ID NO:29) and the CH2 and CH3 Domains of an Fc Domain (SEQ ID NO:26, wherein X is K) and has the sequence of SEQ ID NO:30:

DKTHTCPPCP APEAAGGPSV FLFPPKPKDT
LMISRTPEVT CVVVDVSHED PEVKFNWYVD
GVEVHNAKTK PREEQYNSTY RVVSVLTVLH
QDWLNGKEYK CKVSNKALPA PIEKTISKAK
GQPREPQVYT LPPSREEMTK NQVSLSCAVK
GFYPSDIAVE WESNGQPENN YKTTPPVLDS
DGSFFLVSKL TVDKSRWQQG NVFSCSVMHE
ALHNRYTQKS LSLSPGK

Additional CD123×CD3 bispecific diabodies comprising alternative optimized anti-CD3 binding domains are provided in U.S. Application Nos.: 62/631,043 (filed on Feb. 15, 2018); and 62/738,632 (filed on Sep. 28, 2018) (all of which are incorporated herein).

### III. Pharmaceutical Formulations

The compositions of the invention include bulk drug compositions useful in the manufacture of pharmaceutical compositions (e.g., impure or non-sterile compositions) and pharmaceutical compositions (i.e., compositions that are suitable for administration to a subject or patient) which can be used in the preparation of unit dosage forms. Such compositions comprise a prophylactically or therapeutically effective amount of a CD123×CD3 bispecific binding molecule and a pharmaceutically acceptable carrier.

Preferred pharmaceutical formulations comprise a CD123×CD3 bispecific binding molecule and an aqueous stabilizer and, optionally, a pharmaceutically acceptable carrier.

As used herein, the term “pharmaceutically acceptable carrier” is intended to refer to a diluent, adjuvant (e.g., Freund's adjuvant (complete and incomplete)), excipient, or vehicle that is approved by a regulatory agency or listed in the U.S. Pharmacopeia or in another generally recognized pharmacopeia as being suitable for delivery into animals, and more particularly, humans. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like.

Generally, the ingredients of compositions of the invention are supplied either separately or mixed together in unit dosage form, for example, as a liquid formulation, as a dry lyophilized powder or water free concentrate in a hermetically sealed container such as a vial, an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The invention also provides a pharmaceutical pack or kit comprising one or more containers containing a CD123×CD3 bispecific binding molecule alone or with a stabilizer and/or a pharmaceutically acceptable carrier. Additionally, one or more other prophylactic or therapeutic agents useful for the treatment of a disease can also be included in the pharmaceutical pack or kit. The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

### IV. Kits

The present invention provides kits that comprise a CD123×CD3 bispecific binding molecule, instructional material (for example, relating to storage, dosage, indications, side effects, counter-indications, etc.), and optionally a stabilizer and/or carrier that can be used in the above methods. In such kits, the CD123×CD3 bispecific binding molecule is preferably packaged in a hermetically sealed container such as an ampoule, a vial, a sachette, etc. that preferably indicates the quantity of the molecule contained therein. The container may be formed of any pharmaceutically acceptable material, such as glass, resin, plastic, etc. The CD123×CD3 bispecific binding molecule of such kit is preferably supplied as a liquid solution, a dry sterilized lyophilized powder or a water-free concentrate in a hermetically sealed container that can be reconstituted, e.g., with water or saline to the appropriate concentration for administration to a subject. Such liquid or lyophilized material should be stored at between 2 and 8° C. in its original container and the material should be administered within 12 hours, preferably within 6 hours, within 5 hours, within 3 hours, or within 1 hour after being reconstituted. The kit can further comprise one or more other prophylactic and/or therapeutic agents useful for the treatment of cancer, in one or more containers; and/or the kit can further comprise one or more cytotoxic antibodies that bind one or more cancer antigens associated with cancer. In certain embodiments, the other prophylactic or therapeutic agent is a chemotherapeutic. In other embodiments, the prophylactic or therapeutic agent is a biological or hormonal therapeutic. The kit can further comprise instructions for use, or other printed information.

Additionally, one or more other prophylactic or therapeutic agents useful for the treatment of a disease can also be included in the pharmaceutical pack or kit. The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

### V. Methods of Administration

The CD123×CD3 bispecific binding molecule pharmaceutical formulations of the present invention may be provided for the treatment, prophylaxis, and amelioration of one or more symptoms associated with a disease, disorder or infection by administering to a subject an effective amount of a molecule of the invention, or a pharmaceutical composition comprising a fusion protein or a conjugated molecule of the invention. In a preferred aspect, such compositions are substantially purified (i.e., substantially free from substances that limit its effect or produce undesired side effects). In a specific embodiment, the subject is an animal, preferably a mammal such as non-primate (e.g., bovine, equine, feline, canine, rodent, etc.) or a primate (e.g., monkey such as, a cynomolgus monkey, human, etc.). In a preferred embodiment, the subject or patient is a human.

Methods of administering a CD123×CD3 bispecific binding molecule pharmaceutical formulation of the invention include, but are not limited to, parenteral administration (e.g., intradermal, intramuscular, intraperitoneal, intravenous and subcutaneous). In a specific embodiment, the CD123×CD3 bispecific binding molecules are administered intravenously. The compositions may be administered by any convenient route, for example, by infusion, and may be administered together with other biologically active agents.

Administration by infusion is preferably accomplished using an infusion pump. “Infusion pumps” are medical device that deliver fluids into a patient's body in a controlled manner, especially at a defined rate and for a prolonged period of time. Infusion pumps may be powered mechanically, but are more preferably electrically powered. Some infusion pumps are “stationary” infusion pumps, and are designed to be used at a patient's bedside. Others, called “ambulatory” infusion pumps, are designed to be portable or wearable. A “syringe” pump is an infusion pump in which the fluid to be delivered is held in the reservoir of a chamber (e.g., a syringe), and a moveable piston is used to control the chamber's volume and thus the delivery of the fluid. In an “elastomeric” infusion pump, fluid is held in a stretchable balloon reservoir, and pressure from the elastic walls of the balloon drives fluid delivery. In a “peristaltic” infusion pump, a set of rollers pinches down on a length of flexible tubing, pushing fluid forward. In a “multi-channel” infusion pump, fluids can be delivered from multiple reservoirs at multiple rates. A “smart pump” is an infusion pump that is equipped a computer-controlled fluid delivery system so as to be capable of alerting in response to a risk of an adverse drug interaction, or when the pump's parameters have been set beyond specified limits. Examples of infusion pumps are well-known, and are provided in, for example, [Anonymous] 2002 “General-Purpose Infusion Pumps,” Health Devices 31(10):353-387; and in U.S. Pat. Nos. 10,029,051, 10,029,047, 10,029,045, 10,022,495, 10,022,494, 10,016,559, 10,006,454, 10,004,846, 9,993,600, 9,981,082, 9,974,901, 9,968,729, 9,931,463, 9,927,943, etc.

It is preferred that the CD123×CD3 bispecific binding molecule pharmaceutical formulations of the invention be administered by infusion facilitated by one or more ambulatory pumps, so that the patient will be ambulatory during the therapeutic regimen. It is preferred that the CD123×CD3 bispecific binding molecule pharmaceutical formulations of the invention be administered by continuous infusion. In a preferred embodiment, a 7-day continuous infusion regimen comprises a treatment dosage of about 30 ng/kg patient weight/day for 3 days followed by a treatment dosage of about 100 ng/kg/day for 4 days (for example, a treatment dosage of 30 ng/kg patient weight/day for 3 days followed by a treatment dosage of 100 ng/kg/day for 4 days; etc.). In particularly preferred embodiments, such 7-day continuous infusion regiment is followed by a 21-day continuous infusion regiment in which a treatment dosage of 500 ng/kg/day is administered during days 1˜4 of each week of such 21-day regiment and during days 5-7 of each week no treatment dosage is administered. Alternatively, such 7-day continuous infusion regiment is followed by a 21-day continuous infusion regiment in which a treatment dosage of 500 ng/kg/day is administered every day for 21 days.

In any of the above-described courses of treatment, the proportion of CD8+ T-lymphocytes in the tumor microenvironment may additionally be monitored. Such monitoring may occur prior to the administration of the CD123×CD3 bispecific binding molecule, during the course of CD123×CD3 binding molecule therapy, and/or after the conclusion of a cycle of CD123×CD3 binding molecule therapy.

### VI. Uses of the Compositions of the Invention

The CD123×CD3 bispecific binding molecules of the invention may be used to treat any disease or condition associated with or characterized by the expression of CD123. In particular, the CD123×CD3 bispecific binding molecules of the invention may be used to treat hematologic malignancies. The CD123×CD3 bispecific binding molecules of the invention are particularly suitable for use in the treatment of hematologic malignancies, including chemo-refractory hematologic malignancies. As used herein, a chemo-refractory hematologic malignancy is a hematologic malignancy that is refractory to two or more induction attempts, a first CR of less than 6 months, or a failure after two or more cycles of treatment with a hypomethylating agent).

Thus, without limitation, such molecules may be employed in the diagnosis or treatment of acute myeloid leukemia (AML) (including primary chemo-refractory AML), chronic myelogenous leukemia (CML), including blastic crisis of CIVIL and Abelson oncogene-associated with CIVIL (Bcr-ABL translocation), myelodysplastic syndrome (MDS), acute B lymphoblastic leukemia (B-ALL), acute T lymphoblastic leukemia (T-ALL), chronic lymphocytic leukemia (CLL), including Richter's syndrome or Richter's transformation of call, hairy cell leukemia (HCL), blastic plasmacytoid dendritic cell neoplasm (BPDCN), non-Hodgkin's lymphoma (NHL), including mantle cell lymphoma (MCL) and small lymphocytic lymphoma (SLL), Hodgkin's lymphoma, systemic mastocytosis, and Burkitt's lymphoma. The CD123×CD3 bispecific binding molecules of the invention may additionally be used in the manufacture of medicaments for the treatment of the above-described conditions.

The CD123×CD3 bispecific binding molecules of the invention are particularly suitable for use in the treatment of acute myeloid leukemia (AML, including primary chemo-refractory acute myeloid leukemia), hematologic myelodysplastic syndrome (MDS), blastic plasmacytoid dendritic cell neoplasm (BPDCN), non-Hodgkin's lymphoma (NHL), or acute T lymphoblastic leukemia (T-ALL).

### VII. Particular Embodiments of the Invention

Having now generally described the invention, the same will be more readily understood through reference to the following numbered Embodiments (“E1”-“E60”), which are provided by way of illustration only and are not intended to be limiting of the present invention, unless specified:
E1. A method of treating a chemo-refractory hematologic malignancy in a patient, wherein said method comprises administering to said patient a treatment dosage of a CD123×CD3 bispecific molecule, said dosage being effective to stimulate the killing of cells of said hematologic malignancy in said patient and thereby treat said malignancy.
E2. The method of E1, wherein said method additionally comprises evaluating the expression of one or more target and/or reference genes in a cellular sample from said patient, prior to and/or subsequent to said administration of said CD123×CD3 bispecific molecule.
E3. The method of E2, wherein said method comprises evaluating the expression of said one or more target and/or said one or more reference genes prior to said administration of said CD123×CD3 bispecific molecule.
E4. The method of E2, wherein said method comprises evaluating the expression of said one or more target and/or said one or more reference genes subsequent to said administration of said CD123×CD3 bispecific molecule.
E5. A method of determining whether a patient would be a suitable responder to the use of a CD123×CD3 bispecific molecule to treat a hematologic malignancy, wherein said method comprises:
   (a) evaluating the expression of one or more target genes in a cellular sample from said patient prior to the administration of said CD123×CD3 bispecific molecule, relative to the expression of one or more target and/or reference genes; and
   (b) identifying the patient as a suitable responder for treatment with a CD123×CD3 bispecific molecule if the expression of said one or more target genes is found to be increased relative to said expression of said one or more target and/or reference genes.
E6. The method of any one of E2-E6, wherein said method evaluates:
   (i) the expression of one or more target genes; and
   (ii) one or more reference genes whose expression is not characteristically associated with said hematologic malignancy.
E7. The method of any one of E2-E6, wherein said method comprises evaluating the expression of said one or more target genes relative to the baseline expression of said one or more reference genes of said patient.
E8. The method of any one of E2-E7, wherein said method comprises evaluating the expression of said one or more target genes of said patient relative to the expression of said one or more target genes of an individual who is suffering from said hematologic malignancy, or of a population of such individuals.
E9. The method of any one of E2-E7, wherein said method comprises evaluating the expression of said one or more target genes of said patient relative to the expression of said one or more target genes of an individual who did not successfully respond to the use of a CD123×CD3 bispecific molecule to treat said hematologic malignancy, or of a population of such individuals.
E10. The method of any one of E2-E7, wherein said method comprises evaluating the expression of said one or more target genes of said patient relative to the expression of said one or more target genes of an individual who successfully responded to the use of a CD123×CD3 bispecific molecule to treat said hematologic malignancy, or of a population of such individuals.
E11. The method of any one of E7-E10, wherein the relative expression level of said one or more target genes in said population is established by averaging the gene expression level in cellular samples obtained from said population of individuals.
E12. The method of any one of E2-E11, wherein said patient exhibits an expression level of at least one of said target genes:
   (a) that is greater than the first quartile of the expression levels of said target gene in a population of individuals who are suffering from said hematologic malignancy; or
   (b) that is greater than the first quartile of the expression levels of said target gene in a population of individuals who did not successfully respond to a treatment for said hematologic malignancy that used a CD123×CD3 bispecific molecule; or
   (c) that has a log₂-fold change of at least about 0.4 relative to the expression levels of said target gene in a population of individuals who did not successfully respond to a treatment for said hematologic malignancy that used a CD123×CD3 bispecific molecule; or
   (d) that is within at least the first quartile of the expression levels of said target gene in a population of individuals who successfully responded to a treatment for said hematologic malignancy that used a CD123×CD3 bispecific molecule.
E13. The method of any one of E2-E11, wherein said patient exhibits an expression level of at least one of said target genes:
   (a) that is greater than the second quartile of the expression levels of said target gene in a population of individuals who are suffering from said hematologic malignancy; or
   (b) that is greater than the second quartile of the expression levels of said target gene in a population of individuals who did not successfully respond to a treatment for said hematologic malignancy that used a CD123×CD3 bispecific molecule; or
   (c) that has a log₂-fold change of at least about 0.5 relative to the expression levels of said target gene in a population of individuals who did not successfully respond to a treatment for said hematologic malignancy that used a CD123×CD3 bispecific molecule; or
   (d) that is within at least the second quartile of the expression levels of said target gene in a population of individuals who successfully responded to a treatment for said hematologic malignancy that used a CD123×CD3 bispecific molecule.
E14. The method of any one of E2-E11, wherein said patient exhibits an expression level of at least one of said target genes:
   (a) that is greater than the third quartile of the expression levels of said target gene in a population of individuals who are suffering from said hematologic malignancy; or
   (b) that is greater than the third quartile of the expression levels of said target gene in a population of individuals who did not successfully respond to a treatment for said hematologic malignancy that used a CD123×CD3 bispecific molecule; or
   (c) that has a log₂-fold change of at least about 0.6 relative to the expression levels of said target gene in a population of individuals who did not successfully respond to a treatment for said hematologic malignancy that used a CD123×CD3 bispecific molecule.
E15. A method of treating a hematologic malignancy, wherein said method comprises:
   (a) employing the method of any one of E6-E14 to determine whether a patient would be a suitable responder to the use of a CD123×CD3 bispecific molecule to treat said hematologic malignancy;
   (b) administering a treatment dosage of said CD123×CD3 bispecific molecule to said patient if said patient is determined to be a suitable responder to such treatment;
   wherein said administration of said CD123×CD3 bispecific molecule stimulates the killing of cells of said hematologic malignancy in said patient.
E16. The method of E15, wherein said method additionally comprises evaluating the expression of said one or more target genes of said patient one or more times after the initiation of said treatment.
E17. A method of treating a hematologic malignancy, comprising:
   (a) administering an effective treatment dosage of a CD123×CD3 bispecific molecule;
   (b) determining the expression of one or more target genes in a cellular sample obtained from said patient at one or more time points following administration of said CD123×CD3 bispecific molecule relative to a corresponding baseline level of expression obtained prior to administration of said CD123×CD3 bispecific molecule;
   (c) determining whether the expression of said one or more target genes is increased relative to said corresponding baseline level of expression, wherein a determination of such increased gene expression identifies said patient as a suitable responder for treatment with a CD123×CD3 bispecific molecule; and
   (d) administering an adjusted or additional effective treatment dosage of said CD123×CD3 bispecific molecule to any such suitable responder patients,
   wherein said administration of CD123×CD3 bispecific molecule stimulates the killing of cells of said hematologic malignancy in said patient.
E18. The method of any one of E2-E17, wherein said cellular sample is a blood sample.
E19. The method of any one of E2-E17, wherein said cellular sample is a bone marrow sample.
E20. The method of any one of E2-E17, comprising detecting the expression level of said one or more target genes and/or said one or more reference genes in a sample of the patient's bone marrow.
E21. The method of any one of E2-E20, wherein said evaluation of expression or said determination of whether said patient would be a suitable responder to the use of a CD123×CD3 bispecific molecule to treat a hematologic malignancy is performed by:
   (a) determining the gene expression levels for each target gene in one or more cellular sample(s) using a gene expression platform; and
   (b) comparing said target gene expression levels to the expression levels of one or more reference genes.
E22. The method of any one of E2-E21, wherein said evaluation of expression or said determination of whether said patient would be a suitable responder to the use of a CD123×CD3 bispecific molecule to treat a hematologic malignancy is performed by:
   (a) measuring the raw RNA levels in for each target gene in one more cellular sample using a gene expression platform, wherein the gene expression platform comprises a reference gene set of housekeeping genes, and
   (b) assigning a relative expression value, for each of the measured raw RNA levels for the target genes using the measured RNA levels of the internal reference genes.
E23. The method of any one of E2-E22, wherein said one or more target genes comprise:
   (a) one or more of: CXCL9, CXCL10, CXCL11, and STAT1; and/or
   (b) one or more of: CCL5, CD27, CD274, CD276, CD8A, CMKLR1, CXCL9, CXCR6, HLA-DQA1, HLA-DRB1, HLA-E, IDO1, LAG3, NKG7, PDCD1LG2, PSMB10, STAT1, and TIGIT; and/or
   (c) one or more of: AREG, CSF3, CXCL1, CXCL2, CXCL3, CCL20, FOSL1, IER3 (NM_003897.4), IL6 and PTGS2; and/or
   (d) one or more of: CCL2, CCL3/L1, CCL4, CCL7 and CCL8; and/or
   (e) one or more of: MAGEA3/A6, MAGEA1, MAGEA12, MAGEA4, MAGEB2, MAGEC1 and MAGEC2; and/or
   (f) one or more of: APOL6, DTX3L, GBP1, IFI16, IFI27, IFI35, IFI6, IFIH1, IFIT1, IFIT2, IFIT3, IFITM1, IFITM2, IRF1, IRF9, ISG15, MX1, OAS1, OAS2, PARP9, PSMB9, STAT2, TMEM140 and TRIM21; and/or
   (g) one or more of: PSMB8, PSMB9 and PSMB10; and/or
   (h) IL-10; and or
   (i) CD274; and/or
   (j) PDCD1LG2.
E24. The method of E23, where said one or more target genes further comprises IFNG.
E25. The method of any one of E2-E24, wherein said one or more reference genes comprise one or more of: ABCF1, G6PD, NRDE2, OAZ1, POLR2A, SDHA, STK11IP, TBC1D10B, TBP, and UBB.
E26. The method of any one of E2-E25, wherein a gene signature score is determined for said one or more target genes.
E27. The method of E26, wherein said gene signature score is determined by a process comprising:
   (a) measuring the raw RNA levels for each target gene in one more cellular sample using a gene expression platform comprising a reference gene set of housekeeping genes,
   (b) normalizing each of the measured raw RNA levels to the geometric mean of said housekeeping genes, and optionally further normalizing each RNA value to a standard,
   (c) log transforming each normalized RNA value,
   (d) multiplying each log transformed RNA value by a corresponding weight factor to generate a weighted RNA value, and
   (e) adding the weighted RNA values, and optionally adding an adjustment factor constant, to generate a single gene signature score.
E28. The method of E26 or E27, wherein said gene signature score is determined using the target gene(s), the scoring weights and optionally the adjustment factors provided in Tables 6 and 12A-12G.
E29. The method of any one of E26-E28, wherein said gene signature score is a gene signature score determined for one or more of:
   (a) the IFN Gamma Signaling Signature;
   (b) the Tumor Inflammation Signature;
   (c) the Myeloid Inflammation Signature;
   (d) the Inflammatory Chemokine Signature;
   (e) the MAGEs Signature;
   (f) the IFN Downstream Signaling Signature;
   (g) the Immunoproteasome Signature;
   (h) the IL-10 Signature;
   (i) the CD274 Signature; and/or
   (j) the PDCD1LG2 Signature.
E30. The method of any one of E26-E29, wherein a patient gene signature score that:
   (a) is greater than the first quartile of scores for said gene signature calculated from the expression levels of one or more of said target genes in a population of individuals who are suffering from said hematologic malignancy; or
   (b) is greater than the first quartile of scores for said gene signature calculated from the expression levels of one or more of said target genes in a population of individuals who did not successfully respond to a treatment for said hematologic malignancy that used a CD123×CD3 bispecific molecule; or
   (c) has a log₂-fold change of at least about 0.4 relative to scores for said gene signature calculated from the expression levels of one or more of said target genes in a population of individuals who did not successfully respond to a treatment for said hematologic malignancy that used a CD123×CD3 bispecific molecule; or
   (d) is within at least the first quartile of the scores for said gene signature calculated from the expression levels of one or more of said target genes in a population of individuals who successfully responded to a treatment for said hematologic malignancy that used a CD123×CD3 bispecific molecule,
   is indicative of a more favorable patient response to treatment with said CD123×CD3 bispecific molecule.
E31. The method of any one of E26-E29, wherein a patient gene signature score that:
   (a) is greater than the second quartile for said gene signature calculated from the expression levels of one or more of said target genes in a population of individuals who are suffering from said hematologic malignancy; or
   (b) is greater than the second quartile for said gene signature calculated from the expression levels of one or more of said target genes in a population of individuals who did not successfully respond to a treatment for said hematologic malignancy that used a CD123×CD3 bispecific molecule; or
   (c) has a log₂-fold change of at least about 0.5 relative to scores for said gene signature calculated from the expression levels of one or more of said target genes in a population of individuals who did not successfully respond to a treatment for said hematologic malignancy that used a CD123×CD3 bispecific molecule; or
   (d) is within at least the second quartile of the scores for said gene signature calculated from the expression levels of one or more of said target genes in a population of individuals who successfully responded to a treatment for said hematologic malignancy that used a CD123×CD3 bispecific molecule,
   is indicative of a more favorable patient response to treatment with said CD123×CD3 bispecific molecule.
E32. The method of any one of E26-E29, wherein a patent gene signature score that:
   (a) is greater than the third quartile of scores for said gene signature calculated from the expression levels of one or more of said target genes in a population of individuals who are suffering from said hematologic malignancy; or
   (b) is greater than the third quartile of scores for said gene signature calculated from the expression levels of one or more of said target genes in a population of individuals who did not successfully respond to a treatment for said hematologic malignancy that used a CD123×CD3 bispecific molecule; or
   (c) has a log₂-fold change of at least about 0.6 relative to scores for said gene signature calculated from the expression levels of one or more of said target genes in a population of individuals who did not successfully respond to a treatment for said hematologic malignancy that used a CD123×CD3 bispecific molecule,
   is indicative of a more favorable patient response to treatment with said CD123×CD3 bispecific molecule.
E33. The method of any one of E28-E29, wherein:
   (a) said gene signature is the IFN Gamma Signaling Signature, and a patient gene signature score of at least about 2.5 is indicative of a more favorable patient response to treatment with said CD123×CD3 bispecific molecule, and/or
   (b) said gene signature is the Tumor Inflammation Signature, and a patient gene signature score of at least about 5.5 is indicative of a more favorable patient response to treatment with said CD123×CD3 bispecific molecule; and/or
   (c) said gene signature is the IFN Downstream Signaling Signature, and a patient gene signature score of at least about 4.5 is indicative of a more favorable patient response to treatment with said CD123×CD3 bispecific molecule.
E34. The method of any one of E28-E29, wherein said gene signature is the IFN Gamma Signaling Signature, the Tumor Inflammation Signature, or the IFN Downstream Signaling Signature, and a patient gene signature score that:
   (a) is greater than the first quartile of scores of said gene signature calculated from the expression levels of one or more of said target genes in a population of individuals who are suffering from said hematologic malignancy; or
   (b) is greater than the first quartile of scores for said gene signature calculated from the expression levels of one or more of said target genes in a population of individuals who did not successfully respond to a treatment for said hematologic malignancy that used a CD123×CD3 bispecific molecule; or
   (c) has a log₂-fold change of at least about 0.4 relative to scores for said gene signature calculated from the expression levels of one or more of said target genes in a population of individuals who did not successfully respond to a treatment for said hematologic malignancy that used a CD123×CD3 bispecific molecule; or
   (d) is within at least the first quartile of the scores for said gene signature calculated from the expression levels of one or more of said target genes in a population of individuals who successfully responded to a treatment for said hematologic malignancy that used a CD123×CD3 bispecific molecule,
   is indicative of a more favorable patient response to treatment with said CD123×CD3 bispecific molecule.
E35. The method of any one of E28-E29, wherein said gene signature is the IFN Gamma Signaling Signature, the Tumor Inflammation Signature, or the IFN Downstream Signaling Signature, and a patient gene signature score that:
   (a) is greater than the second quartile of scores of said gene signature calculated from the expression levels of one or more of said target genes in a population of individuals who are suffering from said hematologic malignancy; or
   (b) is greater than the second quartile of scores for said gene signature calculated from the expression levels of one or more of said target genes in a population of individuals who did not successfully respond to a treatment for said hematologic malignancy that used a CD123×CD3 bispecific molecule; or
   (c) has a log₂-fold change of at least about 0.5 relative to scores for said gene signature calculated from the expression levels of one or more of said target genes in a population of individuals who did not successfully respond to a treatment for said hematologic malignancy that used a CD123×CD3 bispecific molecule; or
   (d) is within at least the second quartile of the scores for said gene signature calculated from the expression levels of one or more of said target genes in a population of individuals who successfully responded to a treatment for said hematologic malignancy that used a CD123×CD3 bispecific molecule,
   is indicative of a more favorable patient response to treatment with said CD123×CD3 bispecific molecule.
E36. The method of any one of E28-E29, wherein said gene signature is the IFN Gamma Signaling Signature, the Tumor Inflammation Signature, or the IFN Downstream Signaling Signature, and a patient gene signature score that:
   (a) is greater than the third quartile of scores of said gene signature calculated from the expression levels of one or more of said target genes in a population of individuals who are suffering from said hematologic malignancy; or
   (b) is greater than the third quartile of scores for said gene signature calculated from the expression levels of one or more of said target genes in a population of individuals who did not successfully respond to a treatment for said hematologic malignancy that used a CD123×CD3 bispecific molecule; or
   (c) has a log₂-fold change of at least about 0.6 relative to scores for said gene signature calculated from the expression levels of one or more of said target genes in a population of individuals who did not successfully respond to a treatment for said hematologic malignancy that used a CD123×CD3 bispecific molecule,
   is indicative of a more favorable patient response to treatment with said CD123×CD3 bispecific molecule.
E37. The method of E29, wherein an IFN Dominant Module score is determined, and wherein a patient IFN Dominant Module score of at least about 25 is indicative of a more favorable patient response to treatment with said CD123×CD3 bispecific molecule.
E38. The method of E29, wherein an IFN Dominant Module score is determined, and wherein a patient IFN Dominant Module score that:
   (a) is greater than the first quartile of scores of said IFN Dominant Module calculated from the expression levels of one or more of said target genes in a population of individuals who are suffering from said hematologic malignancy; or
   (b) is greater than the first quartile of scores for IFN Dominant Module calculated from the expression levels of one or more of said target genes in a population of individuals who did not successfully respond to a treatment for said hematologic malignancy that used a CD123×CD3 bispecific molecule; or
   (c) is within at least the first quartile of the scores for said IFN Dominant Module calculated from the expression levels of one or more of said target genes in a population of individuals who successfully responded to a treatment for said hematologic malignancy that used a CD123×CD3 bispecific molecule,
   is indicative of a more favorable patient response to treatment with said CD123×CD3 bispecific molecule.
E39. The method of E29, wherein an IFN Dominant Module score is determined, and wherein a patient IFN Dominant Module score that:
   (a) is greater than the second quartile of scores of said IFN Dominant Module calculated from the expression levels of one or more of said target genes in a population of individuals who are suffering from said hematologic malignancy; or
   (b) is greater than the second quartile of scores for IFN Dominant Module calculated from the expression levels of one or more of said target genes in a population of individuals who did not successfully respond to a treatment for said hematologic malignancy that used a CD123×CD3 bispecific molecule; or
   (c) is within at least the second quartile of the scores for said IFN Dominant Module calculated from the expression levels of one or more of said target genes in a population of individuals who successfully responded to a treatment for said hematologic malignancy that used a CD123×CD3 bispecific molecule, is indicative of a more favorable patient response to treatment with said CD123×CD3 bispecific molecule.
E40. The method of any one of E26-E39, wherein a patient that exhibits a gene expression signature that is characteristic of an immune-enriched and IFN gamma-dominant tumor microenvironment is indicative of a more favorable patient response to treatment with said CD123×CD3 bispecific molecule.
E41. The method of any one of E1-E40, wherein said CD123×CD3 bispecific molecule is a bispecific antibody or bispecific molecule comprising an scFv.
E42. The method of E41, wherein said CD123×CD3 bispecific molecule is JNJ-63709178, XmAb14045 or APVO436.
E43. The method of any one of E1-E40, wherein said CD123×CD3 bispecific molecule is a covalently bonded bispecific diabody having two, three, or four polypeptide chains.
E44. The method of E43, wherein said CD123×CD3 bispecific molecule is a diabody that comprises:
   (a) a first polypeptide chain having the amino acid sequence of SEQ ID NO:21; and
   (b) a second polypeptide chain having the amino acid sequence of SEQ ID NO:23; and
   wherein the first and said second polypeptide chains are covalently bonded to one another by a disulfide bond.
E45. The method of any one of E1-E44, wherein said hematologic malignancy of said patient is selected from the group consisting of: acute myeloid leukemia (AML), chronic myelogenous leukemia (CIVIL), blastic crisis of CML, Abelson oncogene-associated with CIVIL (Bcr-ABL translocation), myelodysplastic syndrome (MDS), acute B lymphoblastic leukemia (B-ALL), acute T lymphoblastic leukemia (T-ALL), chronic lymphocytic leukemia (CLL), Richter's syndrome, Richter's transformation of CLL, hairy cell leukemia (HCL), blastic plasmacytoid dendritic cell neoplasm (BPDCN), non-Hodgkin's lymphoma (NHL), including mantle cell lymphoma (MCL) and small lymphocytic lymphoma (SLL), Hodgkin's lymphoma, systemic mastocytosis, and Burkitt's lymphoma.
E46. The method of E45, wherein said hematologic malignancy of said patient is AML.
E47. The method of E45, wherein said hematologic malignancy of said patient is MDS.
E48. The method of E45, wherein said hematologic malignancy of said patient is BPDCN.
E49. The method of E45, wherein said hematologic malignancy of said patient is T-ALL.
E50 The method of any one of E2-E49, wherein said hematologic malignancy of said patient is refractory to chemotherapy.
E51. The method of E1 or E50, wherein said hematologic malignancy of said patient is refractory to cytarabine/anthracycline-based cytotoxic chemotherapy.
E52. The method of E1 or E50, wherein said hematologic malignancy of said patient is refractory to hypomethylating agent chemotherapy.
E53. The method of any one of E2-E52, further comprising determining the level expression of CD123 of blast cells (cancer cells) as compared to a corresponding baseline level CD123 expressed by normal PBMCs.
E54. The method of E56, wherein said level of expression is measured by cell surface expression of CD123.
E55. The method of E54, wherein said surface expression of CD123 is increased by at least about 20% relative to a baseline level of expression.
E56. The method of E55, wherein said increase in CD123 expression renders the patient more responsive to treatment with said CD123×CD3 bispecific molecule.
E57. The method of any one of E1-E4, or E6-E56, wherein said treatment dosage of said CD123×CD3 bispecific molecule includes at least one dose selected from the group consisting of 30, 100, 300, and 500 ng/kg patient weight/day.
E58. The method of E57, wherein said treatment dosage includes a dose of 30 ng/kg/day.
E59. The method of E57, wherein said treatment dosage includes a dose of 100 ng/kg patient weight/day.
E60. The method of E57, wherein said treatment dosage includes a dose of 300 ng/kg patient weight/day.
E61. The method of E57, wherein said treatment dosage includes a dose of 500 ng/kg patient weight/day.
E62. The method of any one of E1-E4, or E6-E561, wherein said treatment dosage is administered by continuous infusion.
E63. The method of any one of E1-E62, wherein said patient is a human patient.

### EXAMPLES

Having now generally described the invention, the same will be more readily understood through reference to the following examples, which are provided by way of illustration and are not intended to be limiting of the present invention unless specified.

### Example 1

### Gene Expression Signatures of Patient Populations Particularly Suitable for Treatment with a CD123×CD3 Bispecific Binding Molecule of the Invention

In order to demonstrate a correlation between the expression patterns of the genes of patients having a hematologic malignancy, particularly AML, and the favorable outcome of CD123×CD3 bispecific binding molecule therapy, RNA was isolated from 78 bone marrow (“BM”) samples obtained from patients with individual patient consent (36 at baseline, 27 after a first treatment cycle and 15 after a second treatment cycle) from 40 patients with relapsed or refractory AML enrolled in a phase 1/2 clinical trial of flotetuzumab (NCT #02152956, an exemplary CD123×CD3 bispecific binding molecule). Gene expression was evaluated using the nCounter™ system (NanoString Technologies, Inc), which enables direct multiplexed mRNA quantification of low-abundance transcripts in a single reaction with high sensitivity and linearity (Vadakekolathu, J., et al. (2017) “Immune gene Expression Profiling In Children And Adults With Acute Myeloid Leukemia Identifies Distinct Phenotypic Patterns,” Blood 130:3942-3942; Payton, J. E., et al. (2009). “High throughput digital quantification of mRNA abundance in primary human acute myeloid leukemia samples,” J Clin Invest 119:1714-1726). Baseline bone marrow samples from 36 patients were included in the analysis, of which 35 patients were treated at a dose of ≥500 ng/kg/day. The NanoString PanCancer IO 360™ assay (NanoString Technologies, Inc.) compared the expression profiles of 750 genes that cover the key pathways at the interface of the tumor, tumor microenvironment, and immune response, including the levels of 14 immune cell types and 32 immuno-oncology signatures. The NanoString PanCancer IO 360™ assay also compared the expression profiles of control and internal reference genes for data normalization as provided below.

The expression profile included gene signatures of the following pathways or cells: Proliferation, JAKSTAT loss, endothelial cells, B7-H3, APM loss, glycolytic activity, mast cells, cytotoxicity, cytotoxic cells, CD8 T cells, lymphoid cells, T cells, Treg cells, CTLA4, TIS, Th1 cells, TIGIT, NK CD56^{dim} cells, NK cells, apoptosis, hypoxia, ARG1, IL-10, IFN gamma, macrophages, myeloid cells, neutrophils, PD-L2, stroma, dendritic cells (DC), MAGEs, IDO1, B cells, PD-1, NOS2, inflammatory chemokines, PD-L1, CD45, exhausted CD8 T cells, immunoproteasome, APM, IFN downstream regulated genes, myeloid inflammatory genes, MHC2 genes, TGF beta, MMR loss.

All IO 360 Gene Signature analysis was performed using the nCounter™ system (NanoString Technologies, Inc.) with the IO 360 Report module essentially as described below).

The Interferon (IFN) Gamma Signaling Signature genes (including a representative, non-limiting NCBI accession number for each gene), and weight factors are shown in Table 6 below.

TABLE 6
The Interferon (IFN) Gamma Signaling Signature Genes

| Signature | Gene | NCBI Accession No. | Weight |
|---|---|---|---|
| IFN gamma | STAT1 | NM_007315.2 | 0.261104 |
| IFN gamma | CXCL9 | NM_002416.1 | 0.188978 |
| IFN gamma | CXCL10 | NM_001565.3 | 0.308838 |
| IFN gamma | CXCL11 | NM_005409.4 | 0.24108 |

To calculate the IFN Gamma Signaling Signature score, the following steps are performed:
Raw data counts for each gene are normalized to the geometric mean of 10 housekeeping (HK) genes (ABCF1, NRDE2, G6PD, OAZ1, POLR2A, SDHA, STK11IP, TBC1D10B, TBP, UBB) for each sample.
HK normalized data is then normalized to IO 360 panel standards, in this case those run on the same cartridges as the cohort samples.
Each normalized gene count is then log transformed.
Once normalized and log transformed, each gene is multiplied to the weight in Table 6.
Each of these weighted counts is summed to generate a single score. An adjustment factor, that is a constant is added to the final calculated score, for the IFN Gamma Signaling Signature the adjustment factor is 6.457026. The adjustment factor was derived from the lowest observed score (from TCGA and cell line analysis), in order for the score range to be above 0.

Generally, the possible range of IFN Gamma Signaling Signature scores is 0 to 10. For this first cohort the range is 1 to 5. The score is calculated for each baseline (screen day −14) sample.

The genes and weight factor for additional signatures examined are provided below.

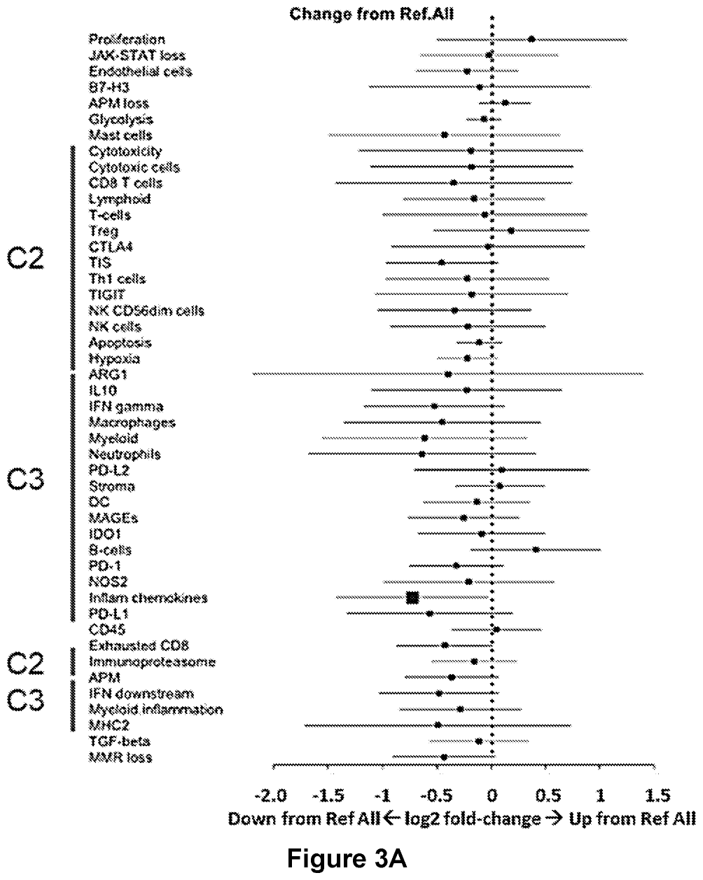

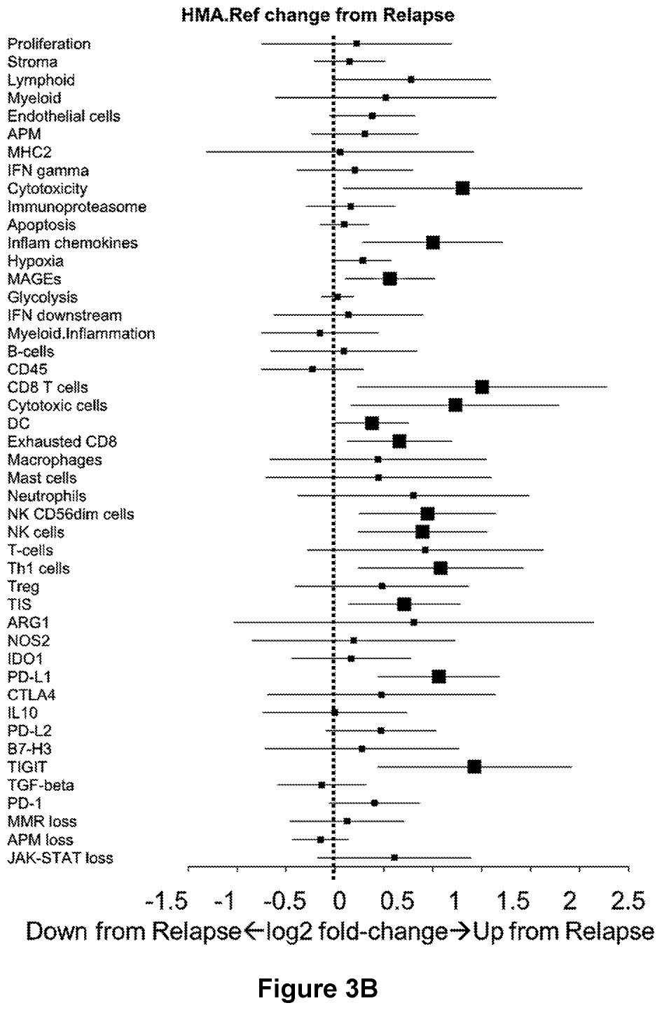

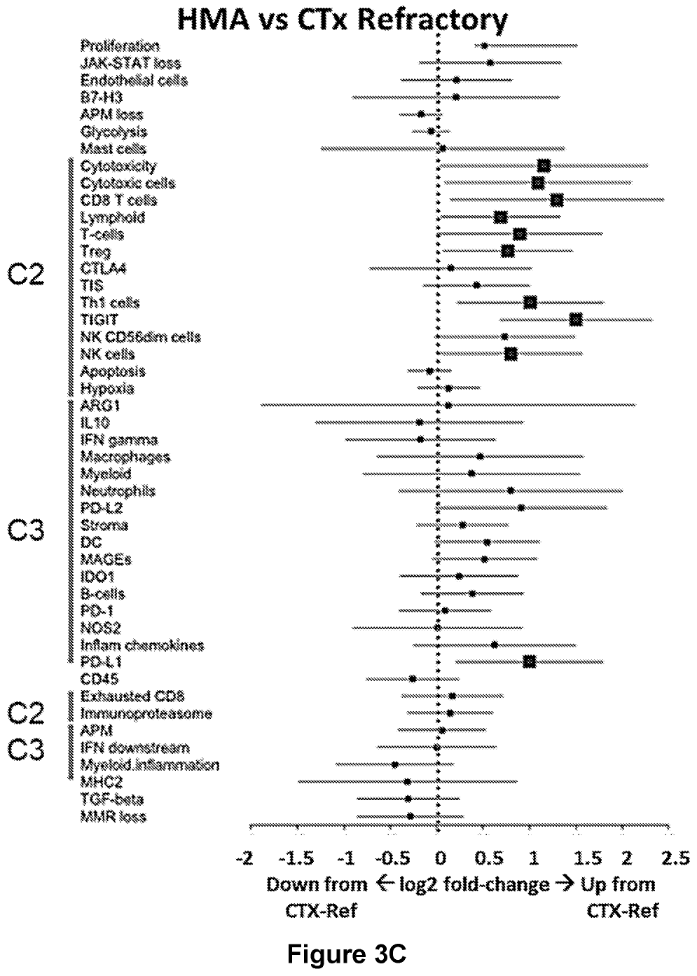

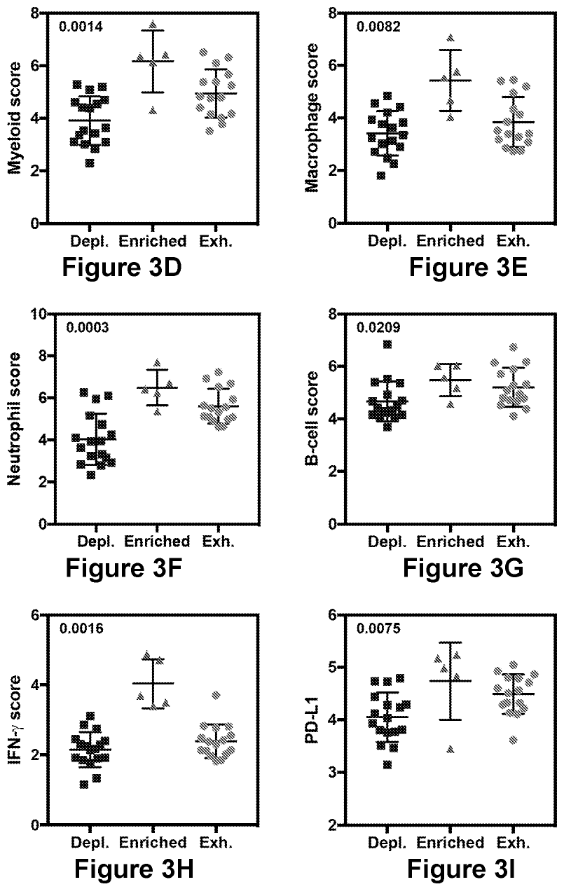

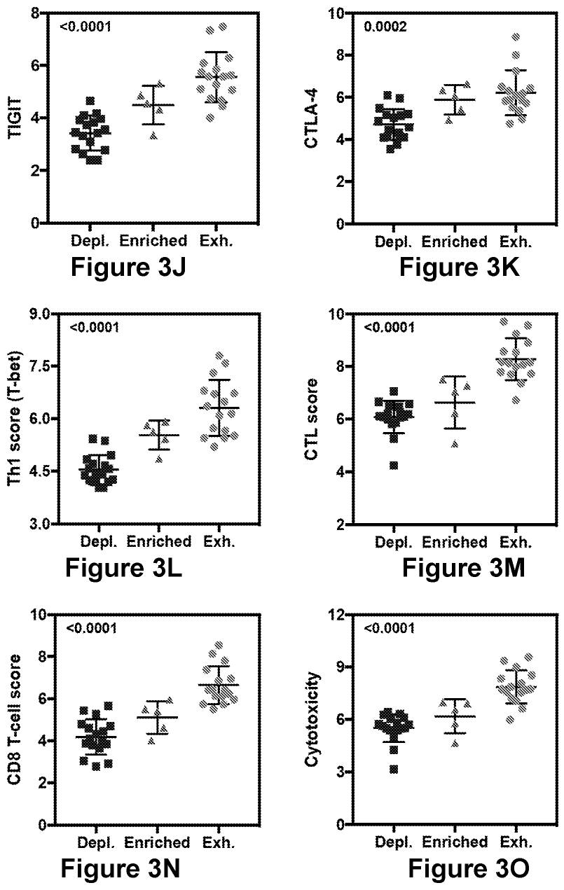

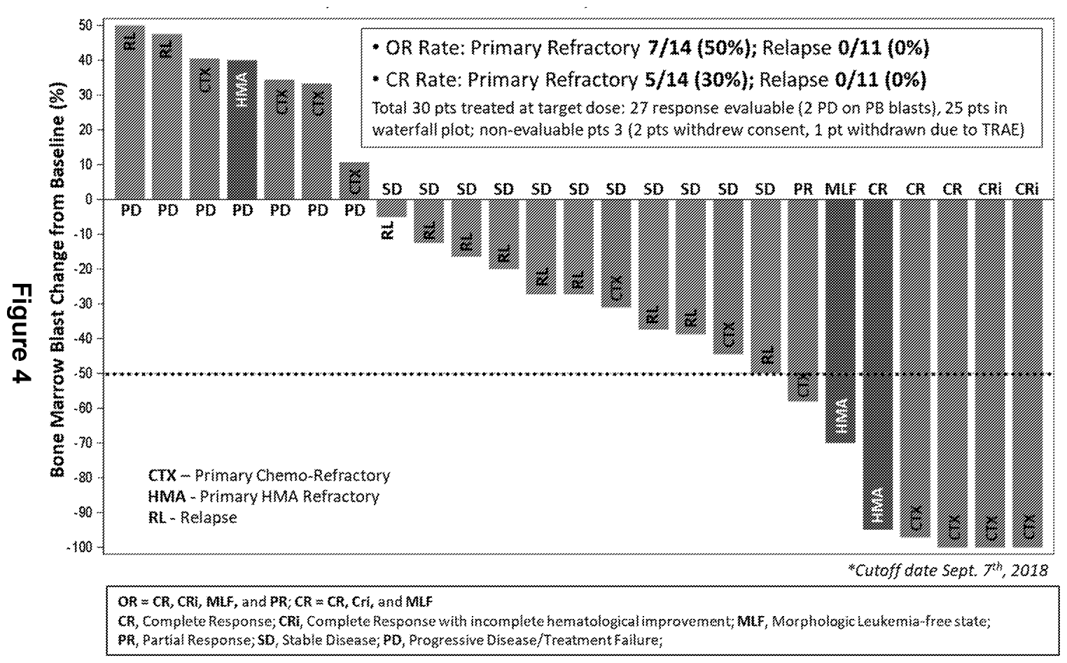

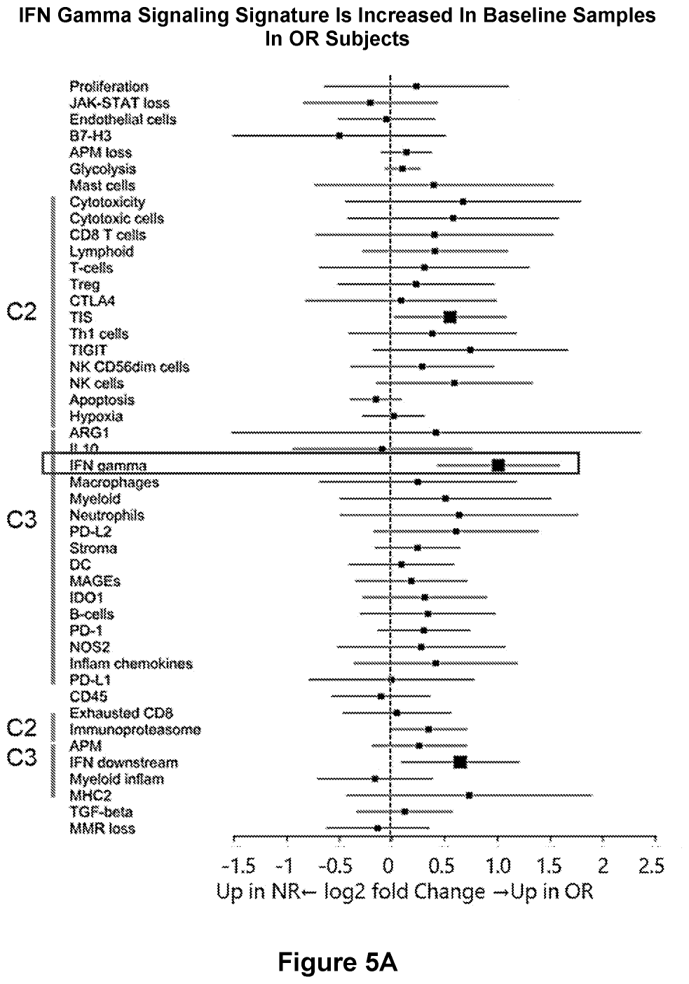

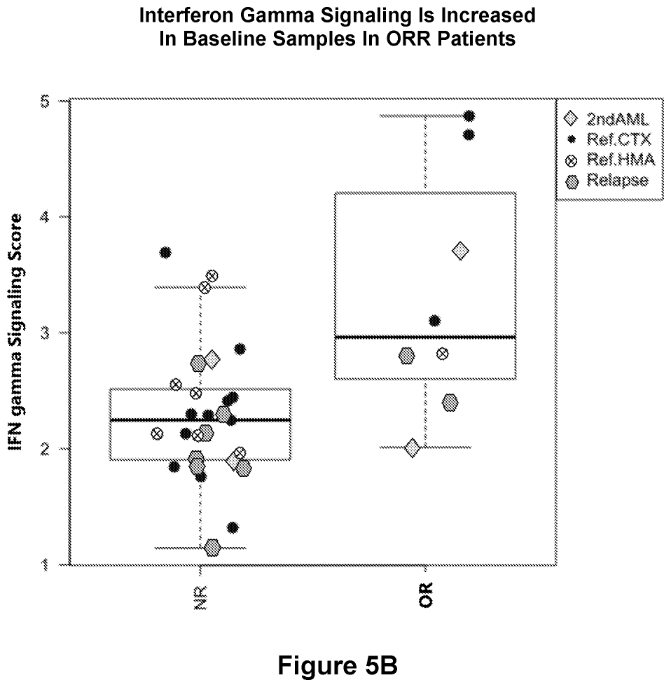

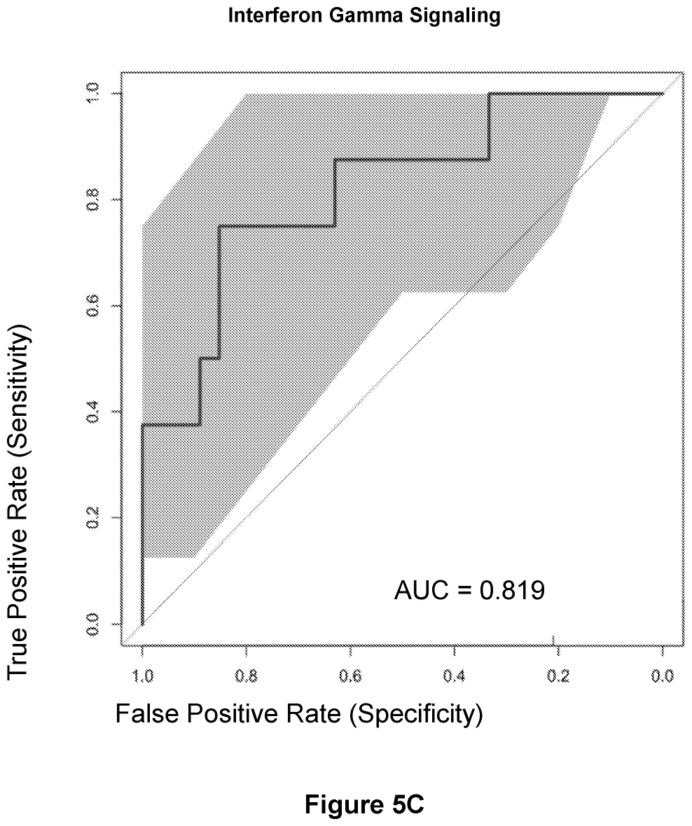

Several analyses were performed comparing IFN Gamma Signaling Signature scores (and all IO 360 signature scores) for this cohort as detailed below. Fold-change differences between different patient groups are provided as Forest plots where box size represents significance and each line represents the confidence intervals (see, e.g., FIGS. 3A-3C, and FIG. 5A). Distribution of IFN Gamma Signaling Signature scores between patent groups are provided as box plots (see, e.g., FIG. 5B).

Baseline expression of the profiled genes was correlated with whether the patient had a refractory response to conventional chemotherapy (i.e., patient refractory response to a regimen of treatment with cytarabine given in conjunction with daunorubicin (e.g., 7+3 induction therapy, (abbreviated as Ref CTX or CTX-refractory)) or patient refractory response to a regimen of treatment with the hypomethylating agents (e.g., decitabine and azacitidine, (abbreviated as Ref HMA or HMA-refractory)) or to patient relapse (Relapse). Patients having secondary AML (i.e., AML evolving from myelodysplasia or as product of previous chemotherapy) are including with the HMA-refractory group for these analysis. The data was also correlated to the patients' responses to CD123×CD3 bispecific binding molecule therapy with flotetuzumab. FIG. 4 provides a waterfall plot of 25 evaluable patients treated at the target dose. Such responses were scored as being either an objective response (OR) or as non-responding (NR). In addition to patients exhibiting a complete response, (CR), OR included all patients that exhibited a molecular complete response (mCR), a complete response with incomplete hematological improvement (CRi), a morphologic leukemia-free state (MLF), and a partial response (PR). In addition to non-responding patients, NR included all patients that exhibited progressive disease/treatment failure (PD), and stable disease (SD).

FIG. 2 provides an unsupervised hierarchical clustering the 46 IO 360 signatures or cell types generated from the results. The results show the baseline levels of expression of the 36 bone marrow samples (each in a separate column) relative to the gene signature evaluated (each in a separate row). Each IO 360 signature score was rescaled within the score for this cohort to a −3 to +3 scale to facilitate comparison across signatures.

Gene expression analysis of the BM samples at baseline stratifies AML patients into 3 clusters within an immunological continuum: immune-depleted, immune-exhausted and immune-enriched (FIG. 2), patients with primary-refractory disease (Relapse; refractory to ≥2 induction attempts, first CR of <6 months, or failure after ≥4 cycles of hypomethylating agents, HMA) showed prevalently an immune-infiltrated tumor microenvironment (TME) phenotype, which included higher inflammatory chemokine scores compared with relapse patients (3.27±0.22 vs 2.46±0.07, p=0.026). Within this group, chemotherapy-refractory and HMA-refractory patients further stratify into immune-enriched and immune-exhausted phenotypes, respectively. The gene signatures associated with immune-exhausted and immune enriched phenotypes are listed in Table 7 below and indicted on the forest plots shown in FIGS. 3A, 3B, and 4A.

TABLE 7
List of Gene Signatures
Immune Exhausted Cluster 2(C2)

| | |
|---|---|
| Cytotoxicity | TH1 |
| Cytotoxic cells | TIGIT |
| CD8 T cells | NK/NK^{dim} |
| Lymphoid | Apoptosis |
| T cells | Hypoxia |
| Treg | ARG1 |
| CTLA4 | Exhausted CD8 |
| TIS | Immunoproteasome |

Immune Enriched Cluster 3 (C3)

| | |
|---|---|
| IL10 | B cells |
| IFN gamma | PD1 |
| Macrophages | NOS2 |
| Myeloid | Inflammatory chemokines |
| Neutrophils | PDL1 |
| PDL2 | APM |
| Stroma | IFN downstream |
| DC | Myeloid inflammation |
| MAGES | MHC2 |
| IDO1 | |

Forest plots of the base-line fold change differences in a number of gene signatures between all refractory and relapsed patients (FIG. 3A) between HMA-refractory and relapsed patients (FIG. 3B), between HMA-refractory and CTX-refractory patients (FIG. 3C) indicate that HMA-Refractory patients exhibit a more senescent phenotype. Specifically, HMA-refractory patients displayed features of immune exhaustion and adaptive immune resistance, including upregulation of TIGIT (5.55±0.34 vs 3.85±0.24, p=0.006), PD-L1 (3.55±0.18 vs 2.4±0.29, p=0.009) and Treg cells (4.87±0.23 vs 3.69±0.19, p=0.0009) together with a trend toward increasingly exhausted CD8 T cells (CD244, EOMES, LAG3 and PTGER4) compared to CTX-refractory patients (FIGS. 3A-3C). Plotted in FIGS. 3D-3O are several gene signature scores associated with the Immune Enriched (Cluster 2, FIG. 3D-3I) or the Immune Exhausted (Cluster 3, FIG. 3J-3O) profiles. The Myeloid (FIG. 3D), Macrophage (FIG. 3E), Neutrophil (FIG. 3F), B-cell (FIG. 3G), IFN gamma (FIG. 3H), PD-L1 (FIG. 3I), TIGIT (FIG. 3J), CTLA-4 (FIG. 3K), Th1 (FIG. 3L), CTL (FIG. 3M), CD8 T cell (FIG. 3N), and Cytotoxicity (FIG. 3O), gene signature scores are plotted for each cluster (Immune Depleted (Depl.), Immune Enriched (Enriched), and Immune Exhausted (Exh.) and the p values (Kruskal-Wallis) are reported.

Comparative analysis of the IFN Gamma Signaling Signature score was done between OR patients (including all patients that exhibited CR, Complete Response; mCR, molecular CR; CRi, Complete Response with incomplete hematological improvement; MLF, Morphologic Leukemia-free state; or PR, Partial Response)) and NR patients (including all patients exhibiting SD, Stable Disease; or PD, Progressive Disease/Treatment Failure). FIG. 4 shows the change (relative to baseline) in blast cells present in bone marrow samples from 25 patients (categorized as being either relapse (RL) patients or patients that were Chemo-Refractory (CTX) or HMA-Refractory (HMA)) as a measure of their response to the CD123×CD3 bispecific binding molecule flotetuzumab at the target dose of 500 ng/kg/day. The objective response (OR) rate to the therapy for Primary Refractory patients was 50% ( 7/14). The complete response (CR) rate for Primary Refractory patients was 35.7% ( 5/14).

FIG. 5A presents a forest plot of the baseline fold change differences between OR patients and NR patients (including PD, SD, TF, NE) showing that expression of the IFN Gamma Signaling Signature was increased in baseline samples in OR patients (boxed in FIG. 5A, showing change from NR). In addition, the TIS and IFN Downstream Signatures were substantially increased in OR patients. FIG. 5B shows that the distribution of IFN Gamma Signaling Signature scores is increased in OR patients. In particular, responders to flotetuzumab showed significantly higher IFN Gamma Signaling Signature scores at baseline compared to non-responders (3.31±0.32 vs 2.27±0.11, p=0.0005). The sensitivity and specificity of the IFN Gamma Signaling Signature score was measured to predict response diagnostic capability. Bootstrapping over all samples is performed using different threshold cutoffs for the range of data in this cohort. The confidence intervals (CIs) of the thresholds or the sensitivity and specificity values are computed with bootstrap resampling and the averaging methods. In all bootstrap CIs, patients are resampled and the modified curve is built before the statistics of interest is computed. As in the bootstrap comparison test, the resampling is done in a stratified manner by default. The area under receiver operating characteristic (abbreviated herein as ROC) curves showing the predictive performance of the IFN Gamma Signaling Signature score with an area under curve (AUC)=0.819 are plotted in FIG. 5C. This plot shows the True Positive Rates (TPRs) and False Positive Rates (FPRs) that are achieved using optimal score cutoff for this cohort for calling a sample high or low for IFN Gamma Signaling Signature score. A signature with no predictive power will have an ROC curve along the diagonal, and a perfectly predictive signature will have a curve that reaches the top left corner. The shaded area surrounding the line indicates confidence intervals. These data are also consistent with the greater frequency of responders in primary refractory patients, which generally exhibited a higher INF Gamma Signaling Signature, compared to relapse patients. Accordingly, baseline IFN Gamma Signaling Signature scores show strong correlation with patient response to CD123×CD3 bispecific binding molecule therapy (AUC for flotetuzumab treated patients=0.919; FIG. 5C). Comparisons of immune signatures at baseline and response rates between the Clusters are summarized in Table 8 (Cluster Immune-depleted (Cluster 1) and Immune-infiltrated (Clusters 2-3)) and Table 9 (Immune-exhausted (Cluster 2) and Immune-enriched (Cluster 3)).

TABLE 8
Cluster Immune-depleted and Immune-infitrated

| | Immune-depleted | Immune-infiltrated |
|---|---|---|
| | (n = 17) | (n = 21) |
| Anti-leukemic activity | 5.9% (1/16) | 33.3% (6/18) |
| | 1 CRi | 3 CR, 2 OB, 1 PR |
| No response | 14 | 12 |
| N.A.* | 1 | 3 |
| ELN cytogenic risk | Favorable (n = 2) | Favorable (n = 5) |
| (at time of initial | Intermediate (n = 3) | Intermediate (n = 9) |
| diagnosis) | Adverse (n = 8) | Adverse (n = 5) |
| | N.A. (n = 4) | N.A. (n = 2) |

*Response data available in 35/38 patients

TABLE 9
Immune-exhausted and Immune-enriched

| | Immune-enriched | Immune-exhausted |
|---|---|---|
| | (n = 5) | (n = 16) |
| Anti-leukemic activity | 40.0% (2/5) | 25% (4/16) |
| | 1 CR, 1 OB | 2 CR, 1 OB, 1 PR |
| No response | 3 | 10 |
| N.A.* | — | 2 |
| Previous HMA treatment | 40% (2/5) | 62.5% (10/16) |
| ELN cytogenic risk | Favorable (n = 1) | Favorable (n = 4) |
| (at time of initial | Intermediate (n = 0) | Intermediate (n = 9) |
| diagnosis) | Adverse (n = 4) | Adverse (n = 1) |
| | | N.A. (n = 2) |

*Response data available in 35/38 patients

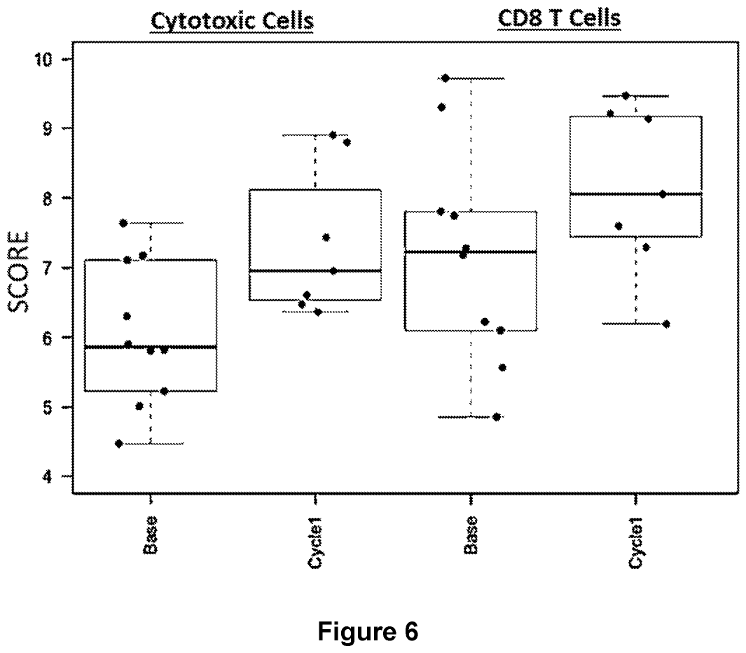

The gene expression signatures of a panel of genes associated with stimulation of Cytotoxic cells, or with CD8+ T cells, were examined in RNA from bone marrow samples pre-treatment (“Base”) and from bone marrow samples after a first cycle of treatment with flotetuzumab (“Cycle 1”). The results of this investigation are shown in FIG. 6. The results demonstrate that treatment with flotetuzumab was able to stimulate immune cells in the tumor microenvironment. Furthermore, comparison of post-cycle 1 BM samples to baseline samples showed treatment with flotetuzumab led to increased immune cell infiltrate and immune activation scores, as reflected by a higher Tumor Inflammation Signature; (6.49±0.20 vs 5.93±0.12, p=0.015) together with enhanced immunoproteasome (5.72±0.07 vs 5.23±0.10, p=0.0002) and IFN Gamma Signaling Signature (3.38±0.23 vs 2.53±0.14, p=0.0015) scores. Flotetuzumab-induced tumor microenvironment (TME) gene activation was therefore indicative of an immune-enrichment signature rather than an immune-exhaustion signature.

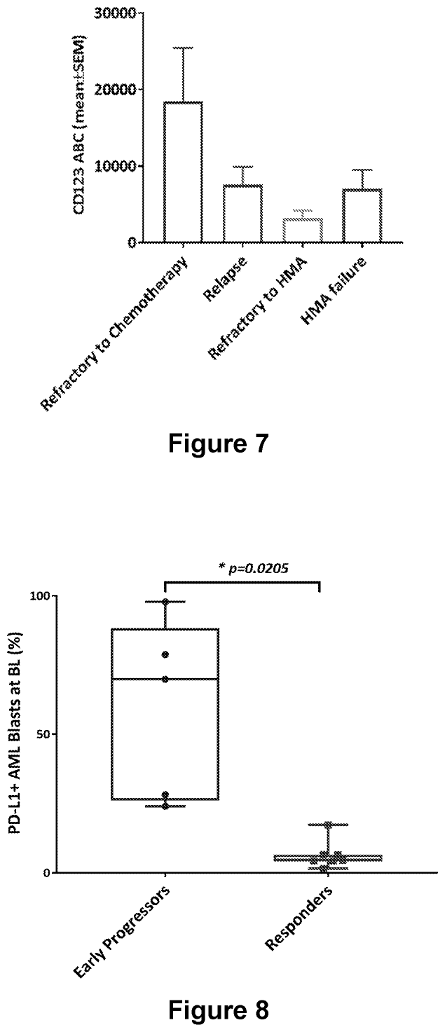

As shown in FIG. 7, the flotetuzumab responsive population—and in particular those patients previously refractory to chemotherapy—exhibited higher expression of CD123.

AML blast samples collected during screening were analyzed for PD-L1 expression by flow cytometry. As shown in FIG. 8, patients that progressed early (<15 days) on flotetuzumab treatment had higher baseline levels of PD-L1 on AML cells than other patients, and had evidence of response (SD, OB, PR, CR). The results of this investigation indicate that PD-L1 expression is associated with decreased activity in vivo and support the combinatorial use of a PD-1/PD-L1 antagonist in combination with a CD123×CD3 bispecific binding molecule therapy (see, e.g., WO 2017/214092).

Together these data indicate that the IFN Gamma Signaling Signature at baseline correlates with response to CD123×CD3 bispecific binding molecule therapy. Most patients showing evidence of anti-leukemic activity to CD123×CD3 bispecific binding molecule therapy (6/7; 86%) has high immune infiltration in the bone marrow, with the most sensitive population being the immune-enriched. In addition, patients previously-treated with HMA showed an immune-enriched but exhausted tumor microenvironment (e.g., bone marrow), with increased checkpoint expression, suggesting potential benefit from CD123×CD3 bispecific binding molecule therapy in combination with immune checkpoint blockade. Without being bound by any particular theory, CD123×CD3 bispecific binding molecule therapy may invigorate an immune exhausted tumor microenvironment as noted by 25% anti-leukemic activity in this population. In particular, treatment with the CD123×CD3 bispecific binding molecule, DART-A, was seen to enhance immune activation, antigen processing/presenting and IFN Gamma Signaling Signatures scores.

### Example 2

### Gene Expression Signatures of Relapsed and Chemotherapy-Refractory Patient Populations

Additional analysis was performed to further explore the correlation between higher expression of gene signatures, including but not limited to IFN Gamma Signaling Signature, TIS, and Interferon Downstream Signature, in immune-infiltrated AML cases, and benefit from treatment with bispecific immunotherapy agents targeting CD123×CD3, such as flotetuzumab. This analysis focused on the gene signatures and combinations of signatures (obtained using the NanoString PanCancer IO 360™ assay essentially as described below) from 30 chemotherapy-refractory (refractory to ≥2 induction attempts, first complete response of <6 months) or relapsed AML patients enrolled in the CP-MGD006-01 clinical trial (NCT #02152956). This analysis excluded samples from HMA-refractory patients and included additional samples from relapsed and chemotherapy-refractive patients not previously analyzed.

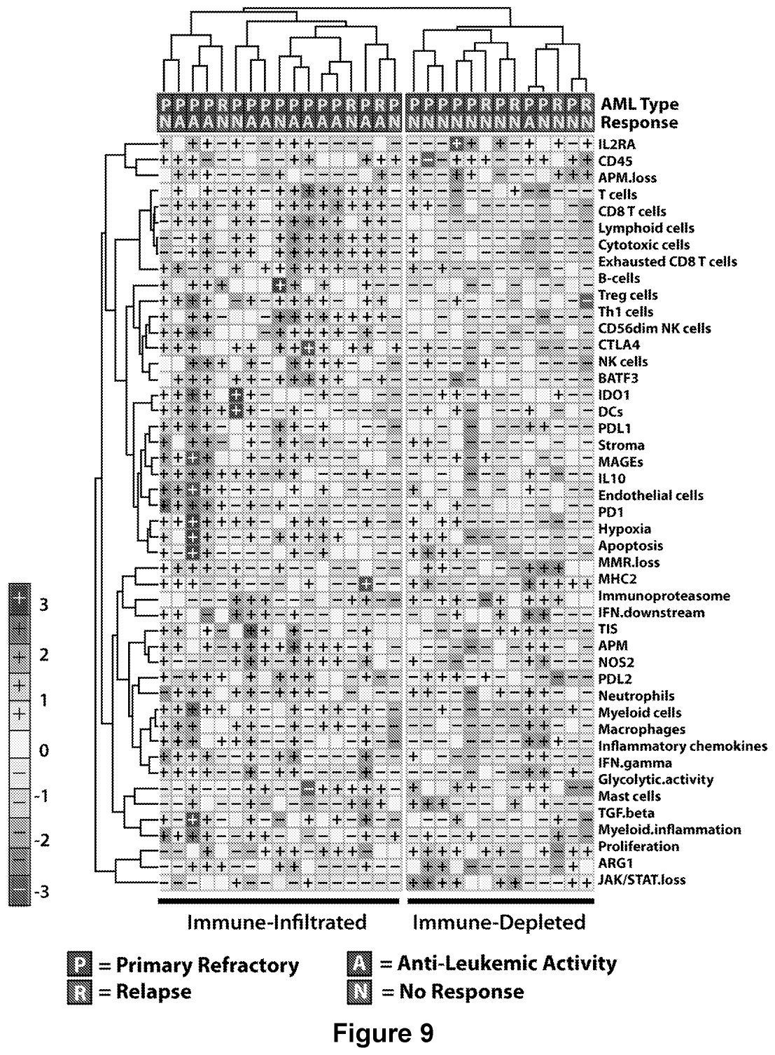

This analysis stratified relapsed and refractory AML patient BM samples at baseline into two immune subtypes, which will be herein termed immune-infiltrated and immune-depleted (FIG. 9) by aggregating the scores of three signature modules: IFN-Dominant, Adaptive and Myeloid. The gene signatures associated with the three signature modules are listed in Table 10 below. The module score is the sum of the individual gene signature scores in each sample (each gene signature score was calculated as provided above).

TABLE 10
List of Gene Signatures In Modules
IFN Dominant Module

| | | |
|---|---|---|
| Myeloid Inflammation | Inflammatory Chemokines | MAGES |
| IL10† | IFN Gamma Signaling | IFN Downstream |
| PDL1† | Immunoproteasome | PDL2† |

Adaptive

| | | |
|---|---|---|
| B cells | Exhausted CD8 | Cytotoxicity |
| Cytotoxic Cells | TBX21 (aka TH1) | T Cells |
| NK cells | CD8 T cells | TIGIT† |
| Lymphoid | TIS | FoxP3† |
| CTLA4† | PD1† | |

Myeloid Module

| | | |
|---|---|---|
| Myeloid | Macrophages | Neutrophils |
| DC | | |

†single-gene signatures

FIG. 9 provides an unsupervised hierarchical clustering (Euclidean distance, complete linkage) of immune and biological activity signatures in the bone marrow (BM) microenvironment of patients with relapsed/refractory AML prior to receiving flotetuzumab immunotherapy in the CP-MGD006-01 clinical trial (NCT #02152956). Responders were individuals exhibiting an anti-leukemic response defined as either complete remission (CR), CR with incomplete hematologic recovery (CRi), CR with partial hematologic recovery (CRh) partial remission (PR) or “other benefit” (OB; >30% decrease in BM blasts). Non-responders were individuals with either treatment failure (TF), stable disease (SD) or progressive disease (PD). Chemotherapy refractoriness was defined as ≥2 induction attempts or 1st CR with initial CR duration <6 months. Each IO 360 signature score was rescaled within the score for this cohort to a −3 to +3 scale to facilitate comparison across signatures.

BM samples from 92% of patients with evidence of anti-leukemic response (11 out of 12) to CD123×CD3 bispecific binding molecule therapy with flotetuzumab, had an immune-infiltrated TME relative to non-responders (FIG. 9).

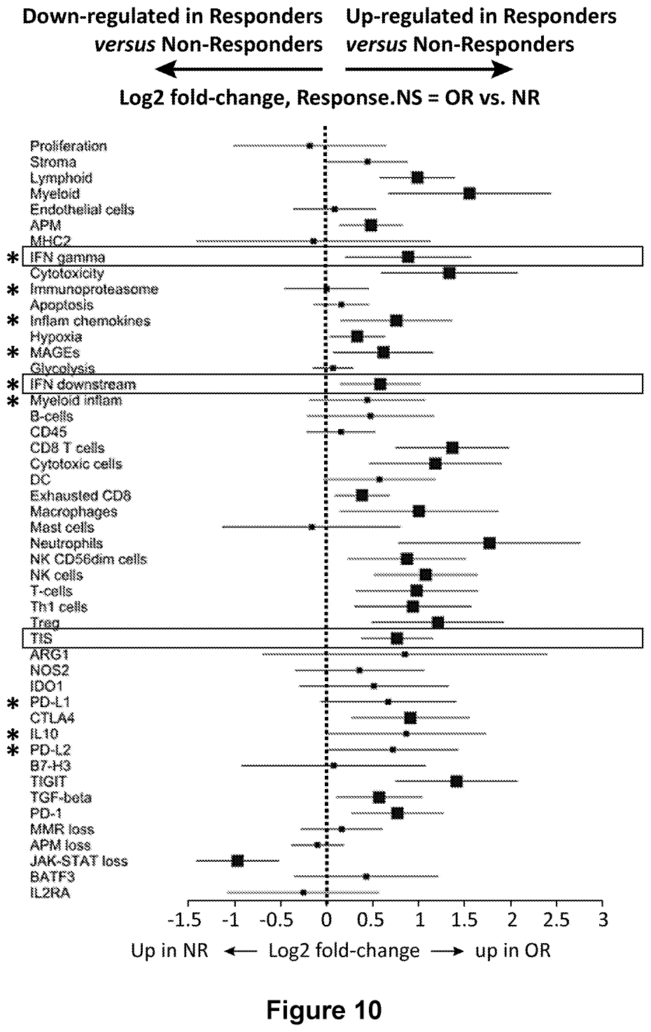

FIG. 10 presents a forest plot of the baseline fold change differences between responders (CR, CRi, CRh, PR, and OB) and non-responders (PD, SD, TF) from the analysis of the 30 chemotherapy-refractory or relapsed AML patients. Consistent with the analysis provided in Example 1 above, the expression of the IFN Gamma Signaling Signature, IFN Downstream Signature, and Tumor Inflammation Signature (boxed in FIG. 10) were increased in baseline samples in responders vs non-responders. In addition, most of the gene signatures that make up the IFN Dominant Module were increased in baseline samples in responders vs non-responders (starred in FIG. 10).

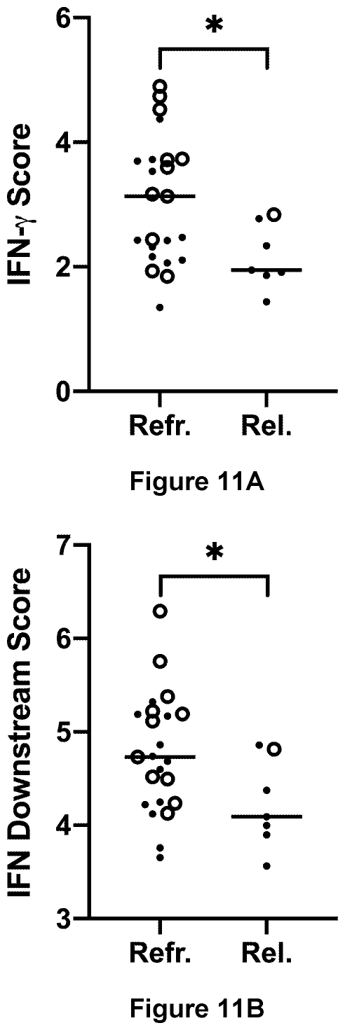

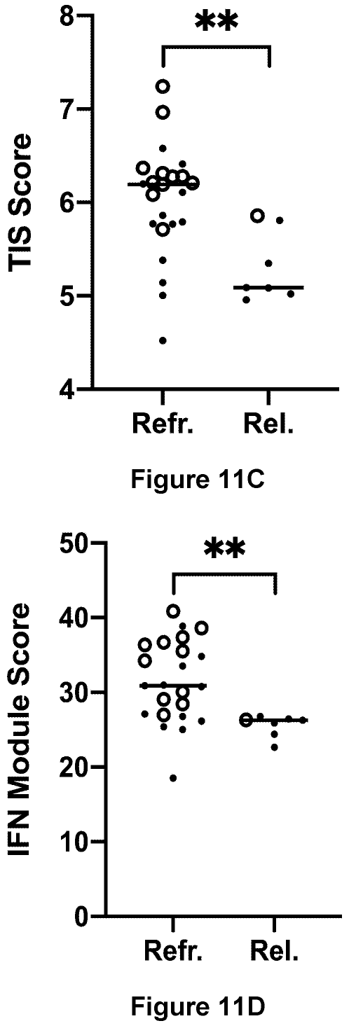

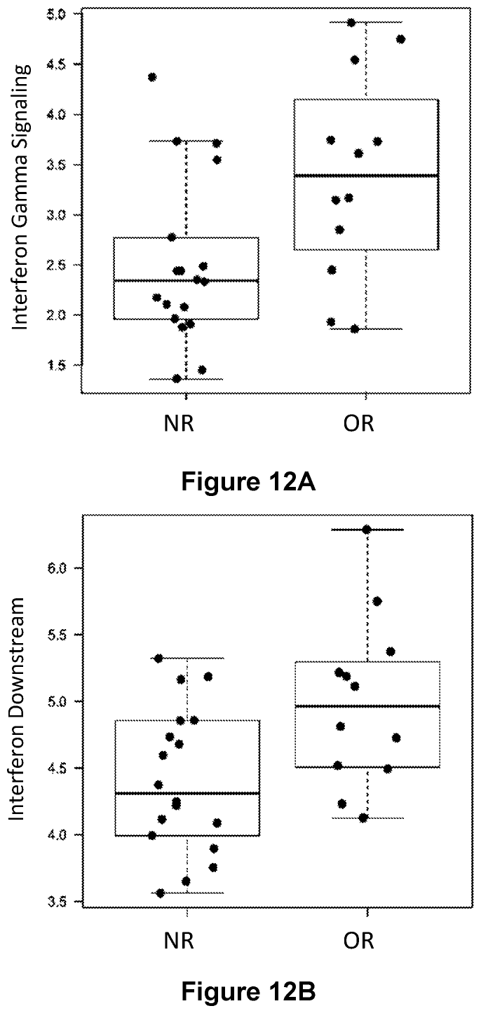

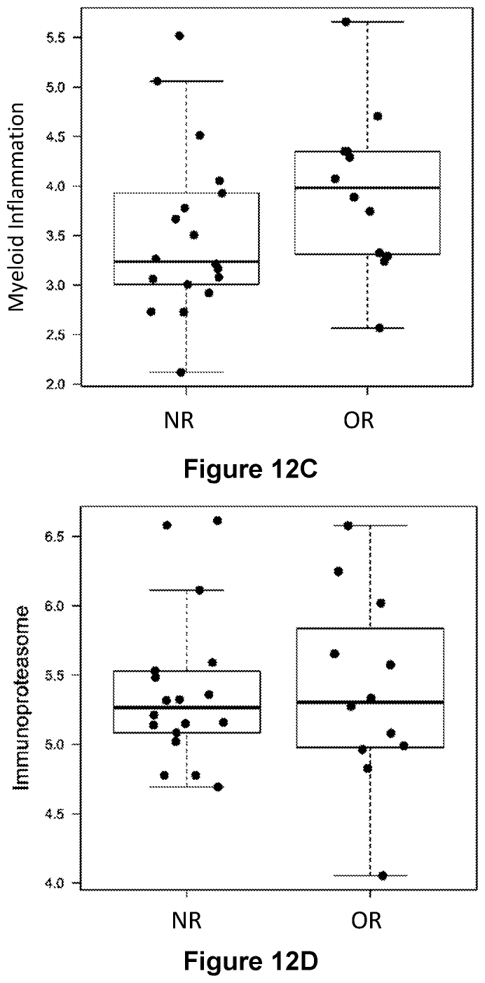

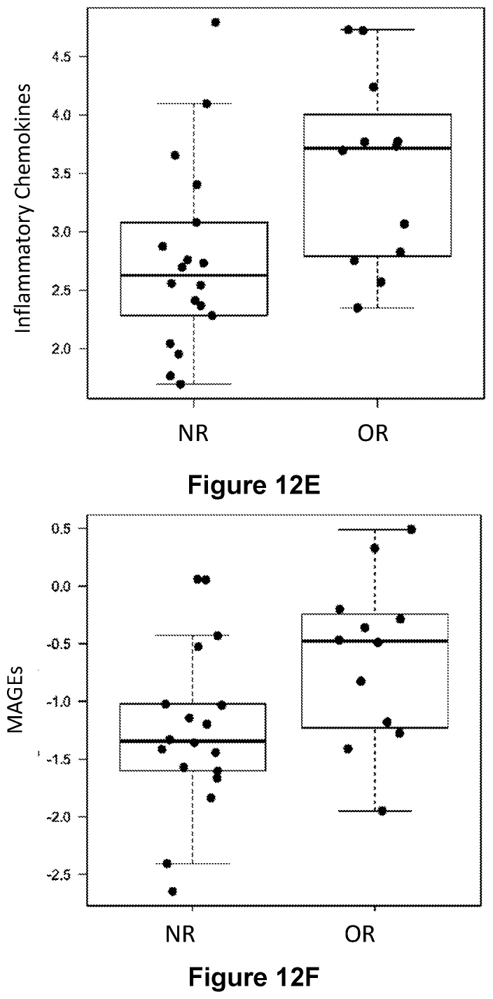

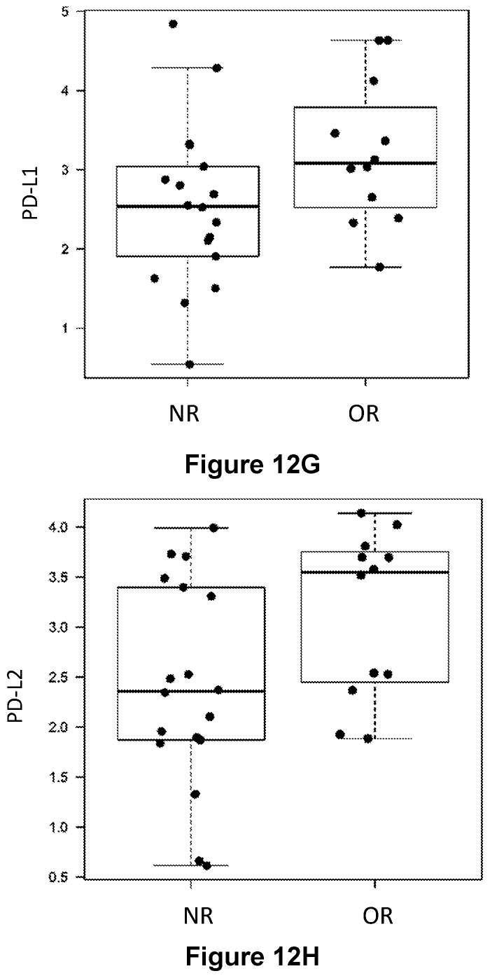

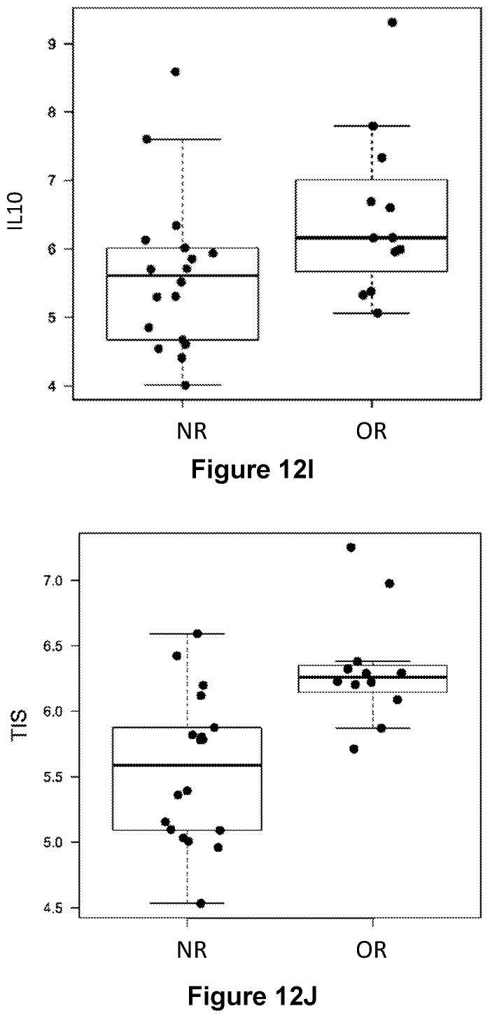

The distribution of the IFN Gamma Signaling Signature (FIG. 11A), the IFN Downstream Signature (FIG. 11B), the Tumor Inflammation Signature (TIS, FIG. 11C), and the IFN Dominant Module (FIG. 11D) scores between refractory versus relapsed patients are plotted in FIGS. 11A-11D. The distribution of scores are increased in refractory patients. The distribution of the scores of the nine gene signatures that make up the IFN Dominant Module and the Tumor Inflammation Signature in non-responders (NR) and responders (OR) are plotted in FIGS. 12A-12J: the IFN Gamma Signaling Signature (FIG. 12A); the IFN Downstream Signature (FIG. 12B); the Myeloid Inflammation Signature (FIG. 12C); the Immunoproteasome Signature (FIG. 12D); the Inflammatory Chemokines Signature (FIG. 12E); the MAGEs Signature (FIG. 12F); the PD-L1 Signature (FIG. 12G); the PD-L2 Signature (FIG. 12H); the IL10 Signature (FIG. 12I); the Tumor Inflammation Signature (TIS, FIG. 12J). The distribution of scores for these gene signatures are increased in responding patients. In particular, as shown in Table 11, the responders showed significantly higher IFN Gamma Signaling Signature, IFN Downstream Signature, TIS, and IFN Dominant Module scores at baseline compared to non-responders. Comparisons were performed with the Mann Whitney U test for unpaired determinations.

TABLE 11
Signature Scores (mean ± SD, Mann Whitney U test)

| | | Non-responder | |
|---|---|---|---|
| Signature | Responder Score | Score | p value |
| IFN Gamma Signaling | 3.38 ± 1.02 | 2.49 ± 0.82 | 0.0218 |
| IFN Downstream | 4.99 ± 0.63 | 4.41 ± 0.54 | 0.0193 |
| TIS | 6.31 ± 0.42 | 5.55 ± 0.57 | 0.0010 |
| IFN Dominant Module | 33.37 ± 4.95 | 27.84 ± 4.74 | 0.0043 |

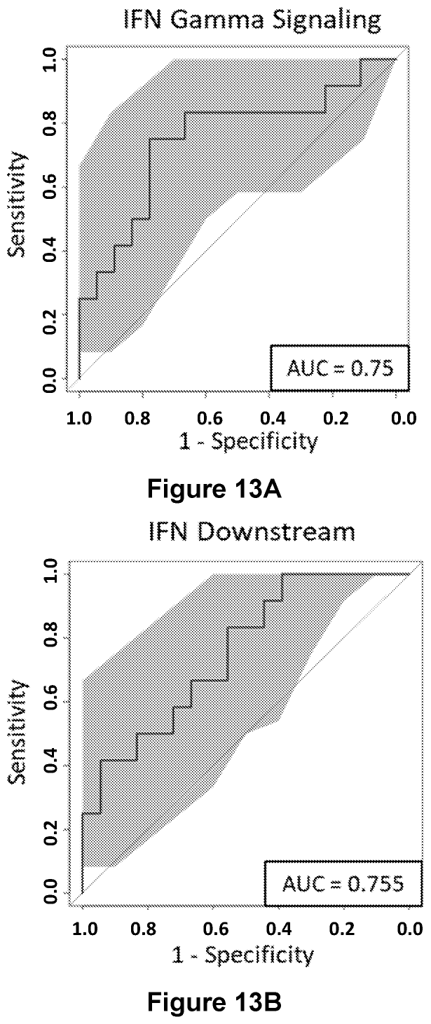

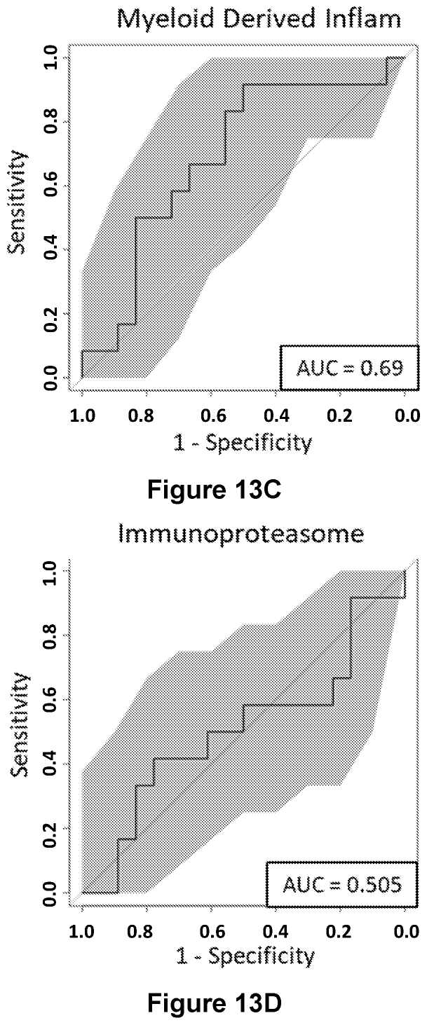

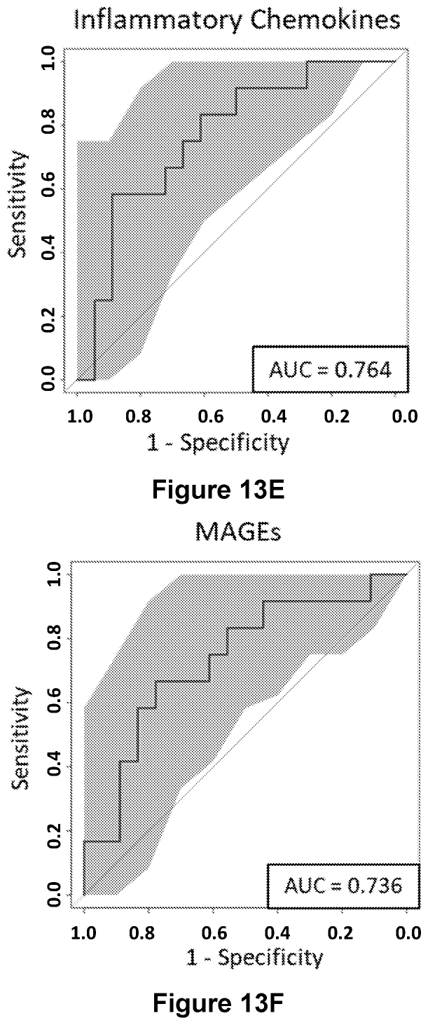

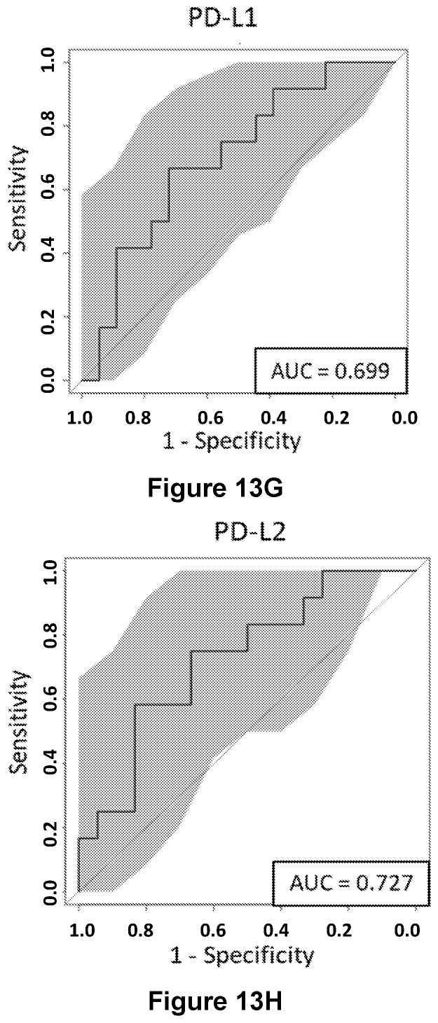

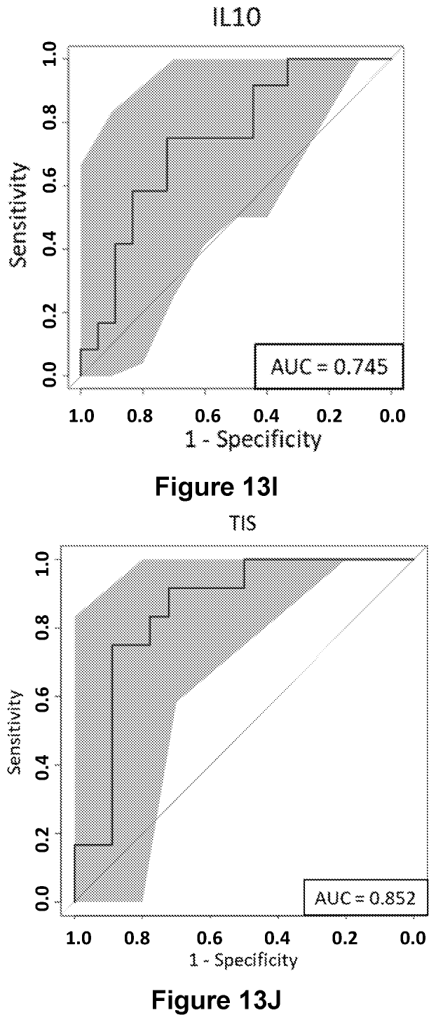

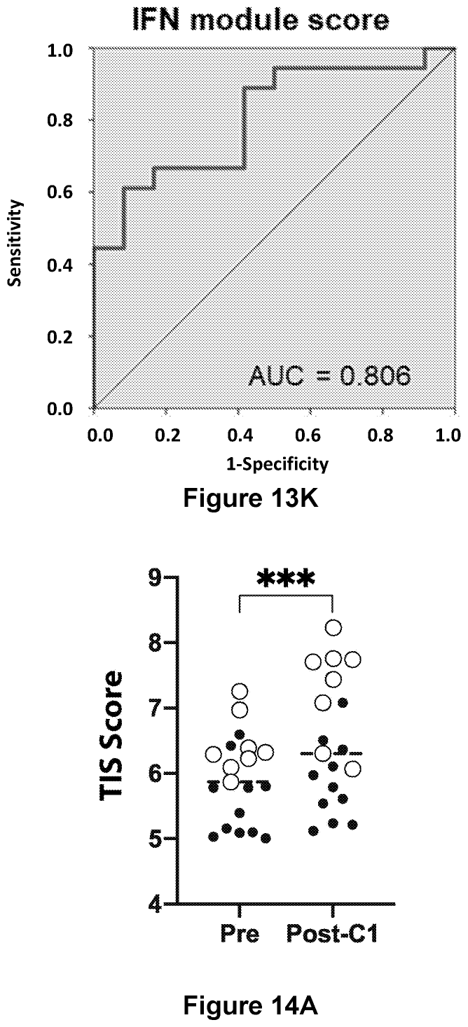

The sensitivity (true positive rate) and specificity (false positive rate) of the scores for the nine gene signatures that make up the IFN Dominant Module, the TIS, and the IFN Dominant Module for this group of patients were measured to predict response diagnostic capability (ROC AUC) essentially as described above. The ROC curves showing the predictive performance are presented in FIGS. 13A-13K: the IFN Gamma Signaling Signature (FIG. 13A, AUC=0.750); the IFN Downstream Signature (FIG. 13B, AUC=0.755); the Myeloid Inflammation Signature (FIG. 13C, AUC=0.69); the Immunoproteasome Signature (FIG. 13D, AUC=0.505); the Inflammatory Chemokines Signature (FIG. 13E, AUC=0.764); the MAGEs Signature (FIG. 13F, AUC=0.736); the PD-L1 Signature (FIG. 13G, AUC=0.699); the PD-L2 Signature (FIG. 13H, AUC=0.727); the IL10 Signature (FIG. 13I, AUC=0.745); the TIS (FIG. 13J, AUC=0.852), and IFN Dominant Module (FIG. 13K, AUC=0.806).

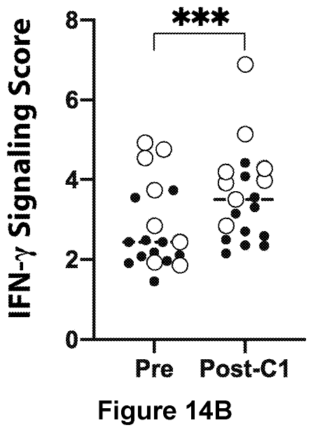

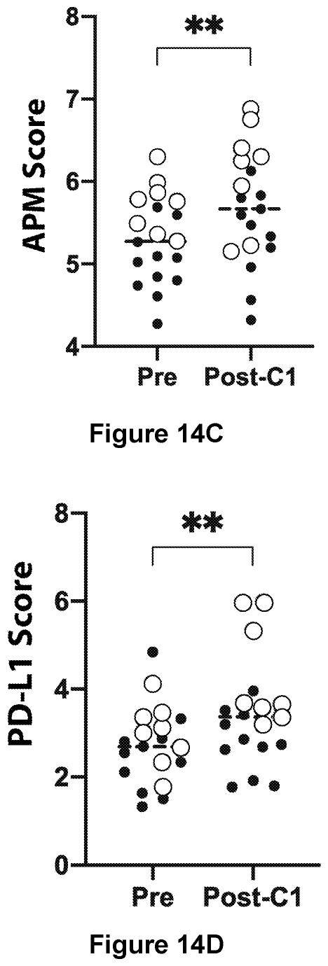

On-treatment BM samples (available in 19 patients at the end of cycle 1) displayed increased antigen presentation and immune activation relative to baseline samples (comparisons were performed with the Mann Whitney U test for unpaired determinations), as reflected by higher TIS scores (6.47±0.22 versus 5.93±0.15, p=0.0006, FIG. 14A), Antigen Processing Machinery (APM) Signature scores (5.67±0.16 versus 5.31±0.12, p=0.002, FIG. 14C), IFN-Gamma Signaling Signature scores (3.58±0.27 versus 2.81±0.24, p=0.0004, FIG. 14B) and PD-L1 Signature score (3.43±0.28 versus 2.73±0.21, p=0.0062; FIG. 14D). The results substantiate a clinical benefit for AML patients with an immune-infiltrated TME and support a local immune-modulatory effect of CD123×CD3 bispecific binding molecule therapy.

As noted above, it has been reported that AML patients with an immune-enriched and IFN gamma-dominant tumor microenvironment (“TME”) experience significantly shorter relapse-free survival, suggesting refractoriness to standard induction chemotherapy (Vadakekolathu, J. et al. (2017) “T Immune Gene Expression Profiling in Children and Adults with Acute Myeloid Leukemia Identifies Distinct Phenotypic Patterns,” Blood 130:3942A). These data indicate that that the IFN Gamma Signaling Signature, IFN Downstream Signature, and the IFN Dominant Module scores at baseline strongly correlate with refractoriness to standard chemotherapy and with response to CD123×CD3 bispecific binding molecule therapy. In addition, within the highly pre-treated individuals evaluated here (an average of 4 prior lines of therapy), most of gene signatures that make up the IFN Dominant Module and the Tumor Inflammation Signature (TIS) were shown to correlate with response to CD123×CD3 bispecific binding molecule therapy. Each of these scores were significantly higher in patients with chemotherapy-refractory AML compared with relapsed AML at time of treatment and in individuals with evidence of anti-leukemic activity compared to non-responders. The strong correlation is reflected by the ROC curves and AUC values.

### Gene Signatures

IO 360 gene counts were generated using the nCounter® system (NanoString Technologies, Inc.) essentially as follows: RNA (˜100 ng per sample) was purified from bone marrow aspirates, and was incubated with report and capture probe mix for hybridization. Transcript counts were analyzed on the nCounter FLEX analysis system using the high-resolution setting. Reporter code count (RCC) output files are used to calculate gene signature scores using pre-defined linear combinations (weighted averages) of biologically relevant gene sets essentially as previously described, as detailed herein.

The IFN Gamma Signaling Signature is described in detail above. Immune cell type abundance signatures were defined in Danaher, P., et al., 2017, “Gene Expression Markers of Tumor Infiltrating Leukocytes,” J Immunother Cancer 5, 18); Tumor Inflammation Signature is as described in Danaher, P., et al., 2018 (“Pan-cancer Adaptive Immune Resistance as Defined by the Tumor Inflammation Signature (TIS): Results From The Cancer Genome Atlas (TCGA),” J Immunother Cancer. 6(1):63) (also see T cell-inflamed GEP described in Ayers. M., et al. 2017, “IFN-γ—Related mRNA Profile Predicts Clinical Response to PD-1 blockade” J Clin Invest. 127(8):2930-2940, and WO 2016/094377), the other signatures are defined in Danaher, P., et al., (2018, “Development of Gene Expression Signatures Characterizing The Tumor-Immune Interaction,” J Clin Oncol 36, 205-205). For ease of reference the genes and weigh factors for selected Gene Signatures used in these studies are provided below.

The Tumor Inflammation Signature (TIS) genes (including a representative, non-limiting NCBI accession number for each gene), and weight factors (see, e.g. WO 2016/094377) are shown in Table 12A below.

TABLE 12A
The Tumor Inflammation Signature Genes

| Signature | Gene | NCBI Accession No. | Weight |
|---|---|---|---|
| TIS | CCL5 | NM_002985.2 | 0.008346 |
| TIS | CD27 | NM_001242.4 | 0.072293 |
| TIS | CD274 | NM_014143.3 | 0.042853 |
| TIS | CD276 | NM_001024736.1 | −0.0239 |
| TIS | CD8A | NM_001768.5 | 0.031021 |
| TIS | CMKLR1 | NM_004072.1 | 0.151253 |
| TIS | CXCL9 | NM_002416.1 | 0.074135 |
| TIS | CXCR6 | NM_006564.1 | 0.004313 |
| TIS | HLA-DQA1 | NM_002122.3 | 0.020091 |
| TIS | HLA-DRB1 | NM_002124.2 | 0.058806 |
| TIS | HLA-E | NM_005516.6 | 0.07175 |
| TIS | IDO1 | NM_002164.3 | 0.060679 |
| TIS | LAG3 | NM_002286.5 | 0.123895 |
| TIS | NKG7 | NM_005601.4 | 0.075524 |
| TIS | PDCD1LG2 | NM_025239.3 | 0.003734 |
| TIS | PSMB10 | NM_002801.2 | 0.032999 |
| TIS | STAT1 | NM_007315.2 | 0.250229 |
| TIS | TIGIT | NM_173799.2 | 0.084767 |

The Interferon (IFN) Downstream Signature Genes (including a representative, non-limiting NCBI accession number for each gene), and weight factors are shown in Table 12B below. The adjustment factor for this signature is: 5.342598.

TABLE 12B
The IFN Downstream Signaling Signature Genes

| Signature | Gene | NCBI Accession No. | Weight |
|---|---|---|---|
| IFN Downstream | APOL6 | NM_030641.4 | 0.03201 |
| IFN Downstream | DTX3L | NM_138287.3 | 0.04691 |
| IFN Downstream | GBP1 | NM_002053.1 | 0.0289 |
| IFN Downstream | IFI16 | NM_005531.1 | 0.02585 |
| IFN Downstream | IFI27 | NM_005532.5 | 0.02647 |
| IFN Downstream | IFI35 | NM_005533.3 | 0.05262 |
| IFN Downstream | IFI6 | NM_002038.4 | 0.03267 |
| IFN Downstream | IFIH1 | NM_022168.2 | 0.04021 |
| IFN Downstream | IFIT1 | NM_001548.5 | 0.03788 |
| IFN Downstream | IFIT2 | NM_001547.4 | 0.03232 |
| IFN Downstream | IFIT3 | NM_001549.6 | 0.0649 |
| IFN Downstream | IFITM1 | NM_003641.3 | 0.03325 |
| IFN Downstream | IFITM2 | NM_006435.2 | 0.02516 |
| IFN Downstream | IRF1 | NM_002198.1 | 0.03867 |
| IFN Downstream | IRF9 | NM_006084.5 | 0.06769 |
| IFN Downstream | ISG15 | NM_005101.4 | 0.03628 |
| IFN Downstream | MX1 | NM_002462.2 | 0.04467 |
| IFN Downstream | OAS1 | NM_016816.4 | 0.04457 |
| IFN Downstream | OAS2 | NM_002535.3 | 0.05578 |
| IFN Downstream | PARP9 | NM_001146104.2 | 0.05361 |
| IFN Downstream | PSMB9 | NM_002800.5 | 0.03815 |
| IFN Downstream | STAT2 | NM_005419.2 | 0.05018 |
| IFN Downstream | TMEM140 | NM_018295.5 | 0.03651 |
| IFN Downstream | TRIM21 | NM_003141.4 | 0.05474 |

The Inflammatory Chemokine (Inflam chemokines) Signature genes (including a representative, non-limiting NCBI accession number for each gene), and weight factors are shown in Table 12C below. The adjustment factor for this signature is: 6.0968.

TABLE 12C
The Inflam Chemokines Signature Genes

| Signature | Gene | NCBI Accession No. | Weight |
|---|---|---|---|
| Inflam chemokines | CCL2 | NM_002982.4 | 0.19758 |
| Inflam chemokines | CCL3/L1 | NM_021006.5 | 0.2053 |
| Inflam chemokines | CCL4 | NM_002984.2 | 0.23028 |
| Inflam chemokines | CCL7 | NM_006273.2 | 0.15535 |
| Inflam chemokines | CCL8 | NM_005623.2 | 0.21149 |

The MAGEs Signature genes (including a representative, non-limiting NCBI accession number for each gene), and weight factors are shown in Table 12D below. The adjustment factor for this signature is: 3.965625.

TABLE 12D
The MAGEs Signature Genes

| Signature | Gene | NCBI Accession No. | Weight |
|---|---|---|---|
| MAGEs | MAGEA3/A6 | NM_005362.4 | 0.30294 |
| MAGEs | MAGEA1 | NM_004988.5 | 0.11248 |
| MAGEs | MAGEA12 | NM_001166387.4 | 0.13496 |
| MAGEs | MAGEA4 | NM_001011549.1 | 0.0776 |
| MAGEs | MAGEB2 | NM_002364.5 | 0.11849 |
| MAGEs | MAGEC1 | NM_005462.5 | 0.12123 |
| MAGEs | MAGEC2 | NM_016249.4 | 0.12907 |

The Myeloid Inflammation (Myeloid Inflam) Signature genes (including a representative, non-limiting NCBI accession number for each gene), and weight factors are shown in Table 12E below. The adjustment factor for this signature is: 5.41931.

TABLE 12E
The Myeloid Inflam Signature Genes

| Signature | Gene | NCBI Accession No. | Weight |
|---|---|---|---|
| Myeloid inflam | AREG | NM_001657.4 | 0.06421 |
| Myeloid inflam | CSF3 | NM_172219.3 | 0.09023 |
| Myeloid inflam | CXCL1 | NM_001511.1 | 0.09222 |
| Myeloid inflam | CXCL2 | NM_002089.4 | 0.15153 |
| Myeloid inflam | CXCL3 | NM_002090.3 | 0.15227 |
| Myeloid inflam | CCL20 | NM_004591.3 | 0.06003 |
| Myeloid inflam | FOSL1 | NM_005438.5 | 0.0893 |
| Myeloid inflam | IER3 | NM_003897.4 | 0.13202 |
| Myeloid inflam | IL6 | NM_000600.5 | 0.09792 |
| Myeloid inflam | PTGS2 | NM_000963.4 | 0.07027 |

The Immunoproteasome Signature genes (including a representative, non-limiting NCBI accession number for each gene), and weight factors are shown in Table 12F below. The adjustment factor for this signature is: 6.096812.

TABLE 12F
The Immunoproteasome Signature Genes

| Signature | Gene | NCBI Accession No. | Weight |
|---|---|---|---|
| Immunoproteasome | PSMB8 | NM_004159.4 | 0.39749 |
| Immunoproteasome | PSMB9 | NM_002800.4 | 0.31826 |
| Immunoproteasome | PSMB10 | NM_002801.2 | 0.28426 |

The single gene signature genes (including a representative, non-limiting NCBI accession number for each gene) and adjustment factors are shown in Table 12G below.

TABLE 12G
Single Gene Signatures

| Signature | Gene | NCBI Accession No. | Adjustment Factor |
|---|---|---|---|
| IL10 | IL10 | NM_000572.3 | 9.6097 |
| PDL1 | CD274 | NM_014143.3 | 8.0352 |
| PDL2 | PDCD1LG2 | NM_025239.3 | 8.2984 |
| CTLA4 | CTLA4 | NM_005214.5 | 8.4925 |
| PD1 | PDCD1 | NM_005018.3 | 10.2306 |

The signatures scores are calculated essentially as described above except that once normalized and log transformed, each gene is multiplied to the weight provided in Tables 12A-12F, and the indicated adjustment factor is added. For single gene signatures (e.g., PDL1) no weight is used, the log 2 normalized gene expression values are added to the adjustment factors are provided in Table 12G.

All publications and patents mentioned in this specification are herein incorporated by reference to the same extent as if each individual publication or patent application was specifically and individually indicated to be incorporated by reference in its entirety. While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth.

What is claimed is:

1. A method of treating a chemo-refractory hematologic malignancy in a patient, wherein said method comprises administering to said patient a treatment dosage of a CD123×CD3 bispecific molecule, said dosage being effective to stimulate the killing of cells of said hematologic malignancy in said patient and thereby treat said malignancy.

2. The method of claim 1, wherein said method additionally comprises evaluating the expression of one or more target and/or reference genes in a cellular sample from said patient, prior to and/or subsequent to said administration of said CD123×CD3 bispecific molecule.

3. A method of determining whether a patient would be a suitable responder to the use of a CD123×CD3 bispecific molecule to treat a hematologic malignancy, wherein said method comprises:
(a) evaluating the expression of one or more target genes in a cellular sample from said patient prior to the administration of said CD123×CD3 bispecific molecule, relative to the expression of one or more target and/or reference genes; and
(b) identifying the patient as a suitable responder for treatment with a CD123×CD3 bispecific molecule if the expression of said one or more target genes is found to be increased relative to said expression of said one or more target and/or reference genes.

4. The method of any one of claims 2-3, wherein said method evaluates:
(i) the expression of one or more target genes; and
(ii) one or more reference genes whose expression is not characteristically associated with said hematologic malignancy.

5. The method of any one of claims 2-3, wherein said method comprises evaluating the expression of said one or more target genes relative to the baseline expression of said one or more reference genes of said patient.

6. The method of any one of claims 2-5, wherein said method comprises evaluating the expression of said one or more target genes of said patient relative to the expression of said one or more target genes of:
(a) an individual who is suffering from said hematologic malignancy, or of a population of such individuals; or
(b) an individual who did not successfully respond to the use of a CD123×CD3 bispecific molecule to treat said hematologic malignancy, or of a population of such individuals; or
(c) an individual who successfully responded to the use of a CD123×CD3 bispecific molecule to treat said hematologic malignancy, or of a population of such individuals.

7. The method of any one of claims 5-6, wherein the relative expression level of said one or more target genes in said population is established by averaging the gene expression level in cellular samples obtained from said population of individuals.

8. The method of any one of claims 2-7, wherein said patient exhibits an expression level of at least one of said target genes:
(a) that is greater than the first quartile of the expression levels of said target gene in a population of individuals who are suffering from said hematologic malignancy; or
(b) that is greater than the first quartile of the expression levels of said target gene in a population of individuals who did not successfully respond to a treatment for said hematologic malignancy that used a CD123×CD3 bispecific molecule; or
(c) that has a log₂-fold change of at least about 0.4 relative to the expression levels of said target gene in a population of individuals who did not successfully respond to a treatment for said hematologic malignancy that used a CD123×CD3 bispecific molecule; or
(d) that is within at least the first quartile of the expression levels of said target gene in a population of individuals who successfully responded to a treatment for said hematologic malignancy that used a CD123×CD3 bispecific molecule.

9. A method of treating a hematologic malignancy, wherein said method comprises:
(a) employing the method of any one of claims 3-8 to determine whether a patient would be a suitable responder to the use of a CD123×CD3 bispecific molecule to treat said hematologic malignancy;
(b) administering a treatment dosage of said CD123×CD3 bispecific molecule to said patient if said patient is determined to be a suitable responder to such treatment;
wherein said administration of said CD123×CD3 bispecific molecule stimulates the killing of cells of said hematologic malignancy in said patient.

10. The method of any one of claims 2-9, wherein said cellular sample is a bone marrow sample.

11. The method of any one of claims 2-10, wherein said evaluation of expression or said determination of whether said patient would be a suitable responder to the use of a CD123×CD3 bispecific molecule to treat a hematologic malignancy is performed by:
(a) determining the gene expression levels for each target gene in one or more cellular sample(s) using a gene expression platform; and
(b) comparing said target gene expression levels to the expression levels of one or more reference genes.

12. The method of any one of claims 2-11, wherein said evaluation of expression or said determination of whether said patient would be a suitable responder to the use of a CD123×CD3 bispecific molecule to treat a hematologic malignancy is performed by:
(a) measuring the raw RNA levels in for each target gene in one more cellular sample using a gene expression platform, wherein the gene expression platform comprises a reference gene set of housekeeping genes, and
(b) assigning a relative expression value, for each of the measured raw RNA levels for the target genes using the measured RNA levels of the internal reference genes.

13. The method of any one of claims 2-12, wherein said one or more target genes comprise:
(a) one or more of: CXCL9, CXCL10, CXCL11, and STAT1; and/or
(b) one or more of: CCL5, CD27, CD274, CD276, CD8A, CMKLR1, CXCL9, CXCR6, HLA-DQA1, HLA-DRB1, HLA-E, IDO1, LAG3, NKG7, PDCD1LG2, PSMB10, STAT1, and TIGIT; and/or
(c) one or more of: AREG, CSF3, CXCL1, CXCL2, CXCL3, CCL20, FOSL1, IER3 (NM_003897.4), IL6 and PTGS2; and/or
(d) one or more of: CCL2, CCL3/L1, CCL4, CCL7 and CCL8; and/or
(e) one or more of: MAGEA3/A6, MAGEA1, MAGEA12, MAGEA4, MAGEB2, MAGEC1 and MAGEC2; and/or
(f) one or more of: APOL6, DTX3L, GBP1, IE116, IE127, IE135, IFI6, IFIH1, IFIT1, IFIT2, IFIT3, IFITM1, IFITM2, IRF1, IRF9, ISG15, MX1, OAS1, OAS2, PARP9, PSMB9, STAT2, TMEM140 and TRIM21; and/or
(g) one or more of: PSMB8, PSMB9 and PSMB10; and/or
(h) IL-10; and or
(i) CD274; and/or
(j) PDCD1LG2.

14. The method of any one of claims 2-13, wherein said one or more reference genes comprise one or more of: ABCF1, G6PD, NRDE2, OAZ1, POLR2A, SDHA, STK11IP, TBC1D10B, TBP, and UBB.

15. The method of any one of claims 2-14, wherein a gene signature score is determined for said one or more target genes.

16. The method of claim 15, wherein said gene signature score is determined by a process comprising:
(a) measuring the raw RNA levels for each target gene in one more cellular sample using a gene expression platform comprising a reference gene set of housekeeping genes,
(b) normalizing each of the measured raw RNA levels to the geometric mean of said housekeeping genes, and optionally further normalizing each RNA value to a standard,
(c) log transforming each normalized RNA value,
(d) multiplying each log transformed RNA value by a corresponding weight factor to generate a weighted RNA value, and
(e) adding the weighted RNA values, and optionally adding an adjustment factor constant, to generate a single gene signature score.

17. The method of claim 15 or 16, wherein said gene signature score is determined using the target gene(s), the scoring weights and optionally the adjustment factors provided in Tables 6 and 12A-12G.

18. The method of any one of claims 15-17, wherein said gene signature score is a gene signature score determined for one or more of:
(a) the IFN Gamma Signaling Signature;
(b) the Tumor Inflammation Signature;
(c) the Myeloid Inflammation Signature;
(d) the Inflammatory Chemokine Signature;
(e) the MAGEs Signature;
(f) the IFN Downstream Signaling Signature;
(g) the Immunoproteasome Signature;
(h) the IL-10 Signature;
(i) the PD-L1 Signature; and/or
(j) the PD-L2 Signature.

19. The method of any one of claims 15-18, wherein a patient gene signature score that:
(a) is greater than the first quartile of scores for said gene signature calculated from the expression levels of one or more of said target genes in a population of individuals who are suffering from said hematologic malignancy; or
(b) is greater than the first quartile of scores for said gene signature calculated from the expression levels of one or more of said target genes in a population of individuals who did not successfully respond to a treatment for said hematologic malignancy that used a CD123×CD3 bispecific molecule; or
(c) has a log₂-fold change of at least about 0.4 relative to scores for said gene signature calculated from the expression levels of one or more of said target genes in a population of individuals who did not successfully respond to a treatment for said hematologic malignancy that used a CD123×CD3 bispecific molecule; or
(d) is within at least the first quartile of the scores for said gene signature calculated from the expression levels of one or more of said target genes in a population of individuals who successfully responded to a treatment for said hematologic malignancy that used a CD123×CD3 bispecific molecule,
is indicative of a more favorable patient response to treatment with said CD123×CD3 bispecific molecule.

20. The method of any one of claims 17-18, wherein:
(a) said gene signature is the IFN Gamma Signaling Signature, and a patient gene signature score of at least about 2.5 is indicative of a more favorable patient response to treatment with said CD123×CD3 bispecific molecule, and/or
(b) said gene signature is the Tumor Inflammation Signature, and a patient gene signature score of at least about 5.5 is indicative of a more favorable patient response to treatment with said CD123×CD3 bispecific molecule; and/or
(c) said gene signature is the IFN Downstream Signaling Signature, and a patient gene signature score of at least about 4.5 is indicative of a more favorable patient response to treatment with said CD123×CD3 bispecific molecule.

21. The method of any one of claims 17-19, wherein said gene signature is the IFN Gamma Signaling Signature, the Tumor Inflammation Signature, or the IFN Downstream Signaling Signature.

22. The method of any one of claims 17-21, wherein a patient that exhibits a gene expression signature that is characteristic of an immune-enriched and IFN gamma-dominant tumor microenvironment is indicative of a more favorable patient response to treatment with said CD123×CD3 bispecific molecule.

23. The method of any one of claims 1-22, wherein said CD123×CD3 bispecific molecule is a bispecific antibody or bispecific molecule comprising an scFv.

24. The method of claim 23, wherein said CD123×CD3 bispecific molecule is JNJ-63709178, XmAb14045 or APVO436.

25. The method of any one of claims 1-22, wherein said CD123×CD3 bispecific molecule is a covalently bonded bispecific diabody comprising:
(a) a first polypeptide chain having the amino acid sequence of SEQ ID NO:21; and
(b) a second polypeptide chain having the amino acid sequence of SEQ ID NO:23; and
wherein the first and said second polypeptide chains are covalently bonded to one another by a disulfide bond.

26. The method of any one of claims 1-25, wherein said hematologic malignancy of said patient is selected from the group consisting of: acute myeloid leukemia (AML), chronic myelogenous leukemia (CIVIL), blastic crisis of CML, Abelson oncogene-associated with CML (Bcr-ABL translocation), myelodysplastic syndrome (MDS), acute B lymphoblastic leukemia (B-ALL), acute T lymphoblastic leukemia (T-ALL), chronic lymphocytic leukemia (CLL), Richter's syndrome, Richter's transformation of CLL, hairy cell leukemia (HCL), blastic plasmacytoid dendritic cell neoplasm (BPDCN), non-Hodgkin's lymphoma (NHL), including mantle cell lymphoma (MCL) and small lymphocytic lymphoma (SLL), Hodgkin's lymphoma, systemic mastocytosis, and Burkitt's lymphoma.

27. The method of claim 26, wherein said hematologic malignancy of said patient is AML.

28. The method of any one of claims 2-27, wherein said hematologic malignancy of said patient is refractory to chemotherapy.

29. The method of any one of claim 1-4, or 6-38, wherein said treatment dosage of said CD123×CD3 bispecific molecule includes at least one dose selected from the group consisting of 30, 100, 300, and 500 ng/kg patient weight/day.

30. The method of any one of claim 1-2, or 4-29, wherein said treatment dosage is administered by continuous infusion.

31. The method of any one of claims 1-30, wherein said patient is a human patient.


SEQUENCE LISTING (30 sequences)

SEQ ID NO: 1 - protein, 110 aa, Artificial Sequence
  VLCD3 Light Chain Variable Domain of Humanized CD123 x CD3 Bispecific Antibody Flotuzumab (DART-A)
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLG

SEQ ID NO: 2 - protein, 14 aa, Artificial Sequence
  CDRL1 of VLCD3 Light Chain Variable Domain of Humanized CD123 x CD3 Bispecific Antibody Flotuzumab (DART-A)
RSSTGAVTTSNYAN

SEQ ID NO: 3 - protein, 7 aa, Artificial Sequence
  CDRL2 of VLCD3 Light Chain Variable Domain of Humanized CD123 x CD3 Bispecific Antibody Flotuzumab (DART-A)
GTNKRAP

SEQ ID NO: 4 - protein, 9 aa, Artificial Sequence
  CDRL3 of VLCD3 Light Chain Variable Domain of Humanized CD123 x CD3 Bispecific Antibody Flotuzumab (DART-A)
ALWYSNLWV

SEQ ID NO: 5 - protein, 8 aa, Artificial Sequence
  Linker 1
GGGSGGGG

SEQ ID NO: 6 - protein, 120 aa, Artificial Sequence
  VHCD123 Heavy Chain Variable Domain of Humanized CD123 x CD3 Bispecific Antibody Flotuzumab (DART-A)
EVQLVQSGAELKKPGASVKVSCKASGYTFTDYYMKWVRQAPGQGLEWIGDIIPSNGATFYNQKFKGRVTITVDKSTSTAYMELSSLRSEDTAVYYCARSHLLRASWFAYWGQGTLVTVSS

SEQ ID NO: 7 - protein, 5 aa, Artificial Sequence
  CDRH1 of VHCD123 Heavy Chain Variable Domain of Humanized CD123 x CD3 Bispecific Antibody Flotuzumab (DART-A)
DYYMK

SEQ ID NO: 8 - protein, 17 aa, Artificial Sequence
  CDRH2 of VHCD123 Heavy Chain Variable Domain of Humanized CD123 x CD3 Bispecific Antibody Flotuzumab (DART-A)
DIIPSNGATFYNQKFKG

SEQ ID NO: 9 - protein, 11 aa, Artificial Sequence
  CDRH3 of VHCD123 Heavy Chain Variable Domain of Humanized CD123 x CD3 Bispecific Antibody Flotuzumab (DART-A)
SHLLRASWFAY

SEQ ID NO: 10 - protein, 113 aa, Artificial Sequence
  VLCD123 Light Chain Variable Domain of Humanized CD123 x CD3 Bispecific Antibody Flotuzumab (DART-A)
DFVMTQSPDSLAVSLGERVTMSCKSSQSLLNSGNQKNYLTWYQQKPGQPPKLLIYWASTRESGVPDRFSGSGSGTDFTLTISSLQAEDVAVYYCQNDYSYPYTFGQGTKLEIK

SEQ ID NO: 11 - protein, 17 aa, Artificial Sequence
  CDRL1 of VLCD123 Light Chain Variable Domain of Humanized CD123 x CD3 Bispecific Antibody Flotuzumab (DART-A)
KSSQSLLNSGNQKNYLT

SEQ ID NO: 12 - protein, 7 aa, Artificial Sequence
  CDRL2 of VLCD123 Light Chain Variable Domain of Humanized CD123 x CD3 Bispecific Antibody Flotuzumab (DART-A)
WASTRES

SEQ ID NO: 13 - protein, 9 aa, Artificial Sequence
  CDRL3 of VLCD123 Light Chain Variable Domain of Humanized CD123 x CD3 Bispecific Antibody Flotuzumab (DART-A)
QNDYSYPYT

SEQ ID NO: 14 - protein, 125 aa, Artificial Sequence
  VHCD3 Heavy Chain Variable Domain of Humanized CD123 x CD3 Bispecific Antibody Flotuzumab (DART-A)
EVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCVRHGNFGNSYVSWFAYWGQGTLVTVSS

SEQ ID NO: 15 - protein, 5 aa, Artificial Sequence
  CDRH1 of VHCD3 Heavy Chain Variable Domain of Humanized CD123 x CD3 Bispecific Antibody Flotuzumab (DART-A)
TYAMN

SEQ ID NO: 16 - protein, 19 aa, Artificial Sequence
  CDRH2 of VHCD3 Heavy Chain Variable Domain of Humanized CD123 x CD3 Bispecific Antibody Flotuzumab (DART-A)
RIRSKYNNYATYYADSVKD

SEQ ID NO: 17 - protein, 14 aa, Artificial Sequence
  CDRH3 of VHCD3 Heavy Chain Variable Domain of Humanized CD123 x CD3 Bispecific Antibody Flotuzumab (DART-A)
HGNFGNSYVSWFAY

SEQ ID NO: 18 - protein, 6 aa, Artificial Sequence
  Linker 2
GGCGGG

SEQ ID NO: 19 - protein, 28 aa, Artificial Sequence
  “E-Coil” Heterodimer-Promoting Domain
EVAALEKEVAALEKEVAALEKEVAALEK

SEQ ID NO: 20 - protein, 28 aa, Artificial Sequence
  “K-Coil” Heterodimer-Promoting Domain
KVAALKEKVAALKEKVAALKEKVAALKE

SEQ ID NO: 21 - protein, 272 aa, Artificial Sequence
  First Polypeptide Chain of Humanized CD123 x CD3 Bispecific Antibody Flotuzumab (DART-A)
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGGGGSGGGGEVQLVQSGAELKKPGASVKVSCKASGYTFTDYYMKWVRQAPGQGLEWIGDIIPSNGATFYNQKFKGRVTITVDKSTSTAYMELSSLRSEDTAVYYCARSHLLRASWFAYWGQGTLVTVSSGGCGGGEVAALEKEVAALEKEVAALEKEVAALEK

SEQ ID NO: 22 - DNA, 816 bp, Artificial Sequence
  Polynucleotide Encoding First Polypeptide Chain of Humanized CD123 x CD3 Bispecific Antibody Flotuzumab (DART-A)
caggctgtggtgactcaggagccttcactgaccgtgtccccaggcggaactgtgaccctgacatgcagatccagcacaggcgcagtgaccacatctaactacgccaattgggtgcagcagaagccaggacaggcaccaaggggcctgatcgggggtacaaacaaaagggctccctggacccctgcacggttttctggaagtctgctgggcggaaaggccgctctgactattaccggggcacaggccgaggacgaagccgattactattgtgctctgtggtatagcaatctgtgggtgttcgggggtggcacaaaactgactgtgctgggagggggtggatccggcggcggaggcgaggtgcagctggtgcagtccggggctgagctgaagaaacccggagcttccgtgaaggtgtcttgcaaagccagtggctacaccttcacagactactatatgaagtgggtcaggcaggctccaggacagggactggaatggatcggcgatatcattccttccaacggggccactttctacaatcagaagtttaaaggcagggtgactattaccgtggacaaatcaacaagcactgcttatatggagctgagctccctgcgctctgaagatacagccgtgtactattgtgctcggtcacacctgctgagagccagctggtttgcttattggggacagggcaccctggtgacagtgtcttccggaggatgtggcggtggagaagtggccgcactggagaaagaggttgctgctttggagaaggaggtcgctgcacttgaaaaggaggtcgcagccctggagaaa

SEQ ID NO: 23 - protein, 280 aa, Artificial Sequence
  Second Polypeptide Chain of Humanized CD123 x CD3 Bispecific Antibody Flotuzumab (DART-A)
DFVMTQSPDSLAVSLGERVTMSCKSSQSLLNSGNQKNYLTWYQQKPGQPPKLLIYWASTRESGVPDRFSGSGSGTDFTLTISSLQAEDVAVYYCQNDYSYPYTFGQGTKLEIKGGGSGGGGEVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCVRHGNFGNSYVSWFAYWGQGTLVTVSSGGCGGGKVAALKEKVAALKEKVAALKEKVAALKE

SEQ ID NO: 24 - DNA, 840 bp, Artificial Sequence
  Polynucleotide Encoding Second Polypeptide Chain of Humanized CD123 x CD3 Bispecific Antibody Flotuzumab (DART-A)
gacttcgtgatgacacagtctcctgatagtctggccgtgagtctgggggagcgggtgactatgtcttgcaagagctcccagtcactgctgaacagcggaaatcagaaaaactatctgacctggtaccagcagaagccaggccagccccctaaactgctgatctattgggcttccaccagggaatctggcgtgcccgacagattcagcggcagcggcagcggcacagattttaccctgacaatttctagtctgcaggccgaggacgtggctgtgtactattgtcagaatgattacagctatccctacactttcggccaggggaccaagctggaaattaaaggaggcggatccggcggcggaggcgaggtgcagctggtggagtctgggggaggcttggtccagcctggagggtccctgagactctcctgtgcagcctctggattcaccttcagcacatacgctatgaattgggtccgccaggctccagggaaggggctggagtgggttggaaggatcaggtccaagtacaacaattatgcaacctactatgccgactctgtgaaggatagattcaccatctcaagagatgattcaaagaactcactgtatctgcaaatgaacagcctgaaaaccgaggacacggccgtgtattactgtgtgagacacggtaacttcggcaattcttacgtgtcttggtttgcttattggggacaggggacactggtgactgtgtcttccggaggatgtggcggtggaaaagtggccgcactgaaggagaaagttgctgctttgaaagagaaggtcgccgcacttaaggaaaaggtcgcagccctgaaagag

SEQ ID NO: 25 - protein, 217 aa, Artificial Sequence
  “Knob-Bearing” CH2 and CH3 Fc Domain
  MISC_FEATURE (217)..(217): XAA is Lys (K) or is Absent
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGX

SEQ ID NO: 26 - protein, 217 aa, Artificial Sequence
  “Hole-Bearing” CH2 and CH3 Fc Domain
  MISC_FEATURE (217)..(217): XAA is Lys (K) or is Absent
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGX

SEQ ID NO: 27 - protein, 510 aa, Artificial Sequence [sequence omitted]

SEQ ID NO: 28 - protein, 272 aa, Artificial Sequence
  Second Polypeptide Chain of DART-A w/Fc Version 1 Construct
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGGGGSGGGGEVQLVQSGAELKKPGASVKVSCKASGYTFTDYYMKWVRQAPGQGLEWIGDIIPSNGATFYNQKFKGRVTITVDKSTSTAYMELSSLRSEDTAVYYCARSHLLRASWFAYWGQGTLVTVSSGGCGGGKVAALKEKVAALKEKVAALKEKVAALKE

SEQ ID NO: 29 - protein, 10 aa, Artificial Sequence
  Peptide 1
DKTHTCPPCP

SEQ ID NO: 30 - protein, 227 aa, Artificial Sequence
  Polypeptide Containing Peptide 1 (SEQ ID NO29) and “Hole-Bearing” CH2 and CH3 Fc Domain (SEQ ID NO26)
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK